(19) United States
(12) Patent Application Publication
(10) Pub. No.: US 2012/0303080 A1
(43) Pub. Date: Nov. 29, 2012

(54) PARASYMPATHETIC NERVE STIMULATION

(75) Inventors: Tamir BEN-DAVID, Tel Aviv (IL); Omry BEN-EZRA, Tel Aviv (IL); Ehud COHEN, Ganei Tikva (IL)

(73) Assignee: BIO CONTROL MEDICAL (B.C.M.) LTD., Yehud (IL)

(21) Appl. No.: 13/493,499

(22) Filed: Jun. 11, 2012

Related U.S. Application Data
(63) Continuation-in-part of application No. 11/657,784, filed on Jan. 24, 2007, now Pat. No. 8,204,591.
Application No. 11/657,784 is a continuation-in-part of application No. 10/866,601, filed on Jun. 10, 2004, now abandoned.
Application No. 11/657,784 is a continuation-in-part of application No. 10/560,654, filed on May 1, 2006, now Pat. No. 7,885,711.
Application No. 10/560,654 is a continuation-in-part of application No. 10/461,696, filed on Jun. 13, 2003, now Pat. No. 7,321,793.
Application No. 11/657,784 is a continuation-in-part of application No. 11/359,266, filed on Feb. 21, 2006, now Pat. No. 8,036,745.
Application No. 11/359,266 is a continuation-in-part of application No. 10/866,601, filed on Jun. 10, 2004, now abandoned.
(60) Provisional application No. 60/478,576, filed on Jun. 13, 2003.
(60) Provisional application No. 60/655,604, filed on Feb. 22, 2005.

(51) Int. Cl. A61N 1/365; A61N 1/05

(52) U.S. Cl. 607/14; 607/119

(57) ABSTRACT

A method is provided, including identifying that a subject is at risk of suffering from atrial fibrillation (AF). Responsively to the identifying, a risk of an occurrence of an episode of the AF is reduced by coupling an electrode device to a site of a subject containing parasympathetic nervous tissue; driving, by a control unit, the electrode device to apply an electrical current to the site not responsively to any physiological parameters sensed by any device directly or indirectly coupled to the control unit; and configuring the current to stimulate autonomic nervous tissue in the site. Other embodiments are also described.

### CROSS-REFERENCES TO RELATED APPLICATIONS

This application is a continuation-in-part of U.S. patent application Ser. No. 11/657,784, filed Jan. 24, 2007, which published as US Patent Application Publication 2007/0179543, and which is a continuation-in-part of:

(a) U.S. patent application Ser. No. 10/866,601, filed Jun. 10, 2004, which published as US Patent Application Publication 2005/0065553, and which claims the benefit of claims of U.S. Provisional Patent Application 60/478,576, filed Jun. 13, 2003;

(b) U.S. patent application Ser. No. 10/560,654, filed May 1, 2006, now U.S. Pat. No. 7,885,711, which is the U.S. national stage of PCT Patent Application PCT/IL04/00496, filed Jun. 10, 2004, which published as PCT Publication WO 04/110550, which is a continuation-in-part of U.S. patent application Ser. No. 10/461,696, filed Jun. 13, 2003, now U.S. Pat. No. 7,321,793; and

(c) U.S. patent application Ser. No. 11/359,266, filed Feb. 21, 2006, now U.S. Pat. No. 8,036,745, which: (1) claims the benefit of U.S. Provisional Patent Application 60/655,604, filed Feb. 22, 2005, and (2) is a continuation-in-part of U.S. patent application Ser. No. 10/866,601, filed Jun. 10, 2004, which published as US Patent Application Publication 2005/0065553.

All of the above-mentioned applications are assigned to the assignee of the present application, and are incorporated herein by reference.

### FIELD OF THE INVENTION

The present invention relates generally to treating patients by application of electrical signals to selected tissue, and specifically to methods and apparatus for stimulating tissue for treating patients suffering from conditions such as atrial fibrillation.

### BACKGROUND OF THE INVENTION

The use of nerve stimulation for treating and controlling a variety of medical, psychiatric, and neurological disorders has seen significant growth over the last several decades, including for treatment of heart conditions. In particular, stimulation of the vagus nerve (the tenth cranial nerve, and part of the parasympathetic nervous system) has been the subject of considerable research. The vagus nerve is composed of somatic and visceral afferents (inward conducting nerve fibers, which convey impulses toward the brain) and efferents (outward conducting nerve fibers, which convey impulses to an effector to regulate activity such as muscle contraction or glandular secretion).

The rate of the heart is restrained in part by parasympathetic stimulation from the right and left vagus nerves. Low vagal nerve activity is considered to be related to various arrhythmias, including tachycardia, ventricular accelerated rhythm, and atrial fibrillation with rapid ventricular response. By artificially stimulating the vagus nerves, it is possible to slow the heart, allowing the heart to more completely relax and the ventricles to experience increased filling. With larger diastolic volumes, the heart may beat more efficiently because it may expend less energy to overcome the myocardial viscosity and elastic forces of the heart with each beat.

Stimulation of the vagus nerve has been proposed as a method for treating various heart conditions, including atrial fibrillation and heart failure. Atrial fibrillation is a condition in which the atria of the heart fail to continuously contract in synchrony with the ventricles of the heart. During fibrillation, the atria undergo rapid and unorganized electrical depolarization, so that no contractile force is produced. The ventricles, which normally receive contraction signals from the atria (through the atrioventricular (AV) node), are inundated with signals, typically resulting in a rapid and/or irregular ventricular rate. Because of this rapid and irregular rate, the patient suffers from reduced cardiac output and/or a feeling of palpitations.

Current therapy for atrial fibrillation includes cardioversion and rate control. Cardioversion is the conversion of the abnormal atrial rhythm into normal sinus rhythm. This conversion is generally achieved pharmacologically or electrically. Rate control therapy is used to control the ventricular rate, while allowing the atria to continue fibrillation. This is generally achieved by slowing the conduction of signals through the AV node from the atria to the ventricles.

After cardioversion has been successfully performed, drug therapy is sometimes indicated for sinus rhythm maintenance or ventricular rate control (see Fuster et al., in their articles cited hereinbelow). Commonly used antiarrhythmic drugs for prophylactic maintenance of sinus rhythm include beta-blockers, amiodarone, disopyramide, dofetilide, flecamide, procainamide, propafenone, quinidine, and sotalol. Potential adverse effects of these drugs include hypotension, bradycardia, QT prolongation, ventricular proarrhythmia (ventricular tachycardia, including torsades de pointes), postural hypotension, and GI complaints, such as diarrhea. For ventricular rate control, commonly used drugs include beta-blockers (e.g., esmolol), calcium channel antagonists (e.g., verapamil, diltiazem) and digoxin. Potential adverse effects of these drugs include hypotension, heart block, heart failure, and bradycardia.

Bilgutay et al., in “Vagal tuning: a new concept in the treatment of supraventricular arrhythmias, angina pectoris, and heart failure,” J. Thoracic Cardiovas. Surg. 56(1):71-82, July, 1968, studied the use of a permanently-implanted device with electrodes to stimulate the right vagus nerve for treatment of supraventricular arrhythmias, angina pectoris, and heart failure. Experiments were conducted to determine amplitudes, frequencies, wave shapes and pulse lengths of the stimulating current to achieve slowing of the heart rate. The authors additionally studied an external device, triggered by the R-wave of the electrocardiogram (ECG) of the subject to provide stimulation only upon an achievement of a certain heart rate. They found that when a pulsatile current with a frequency of ten pulses per second and 0.2 milliseconds pulse duration was applied to the vagus nerve, the heart rate could be decreased to half the resting rate while still preserving sinus rhythm. Low amplitude vagal stimulation was employed to control induced tachycardias and ectopic beats. The authors further studied the use of the implanted device in conjunction with the administration of Isuprel, a sympathomimetic drug. They found that Isuprel retained its inotropic effect of increasing contractility, while its chronotropic effect was controlled by the vagal stimulation: “An increased end diastolic volume brought about by slowing of the heart rate by vagal tuning, coupled with increased contractility of the heart induced by the inotropic effect of Isuprel, appeared to increase the efficiency of cardiac performance” (p. 79).

The effect of vagal stimulation on heart rate and other aspects of heart function, including the relationship between the timing of vagal stimulation within the cardiac cycle and the induced effect on heart rate, has been studied in animals. For example, Zhang Y et al., in “Optimal ventricular rate slowing during atrial fibrillation by feedback AV nodal-selective vagal stimulation,” Am J Physiol Heart Circ Physiol 282:H1102-H1110 (2002), describe the application of selective vagal stimulation by varying the nerve stimulation intensity, in order to achieve graded slowing of heart rate.

A number of patents describe techniques for treating arrhythmias and/or ischemia by, at least in part, stimulating the vagus nerve. Arrhythmias in which the heart rate is too fast include fibrillation, flutter and tachycardia. Arrhythmia in which the heart rate is too slow is known as bradyarrhythmia. U.S. Pat. No. 5,700,282 to Zabara describes techniques for stabilizing the heart rhythm of a patient by detecting arrhythmias and then electronically stimulating the vagus and cardiac sympathetic nerves of the patient. The stimulation of vagus efferents directly causes the heart rate to slow down, while the stimulation of cardiac sympathetic nerve efferents causes the heart rate to quicken.

### SUMMARY OF THE INVENTION

In some embodiments of the present invention, a method for treating a subject at risk of suffering from atrial fibrillation (AF) comprises reducing the risk of an occurrence of an episode of the AF by applying an electrical current to a vagus nerve or other parasympathetic tissue that innervates the heart of the subject. Apparatus is provided for applying the electrical current, comprising an electrode device and a control unit, which is configured to drive the electrode device to apply the current.

Typically, the control unit is configured to apply the current on a chronic, long-term basis, even when the subject is not currently experiencing an episode of the AF, and even in the absence of a prediction of an imminent episode of the AF. The current is thus typically applied during normal sinus rhythm (NSR). For some applications, the control unit applies the current not responsively to any physiological parameters sensed by the control unit or a sensor coupled to the control unit. For some applications, the control unit applies the current not responsively to any measure of heart rate of the subject (which may be expressed as a heart rate or interval, e.g., an R-R interval) determined by the control unit. For these application, the control unit does not configure any parameters of the applied current responsively to any measure of the heart rate, including any timing parameters of the current application.

The control unit typically does not configure the current to achieve regulation of a heart rate of the subject, such as to achieve a target heart rate or range. For some applications, the current is configured to minimize an effect of the applying of the current on a heart rate of the subject.

For some applications, the control unit configures the current to delay electrical remodeling of an atrium of the subject, to reduce mechanical stress of a heart of the subject, and/or to induce rhythmic vagal activity.

In some embodiments of the present invention, upon sensing an occurrence of an episode of the AF, the control unit reduces a strength of the current, e.g., withholds applying the current, typically during a strength reduction period having a duration of at least one minute, e.g., at least 5 minutes, at least 10 minutes, at least 20 minutes, or at least one hour. The inventors believe that application of the current sometimes prolongs episodes of AF, so reducing the strength of or withholding the current generally allows episodes to resolve more quickly than they would during application of the current at full strength. Similarly, for some applications, upon predicting an imminent episode of the AF, the control unit reduces the strength of the current, e.g., withholds applying the current. For some applications, upon conclusion of the strength reduction period, the control unit configures the current to reduce a heart rate of the subject if the episode of AF has not terminated, and the subject has an elevated heart rate.

In some embodiments of the present invention, the control unit applies the current in a series of bursts, each of which bursts includes at least one pulse. For some applications, the control unit synchronizes at least a portion of the bursts with a feature of a cardiac cycle of the subject, such as a P-wave or R-wave. Synchronization with the P-wave has the effect of automatically withholding stimulation during AF, because no P-wave is present during AF.

In some embodiments of the present invention, the subject is determined to be at risk of suffering from AF by identifying that the subject suffers from at least one of the following conditions:
paroxysmal AF;
self-terminating AF episodes;
an enlarged atrium;
multiple atrial premature beats (APBs);
mitral stenosis;
heart failure;
thyrotoxicosis;
hypertension; and
atrial flutter.

Alternatively or additionally, the subject is determined to be at risk of suffering from AF by identifying that the subject has undergone an interventional heart procedure, such as coronary bypass surgery or valve replacement surgery.

For some applications, this determination is made after the subject has suffered from at least one episode of the AF, while for other applications, the determination is made prior to the subject suffering from any known episodes of the AF.

In some embodiments of the present invention, a method for enhancing or sustaining the efficacy of drug treatment for atrial fibrillation (AF) comprises administering a drug to a patient and applying signals to a vagus nerve that innervates the heart of the patient. The drug administered typically includes a sinus rhythm maintenance drug (i.e., an antiarrhythmic drug) or a ventricular rate control drug. The efficacy of the drug is typically enhanced or sustained by (a) configuring the signals so as to prevent electrical remodeling of the atria, which remodeling generally reduces drug effectiveness over time, and/or (b) configuring the signals so as to achieve a therapeutic benefit similar to that of the drug, which typically results in a synergistic effect between the therapeutic benefit of the drug and the vagal stimulation. For enhancing the effectiveness of antiarrhythmic drugs, the signals are typically configured to increase vagal tone, produce rhythmic vagal activity, and/or reduce the atrial effective refractory period (AERP). The effectiveness of ventricular rate control drugs is typically enhanced by applying vagal stimulation to control ventricular response rate and/or to improve cardiac output.

In some embodiments of the present invention, a method for enhancing or sustaining the efficacy of drug treatment for AF comprises administering a drug to the patient, applying signals to the vagus nerve, and configuring the signals to reduce the mechanical tension on at least one atrium of the subject. Such reduced mechanical tension generally reduces the risk of AF. For some applications, such vagal stimulation is applied without administering the drug.

In some embodiments of the present invention, the safety of a drug administered to the patient is improved by applying signals to the vagus nerve, and configuring the signals so as to prevent adverse effects sometimes caused by the drug, such as repolarization abnormalities (e.g., prolongation of the QT interval), bradycardia, and/or ventricular tachyarrhythmia (e.g., ventricular fibrillation). In some cases, the drug can safely be administered to patients who otherwise could not tolerate the drug because of such adverse effects. In addition, in some cases, adverse effects of the drug are prevented or diminished by allowing the use of lower dosages of the drug by enhancing or sustaining the efficacy of the drug, as described above.

In some embodiments of the present invention, a method for enhancing or sustaining the efficacy of drug treatment for heart failure comprises administering a drug to a patient and applying signals to a vagus nerve that innervates the heart of the patient. The signals are configured so as to treat the heart failure, which typically results in a synergistic effect between the therapeutic benefit of the drug and the vagal stimulation. Alternatively or additionally, the signals are configured so as to prevent adverse effects sometimes caused by the drug, such as ventricular arrhythmia, idioventricular arrhythmia, premature ventricular contractions, and/or ventricular tachycardia. In addition, in some cases, adverse effects of the drug are prevented or diminished by allowing the use of lower dosages of the drug because of the synergistic effect of the vagal stimulation with the drug treatment.

In some embodiments of the present invention, a method for increasing vagal tone comprises applying signals to the vagus nerve, and configuring the signals to stimulate the vagus nerve, thereby delivering parasympathetic nerve stimulation to the heart, while at the same time minimizing the heart-rate-lowering effects of the stimulation. Such treatment generally results in the beneficial effects of vagal stimulation in clinical situations in which heart rate reduction is not indicated or is contraindicated. For example, such treatment is typically appropriate for heart failure patients who suffer from bradycardia when taking beta-blockers. In addition, such treatment is believed by the inventors to reduce the risk of sudden cardiac death in some patients.

In some embodiments of the present invention, a method for preventing or reducing fibrosis and/or inflammation of the heart comprises applying signals to a vagus nerve that innervates the heart of the patient. Substantially continuous application of such stimulation generally modulates immune system responses, thereby reducing atrial, ventricular, and/or coronary inflammation and/or fibrosis. For some applications, such stimulation is applied for more than about three weeks. Conditions that are believed to be at least partially immune-modulated, and therefore to generally benefit from such vagal stimulation, include, but are not limited to, atrial and ventricular remodeling (e.g., induced by AF, heart failure, myocarditis, and/or myocardial infarct), restenosis, and atherosclerosis.

In some embodiments of the present invention, signals are applied to a vagus nerve of a patient, and the signals are configured to inhibit propagation of naturally-generated efferent action potentials in the vagus nerve. It is hypothesized by the inventors that such inhibition is useful for treating AF, typically by enhancing drug efficacy, and for preventing bradycardia.

In some embodiments of the present invention, electrical signals are applied, typically on a long-term basis, to a vagus nerve of a subject not necessarily suffering from a heart condition, in order to increase the life expectancy, quality of life, and/or healthiness of the subject. Such signals are typically configured to not reduce the heart rate below normal range for a typical human. Such chronic vagal stimulation is hypothesized by the inventors to be effective for increasing life expectancy, quality of life, and/or healthiness by (a) causing a reduction in or prevention of cardiovascular disease and/or events, (b) having an anti-inflammatory effect in the heart or in the rest of the body, (c) reducing the average heart rate, (d) reducing metabolic rate, and/or (e) generally having an anti-stress effect.

In some embodiments of the present invention, apparatus is provided for applying the signals to the vagus nerve, comprising an electrode device and a control unit. The electrode device is applied to a portion of the vagus nerve that innervates the heart of the patient. The control unit drives the electrode device to apply signals to the vagus nerve, and configures the signals based on the desired therapeutic effect, as described above.

In some embodiments of the present invention, apparatus for treating a patient suffering from atrial fibrillation (AF) comprises a control unit and an electrode device, which is applied to a portion of a vagus nerve that innervates the heart of the patient. The control unit drives the electrode device to apply signals to the vagus nerve, and configures the signals to maintain pre-existing AF, i.e., to prevent the return to normal sinus rhythm (NSR). Typically, such pre-existing AF occurred spontaneously in the patient as a disease state, and was not artificially induced (e.g., for treating another heart condition). Alternatively or additionally, AF maintenance is achieved by electrical stimulation of cardiac tissue, such as fat pads, atrial tissue, or pulmonary veins, and/or by administering a drug.

In some embodiments of the present invention, AF is maintained long-term, e.g., longer than about three weeks. Such AF maintenance generally reduces the frequency of recurring transitions between AF and NSR, which transitions are common in patients with AF, particularly in patients with chronic episodic AF. Such repeated transitions are generally undesirable because: (a) they often cause discomfort for the patient, (b) they may increase the risk of thromboembolic events, and (c) they often make prescribing an appropriate drug regimen difficult. Drug regimens that are beneficial for the patient when in AF are often inappropriate when the patient is in NSR, and vice versa. Knowledge that the patient will generally remain in AF typically helps a physician prescribe a more appropriate and/or lower-dosage drug regimen.

In other embodiments of the present invention, AF is maintained short-term, typically between about one day and about three weeks. Such maintenance is generally beneficial during a period in which conventional anticoagulation drug therapy is applied to the patient prior to attempting electrical or pharmacological cardioversion. (Such a period may be desirable when an initial diagnosis of AF occurs more than 48 hours after initiation of AF, or an unknown amount of time after initiation of AF.) Cardioversion is generally not attempted during this period because of the particularly elevated risk of thromboembolic events before the anticoagulation therapy has had time to be effective. AF maintenance to prevent naturally-occurring cardioversion, i.e., reversion to NSR, during this period is believed by the inventors to reduce the risk of thromboembolic events in some patients.

In some embodiments of the present invention, the control unit drives the electrode device to apply signals to the vagus nerve, and configures the signals so as to increase atrial motion. Such increased atrial motion typically causes mixing of the blood in the atrium, which is believed by the inventors to reduce the likelihood of coagulation and resultant thromboembolic events in some patients. Alternatively or additionally, atrial motion is achieved by electrical stimulation of cardiac tissue, such as atrial tissue or fat pads. For some applications, atrial motion is increased using the techniques described herein upon the termination of AF, for example, to prevent or treat electro-mechanical-dissociation (EMD), in which cardiac electrical activity is not coupled with appropriate mechanical contraction.

In other embodiments of the present invention, the control unit drives the electrode device to apply signals to the vagus nerve, and configures the signals so as to restore NSR, i.e., to induce cardioversion. According to a first approach for restoring NSR, the configuration includes repeatedly changing parameters of the stimulation. Such switching of the stimulation in some instances causes fluctuations in the atrial effective refractory period (AERP), thereby breaking reentry cycles and restoring synchronization and NSR. According to a second approach, the control unit (a) paces the heart using conventional pacing techniques, such as by driving a conventional pacemaker to apply pacing signals to the heart, e.g., to the right atrium, right ventricle, or both ventricles, and, simultaneously, (b) configures the signals applied to the vagus nerve to provide generally constant vagal stimulation with a high intensity. The control unit then suddenly ceases vagal stimulation. Such sudden cessation generally destabilizes the atrial cells, resulting in a return to NSR. According to a third approach, typically appropriate for treating AF principally caused by heightened adrenergic tone, the control unit drives the electrode device to apply signals to the vagus nerve, and configures the signals to apply generally constant vagal stimulation, so as to restore NSR.

In some embodiments of the present invention, the apparatus is adapted to be used during conventional electrical atrial defibrillation. The control unit drives the electrode device to apply stimulating signals to the vagus nerve, and configures the stimulating signals to cause severe bradycardia during the defibrillation. Such severe bradycardia generally causes the patient to partially lose consciousness and thereby experience less pain during the defibrillation. The device thus can be thought of as a vagus nerve facilitated tranquilizer. For some applications, the control unit additionally and at generally the same time applies inhibiting signals to the vagus nerve, and configures the inhibiting signals to block vagal pain afferents, thereby further reducing pain experienced by the patient during the defibrillation. In some embodiments, a conventional pacemaker is applied to the heart, and is used to pace the heart in the event of excessive bradycardia caused by the vagal stimulation.

In some embodiments of the present invention, the apparatus comprises a timer and a sensor for detecting AF. When AF is detected, the timer begins a countdown, typically having a duration of between about 24 and 54 hours, such as 48 hours. The apparatus attempts to restore NSR during the countdown, using the cardioversion techniques and apparatus described herein, or methods and apparatus known in the art, such as an implantable defibrillator. Upon completion of the countdown, if NSR has not been successfully restored, the apparatus attempts to maintain AF, typically using techniques described herein. This AF maintenance typically continues until a physician intervenes by signaling the apparatus to terminate maintenance.

In some embodiments of the present invention, the control unit drives the electrode device to (a) apply signals to induce the propagation of efferent action potentials towards the heart, and (b) suppress artificially-induced afferent action potentials towards the brain, in order to minimize any unintended side effect of the signal application. When inducing efferent action potentials towards the heart, the control unit typically drives the electrode device to selectively recruit nerve fibers beginning with smaller-diameter fibers, and to recruit progressively larger-diameter fibers as the desired stimulation level increases. Typically, in order to achieve this smaller-to-larger diameter fiber recruitment order, the control unit stimulates fibers essentially of all diameters using cathodic current from a central cathode, while simultaneously inhibiting fibers in a larger-to-smaller diameter order using anodal current (“efferent anodal current”) from a set of one or more anodes placed between the central cathode and the edge of the electrode device closer to the heart (“the efferent anode set”). Thus, for example, if a small anodal current is applied, then action potentials induced by the cathodic current in the larger diameter fibers are inhibited (because the larger diameter fibers are sensitive to even a small anodal current), while action potentials induced by the cathodic current in smaller fibers are allowed to propagate towards the heart. The amount of parasympathetic stimulation delivered to the heart may generally be increased by decreasing the number of fibers affected by the efferent anodal current, in a smaller-to-larger diameter order, e.g., by decreasing the amplitude or frequency of the efferent anodal current applied to the nerve. Alternatively, the cathodic current is increased in order to increase the parasympathetic stimulation.

The control unit typically suppresses afferent action potentials induced by the cathodic current by inhibiting essentially all or a large fraction of fibers using anodal current (“afferent anodal current”) from a second set of one or more anodes (the “afferent anode set”). The afferent anode set is typically placed between the central cathode and the edge of the electrode device closer to the brain (the “afferent edge”), to block a large fraction of fibers from conveying signals in the direction of the brain during application of the afferent anodal current.

In some embodiments of the present invention, the cathodic current is applied with an amplitude sufficient to induce action potentials in large- and medium-diameter fibers (e.g., A- and B-fibers), but insufficient to induce action potentials in small-diameter fibers (e.g., C-fibers). Simultaneously, an anodal current is applied in order to inhibit action potentials induced by the cathodic current in the large-diameter fibers (e.g., A-fibers). This combination of cathodic and anodal current generally results in the stimulation of medium-diameter fibers (e.g., B-fibers) only. At the same time, a portion of the afferent action potentials induced by the cathodic current are blocked, as described above. By not stimulating large-diameter fibers, such stimulation generally avoids adverse effects sometimes associated with recruitment of such large fibers, such as dyspnea and hoarseness. Stimulation of small-diameter fibers is avoided because these fibers transmit pain sensations and are important for regulation of reflexes such as respiratory reflexes.

In some embodiments of the present invention, the efferent anode set comprises a plurality of anodes. Application of the efferent anodal current in appropriate ratios from the plurality of anodes in these embodiments generally minimizes the “virtual cathode effect,” whereby application of too large an anodal current creates a virtual cathode, which stimulates rather than blocks fibers. When such techniques are not used, the virtual cathode effect generally hinders blocking of smaller-diameter fibers, because a relatively large anodal current is typically necessary to block such fibers, and this same large anodal current induces the virtual cathode effect. Likewise, the afferent anode set typically comprises a plurality of anodes in order to minimize the virtual cathode effect in the direction of the brain.

In some embodiments of the present invention, the current is applied in a series of pulses. The application of the series of pulses in each cardiac cycle typically commences after a variable delay after a detected R-wave, P-wave, or other feature of an ECG. For some applications, other parameters of the applied series of pulses are also varied in real time. Such other parameters include amplitude, number of pulses per trigger (PPT), pulse duration, and pulse repetition interval (i.e., the interval between the leading edges of two consecutive pulses). For some applications, the delay and/or one or more of the other parameters are calculated in real time using a function, the inputs of which include one or more pre-programmed but updateable constants and one or more sensed parameters, such as the R-R interval between cardiac cycles and/or the P-R interval.

Alternatively or additionally, a lookup table of parameters, such as delays and/or other parameters, is used to determine in real time the appropriate parameters for each application of pulses, based on the one or more sensed parameters, and/or based on a predetermined sequence stored in the lookup table. For example, in embodiments of the present invention in which the control unit configures signals applied to the vagus nerve so as to induce cardioversion, such a predetermined sequence may include delays of alternating longer and shorter durations.

In some embodiments of the present invention, the electrical current described herein is applied to a site selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and the current is configured to stimulate autonomic nervous tissue in the site. Alternatively or additionally, the site is selected from the group consisting of: a right ventricle and a right atrium.

The use of at least some of the vagal stimulation techniques described herein may also have the additional beneficial effect of preventing electrical remodeling.

“Vagus nerve,” and derivatives thereof, as used in the present application including the claims, is to be understood to include portions of the left vagus nerve, the right vagus nerve, and branches of the vagus nerve such as the cervical or thoracic vagus nerve, superior cardiac branch, and inferior cardiac branch. Stimulation of the vagus nerve is described herein by way of illustration and not limitation, and it is to be understood that stimulation of other autonomic nerves, including nerves in the epicardial fat pads, a carotid artery, an internal jugular vein, a carotid sinus, a vena cava vein, and/or a pulmonary vein, for treatment of heart conditions or other conditions, is also included within the scope of the present invention.

In some embodiments of the present invention, apparatus for applying vagal stimulation to a patient comprises a control unit and an electrode device, which is applied to a portion of a vagus nerve in order to increase parasympathetic tone of the patient, for example, in order to increase parasympathetic tone with respect to parasympathetic innervation of the heart of the patient. For some applications, the electrode device is applied to a portion of the vagus nerve that innervates the heart. The apparatus is adapted to be used prior to, during, and/or following a clinical procedure. The control unit drives the electrode device to apply vagal stimulation, and typically configures the stimulation to reduce a potential immune-mediated response to the procedure. Such a reduction generally promotes healing after the procedure. When the procedure is heart-related, the vagal stimulation additionally typically (a) reduces mechanical stress by lowering heart rate and pressures, (b) reduces heart rate, and/or (c) improves coronary blood flow.

For some applications, the clinical procedure is selected from one of the following:
coronary artery bypass graft (CABG) surgery;
other bypass graft (such as mesocaval shunting and bypass surgery for peripheral blood flow improvement);
valve replacement surgery;
heart transplantation;
other organ transplantation, such as kidney, liver, skin grafting, and bone marrow transplantation;
percutaneous transluminal coronary angioplasty (PTCA) and/or stenting procedures;
carotid endarterectomy; and
abdominal surgery requiring GI tract anastomosis.

In some embodiments of the present invention, the control unit drives the electrode device to apply vagal stimulation, and configures the stimulation to reduce hyperactivity or activity of brain cells, in order to treat conditions such as stroke and Attention Deficit Hyperactivity Disorder (ADHD). In one application, secondary stroke damage to cells in areas adjacent to the hypoxic area is reduced by reducing the cell activity in these areas. In another application, vagal stimulation is configured to help reduce hyperactivity and improve concentration of a subject suffering from ADHD.

In some embodiments of the present invention, the control unit drives the electrode device to apply vagal stimulation, and configures the stimulation to treat one or more of the following conditions by reducing immune system hyperactivation associated with the condition:
vasculitis, e.g., Wegener granulomatosis, temporal arteritis, Takayasu's arteritis, and/or polyarteritis nodosa;
systemic sclerosis;
systemic lupus erythematosus;
flare of Crohn's disease;
flare of ulcerative colitis;
autoimmune hepatitis;
glomerulonephritis;
arthritis, e.g., reactive or rheumatoid;
pancreatitis;
thyroiditis;
idiopathic thrombocytopenic purpura (ITP);
thrombotic thrombocytopenic purpura (TTP);
multi-organ failure associated with sepsis (especially gram negative sepsis);
anaphylactic shock;
Acute Respiratory Distress Syndrome (ARDS);
asthma;
an allergy or allergic reaction (such as to a drug or body fluid); and
multiple sclerosis.

In some embodiments of the present invention, the control unit drives the electrode device to apply vagal stimulation, and configures the stimulation to treat a habitual behavior or a condition associated with a habitual behavior. The inventors hypothesize that vagal stimulation is effective for treating such behavior because the stimulation interferes with acquired habits or routines of the central nervous system (CNS). For some applications, the control unit drives the electrode device to apply the stimulation at non-constant intervals, such as at random, quasi-random (e.g., generated using a random number generator), or seemingly random intervals (e.g., generated using a preselected set or pattern of varying intervals). The use of such variable intervals breaks cycles of the CNS responsible for such habitual behaviors. The use of non-constant intervals typically reduces the likelihood of the CNS cycle becoming synchronized with the stimulation, i.e., reduces the likelihood of accommodation.

Such habitual behaviors or behavior-related conditions include, but are not limited to:
anorexia, such as anorexia nervosa;
smoking;
drug addiction;
obsessive compulsive disorders;
sleep apnea, e.g., central sleep apnea;
Tourette syndrome; and
hiccups.

In some embodiments of the present invention, the control unit drives the electrode device to apply vagal stimulation that shifts the balance of the autonomic nervous system towards the parasympathetic side thereof, so as to modify the allocation of body resources among different organs and functions. Such vagal stimulation antagonizes the sympathetic system and augments the parasympathetic system, and may be applied in order to treat one or more of the following conditions:
hyperlipidemia—vagal stimulation is applied to promote lipid metabolism and absorption by the liver, and antagonizes carbohydrate-based sympathetically-derived metabolism;
insulin resistance (e.g., type II diabetes)—the sympathetic system generally drives muscle tissue to increase its sensitivity to insulin. Vagal stimulation is applied to augment the parasympathetic system, thereby reducing the short-term sensitivity of muscle tissue to insulin. As a result, the long-term insulin sensitivity of muscle tissue increases;
chronic renal failure—vagal stimulation is applied to increase renal blood flow and glomerular filtration rate (GFR) by reducing blood flow to skeletal muscle (which blood flow is augmented by the sympathetic system), thereby allowing more blood to reach the kidneys, at lower pressures. For some applications, the vagal stimulation is applied while the patient sleeps, or is physically inactive, during which times the need for blood flow to skeletal muscle is reduced. Alternatively or additionally, vagal stimulation increases the GFR by acting on the kidney vascular bed;
chronic hepatic failure—vagal stimulation is applied to increase blood flow through the portal vein by reducing blood flow to skeletal muscle, thereby increasing blood flow through the liver. As a result, a compromised liver is able to perform additional work, and the condition of the patient improves. For some applications, the vagal stimulation is applied while the patient sleeps, or is physically inactive, during which times the need for blood flow to skeletal muscle is reduced;
insomnia—vagal stimulation is applied to shift the autonomic balance towards the parasympathetic system, allowing the mind and body to relax. Vagal stimulation promotes activities such as digestion, relaxation, and sleep;
muscle fatigue (such as associated with heart failure)—vagal stimulation is applied to reduce blood flow and energy consumption of skeletal muscles, thus allowing for muscle rest and recovery (similar to the manner in which beta blockers assist failing hearts);
muscle hypertonia—vagal stimulation is applied to reduce the tension in skeletal muscles, and/or to reduce the symptoms of hypertonia, such as hypertonia associated with upper motor neuron lesions;
sexual dysfunction—vagal stimulation is applied to increase the sensitivity of the sexual organs by increasing parasympathetic input, thereby promoting improved sexual function and/or pleasure;
anemia due to reduced production of red blood cells—vagal stimulation is applied to promote increased medullar red blood cell production and/or extramedullary red blood cell production. In unpublished data obtained from chronically vagal stimulated dogs, the inventors have shown increased extramedullary red blood cell production in response to chronic vagal stimulation; and
reduced peripheral blood flow—in contrast to the sympathetic system that augments blood flow to skeletal muscle, vagal stimulation reduces blood flow to skeletal muscle, thus augmenting the flow in peripheral blood vessels. In addition, parasympathetic stimulation has a direct effect of vasodilatation on peripheral blood vessels, further augmenting peripheral blood flow.

There is therefore provided, in accordance with an embodiment of the present invention, a method including:

identifying that a subject is at risk of suffering from atrial fibrillation (AF); and

responsively to the identifying, reducing a risk of an occurrence of an episode of the AF by:

applying an electrical current to a site of the subject selected from the group consisting of: a vagus nerve, a sinoatrial (SA) node fat pad, a pulmonary vein, a carotid artery, a carotid sinus, a coronary sinus, a vena cava vein, a jugular vein, an azygos vein, an innominate vein, and a subclavian vein, and

configuring the current to stimulate autonomic nervous tissue in the site.

In an embodiment, applying the current includes applying the current even in the absence of a prediction of an imminent episode of the AF. In an embodiment, applying the current includes applying the current in the absence of a prediction of an imminent episode of the AF. In an embodiment, applying the current includes detecting normal sinus rhythm (NSR) of the subject, and applying the current during the detected NSR.

In an embodiment, applying the current does not include configuring the current to achieve a target heart rate or a target heart rate range of the subject.

For some applications, identifying that the subject is at risk includes identifying that the subject suffers from a condition selected from the group consisting of: paroxysmal AF, and self-terminating AF episodes. Alternatively or additionally, identifying that the subject is at risk includes identifying that the subject suffers from at least one condition selected from the group consisting of: an enlarged atrium, multiple atrial premature beats (APBs), mitral stenosis, heart failure, thyrotoxicosis, hypertension, and atrial flutter.

For some applications, identifying includes identifying, after the subject has suffered from at least one episode of the AF, that the subject is at risk. Alternatively, identifying includes identifying, prior to the subject suffering from any known episodes of the AF, that the subject is at risk. Typically, identifying includes identifying by a medical professional that the subject is at risk.

For some applications, applying the current includes configuring the current to delay electrical remodeling of an atrium of the subject, to reduce mechanical stress of a heart of the subject, and/or to induce rhythmic vagal activity.

For some applications, applying the current includes commencing applying at least 24 hours after the identifying.

For some applications, applying the current includes:

applying, during stimulation periods that alternate with rest periods, the current during “on” periods that alternate with low stimulation periods, the “on” periods having on average an “on” duration equal to at least 1 second, and the low stimulation periods having on average a low stimulation duration equal to at least 50% of the “on” duration;

setting the current applied on average during the low stimulation periods to be less than 20% of the current applied on average during the “on” periods;

setting the current applied on average during the rest periods to be less than 20% of the current applied on average during the “on” periods; and

setting the rest periods to have on average a rest period duration equal to at least a cycle duration that equals a duration of a single “on” period plus a duration of a single low stimulation period, and the stimulation periods to have on average a stimulation period duration equal to at least five times the rest period duration.

In an embodiment, applying the current includes:

sensing the occurrence of the episode of the AF; and

responsively to the sensing, configuring the current to reduce a heart rate of the subject.

In an embodiment, applying the current includes applying the current even during the occurrence of the episode of the AF, without configuring the current to resolve the episode.

For some applications, the site includes the sinoatrial (SA) node fat pad, and applying the current includes applying the current to the SA node fat pad.

In an embodiment, applying the current includes detecting whether applying the current causes one or more cardiac contractions, and responsively to finding that applying the current causes the contractions, reducing a strength of the current to a level insufficient to cause the contractions.

In an embodiment, applying the current includes applying the current at least once during each of seven consecutive 48-hour periods. For some applications, applying the current at least once during each of the seven consecutive 48-hour periods includes applying the current at least once during each of 14 consecutive 24-hour periods. For some applications, applying the current at least once during each of the 14 consecutive 24-hour periods includes applying the current at least once during each of 28 consecutive 12-hour periods. For some applications, applying the current includes applying the current in a plurality of pulses, and applying the current at least once during each of the 14 consecutive 24-hour periods includes applying the current in at least 100 of the pulses during each of the 14 consecutive 24-hour periods.

In an embodiment, the site includes the vagus nerve, and applying the current includes applying the current to the vagus nerve. In an embodiment, applying the current includes configuring the current to induce propagation of efferent action potentials traveling towards a heart of the subject, and to suppress artificially-induced afferent action potentials traveling towards a brain of the subject. For some applications, the vagus nerve includes a right vagus nerve, and applying the current includes applying the current to the right vagus nerve.

In an embodiment, applying the current includes configuring the current so as to minimize an effect of the applying of the current on a heart rate of the subject. For some applications, applying the current includes:

setting a threshold heart rate;

sensing the heart rate of the subject;

comparing the sensed heart rate with the threshold heart rate; and

applying the current upon finding that the sensed heart rate is less than the threshold heart rate.

In an embodiment, applying the current includes:

applying the current at a first strength on average;

sensing the occurrence of the episode of the AF; and

responsively to the sensing, applying the current at a second strength on average during a strength reduction period having a duration of at least one minute, which second strength is less than the first strength.

For some applications, applying the current at the second strength includes withholding applying the current. For some applications, applying the current includes, upon a conclusion of the strength reduction period, configuring the current to reduce a heart rate of the subject, upon sensing that the episode of the AF has not terminated and that the subject has an elevated heart rate.

In an embodiment, applying the current includes:

applying the current at a first strength on average;

predicting an imminent episode of the AF; and

responsively to the predicting, applying the current at a second strength on average during a strength reduction period having a duration of at least one minute, which second strength is less than the first strength.

For some applications, applying the current at the second strength includes withholding applying the current.

In an embodiment, identifying includes identifying that the subject is at risk because the subject has undergone an interventional heart procedure. For some applications, the heart procedure includes coronary bypass surgery, and identifying includes identifying that the subject is at risk because the subject has undergone the coronary bypass surgery. For some applications, the heart procedure includes valve replacement surgery, and identifying includes identifying that the subject is at risk because the subject has undergone the valve replacement surgery.

In an embodiment, applying the current includes applying the current in a series of bursts, each of which bursts includes one or more pulses. For some applications, the series of bursts includes at least first and second bursts, the first burst including a plurality of the pulses, and the second burst including at least one of the pulses, and applying the current includes setting (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, applying the current includes:

applying, during “on” periods that alternate with low stimulation periods, at least one of the “on” periods having an “on” duration of at least three seconds, and including at least three of the bursts, and at least one of the low stimulation periods immediately following the at least one of the “on” periods having a low stimulation duration equal to at least 50% of the “on” duration;

setting the current applied on average during the low stimulation periods to be less than 20% of the current applied on average during the “on” periods; and

during at least one transitional period of the at least one of the “on” periods, ramping a number of pulses per burst, the at least one transitional period selected from the group consisting of: a commencement of the at least one of the “on” periods, and a conclusion of the at least one of the “on” periods.

For some applications, applying the current includes synchronizing at least a portion of the bursts with a feature of a cardiac cycle of the subject. For example, the feature of the cardiac cycle may include a P-wave, and applying the current includes synchronizing the at least a portion of the bursts with the P-wave. Alternatively, the feature of the cardiac cycle may include a R-wave, and applying the current includes synchronizing the at least a portion of the bursts with the R-wave.

In an embodiment, applying the current includes: coupling an electrode device to the site; and driving, by a control unit, the electrode device to apply the current. In an embodiment, reducing the risk includes reducing the risk in the absence of a determination by any device directly or indirectly coupled to the control unit that the subject is at risk of suffering from the AF. For some applications, driving includes driving the electrode device to apply the current not responsively to any physiological parameters sensed by any device directly or indirectly coupled to the control unit. For some applications, driving includes driving the electrode device to apply the current not responsively to any measure of a heart rate of the subject determined by the control unit.

There is further provided, in accordance with an embodiment of the present invention, apparatus including:

an electrode device, configured to be coupled to a site of the subject at risk of suffering from atrial fibrillation (AF), the site selected from the group consisting of: a vagus nerve, a sinoatrial (SA) node fat pad, a pulmonary vein, a carotid artery, a carotid sinus, a coronary sinus, a vena cava vein, a jugular vein, an azygos vein, an innominate vein, and a subclavian vein; and

a control unit, configured to reduce a risk of an occurrence of an episode of the AF by:

driving the electrode device to apply an electrical current to the site, and

configuring the current to stimulate autonomic nervous tissue in the site.

There is still further provided, in accordance with an embodiment of the present invention, a method including:

identifying that a subject is at risk of suffering from atrial fibrillation (AF);

responsively to the identifying, delaying electrical remodeling of an atrium of the subject that may be caused by the AF, by:

applying an electrical current to a site of the subject containing parasympathetic nervous tissue, and

configuring the current to stimulate the nervous tissue in the site.

In an embodiment, the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and applying the current includes applying the current to the selected site.

In an embodiment, the site is selected from the group consisting of: the vagus nerve, the epicardial fat pad, the pulmonary vein, the carotid artery, the carotid sinus, the vena cava vein, and the jugular vein, and applying the current includes applying the current to the selected site.

For some applications, delaying the electrical remodeling includes preventing the electrical remodeling of the atrium.

In an embodiment, applying the current includes applying the current even in the absence of a prediction of an imminent episode of the AF. In an embodiment, applying the current includes applying the current in the absence of a prediction of an imminent episode of the AF. In an embodiment, applying the current includes detecting normal sinus rhythm (NSR) of the subject, and applying the current during the detected NSR.

In an embodiment, applying the current does not include configuring the current to achieve a target heart rate or a target heart rate range of the subject.

For some applications, the method includes identifying that the subject suffers from heart failure (HF), and delaying includes, delaying, responsively to the identifying that the subject is at risk of suffering from the AF and that the subject suffers from the HF, the electrical remodeling that may be caused by the AF or by the HF.

Typically, identifying includes identifying by a medical professional that the subject is at risk.

For some applications, delaying includes delaying by administering a drug for treating the AF, responsively to the identifying.

In an embodiment, applying the current includes detecting an episode of the AF, and applying the current responsively to the detecting.

In an embodiment, applying the current includes applying the current not responsively to detecting an episode of the AF.

For some applications, applying the current includes commencing applying at least 24 hours after the identifying.

For some applications, identifying that the subject is at risk includes identifying that the subject suffers from a condition selected from the group consisting of: paroxysmal AF, and self-terminating AF episodes. Alternatively or additionally, identifying that the subject is at risk includes identifying that the subject suffers from at least one condition selected from the group consisting of: an enlarged atrium, multiple atrial premature beats (APBs), mitral stenosis, heart failure, thyrotoxicosis, hypertension, and atrial flutter.

For some applications, identifying includes identifying, after the subject has suffered from at least one episode of the AF, that the subject is at risk. Alternatively, identifying includes identifying, prior to the subject suffering from any known episodes of the AF, that the subject is at risk.

For some applications, applying the current includes configuring the current to reduce mechanical stress of a heart of the subject. For some applications, applying the current includes configuring the current to induce rhythmic vagal activity.

For some applications, applying the current includes:

applying, during stimulation periods that alternate with rest periods, the current during “on” periods that alternate with low stimulation periods, the “on” periods having on average an “on” duration equal to at least 1 second, and the low stimulation periods having on average a low stimulation duration equal to at least 50% of the “on” duration;

setting the current applied on average during the low stimulation periods to be less than 20% of the current applied on average during the “on” periods;

setting the current applied on average during the rest periods to be less than 20% of the current applied on average during the “on” periods; and setting the rest periods to have on average a rest period duration equal to at least a cycle duration that equals a duration of a single “on” period plus a duration of a single low stimulation period, and the stimulation periods to have on average a stimulation period duration equal to at least five times the rest period duration.

For some applications, the site includes a sinoatrial (SA) node fat pad, and applying the current includes applying the current to the SA node fat pad.

For some applications, applying the current includes applying the current even during an episode of the AF, without configuring the current to resolve the episode.

In an embodiment, the site includes the vagus nerve, and applying the current includes applying the current to the vagus nerve. In an embodiment, applying the current includes configuring the current to induce propagation of efferent action potentials traveling towards a heart of the subject, and suppress artificially-induced afferent action potentials traveling towards a brain of the subject. For some applications, the vagus nerve includes a right vagus nerve, and applying the current includes applying the current to the right vagus nerve.

In an embodiment, applying the current includes configuring the current so as to minimize an effect of the applying of the current on a heart rate of the subject. For some applications, applying the current includes:

setting a threshold heart rate;

sensing the heart rate of the subject;

comparing the sensed heart rate with the threshold heart rate; and

applying the current upon finding that the sensed heart rate is less than the threshold heart rate.

In an embodiment, applying the current includes applying the current at least once during each of seven consecutive 48-hour periods. For some applications, applying the current at least once during each of the seven consecutive 48-hour periods includes applying the current at least once during each of 14 consecutive 24-hour periods. For some applications, applying the current at least once during each of the 14 consecutive 24-hour periods includes applying the current at least once during each of 28 consecutive 12-hour periods. For some applications, applying the current includes applying the current in a plurality of pulses, and applying the current at least once during each of the 14 consecutive 24-hour periods includes applying the current in at least 100 of the pulses during each of the 14 consecutive 24-hour periods.

In an embodiment, applying the current includes applying the current during an episode of the AF, and does not include configuring the current to resolve the episode. For some applications, applying the current during the episode includes applying the current during the episode and during at least one period not during the episode. For some applications, applying the current during the episode includes detecting the episode, and applying the current responsively to the detecting.

In an embodiment, applying the current includes:

applying the current at a first strength on average;

sensing an occurrence of an episode of the AF; and

responsively to the sensing, applying the current at a second strength on average during a strength reduction period having a duration of at least one minute, which second strength is less than the first strength.

For some applications, applying the current at the second strength includes withholding applying the current. For some applications, applying the current includes, upon a conclusion of the strength reduction period, configuring the current to reduce a heart rate of the subject, upon sensing that the episode of the AF has not terminated and that the subject has an elevated heart rate.

In an embodiment, applying the current includes:

applying the current at a first strength on average;

predicting an imminent episode of the AF; and

responsively to the predicting, applying the current at a second strength on average during a strength reduction period having a duration of at least one minute, which second strength is less than the first strength.

For some applications, applying the current at the second strength includes withholding applying the current.

In an embodiment, identifying includes identifying that the subject is at risk because the subject has undergone an interventional heart procedure. For some applications, the heart procedure includes coronary bypass surgery, and identifying includes identifying that the subject is at risk because the subject has undergone the coronary bypass surgery. For some applications, the heart procedure includes valve replacement surgery, and identifying includes identifying that the subject is at risk because the subject has undergone the valve replacement surgery.

In an embodiment, applying the current includes applying the current in a series of bursts, each of which bursts includes one or more pulses. For some applications, the series of bursts includes at least first and second bursts, the first burst including a plurality of the pulses, and the second burst including at least one of the pulses, and applying the current includes setting (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, applying the current includes:

applying, during “on” periods that alternate with low stimulation periods, at least one of the “on” periods having an “on” duration of at least three seconds, and including at least three of the bursts, and at least one of the low stimulation periods immediately following the at least one of the “on” periods having a low stimulation duration equal to at least 50% of the “on” duration;

setting the current applied on average during the low stimulation periods to be less than 20% of the current applied on average during the “on” periods; and

during at least one transitional period of the at least one of the “on” periods, ramping a number of pulses per burst, the at least one transitional period selected from the group consisting of: a commencement of the at least one of the “on” periods, and a conclusion of the at least one of the “on” periods.

For some applications, applying the current includes synchronizing at least a portion of the bursts with a feature of a cardiac cycle of the subject. For example, the feature of the cardiac cycle may include a P-wave, and applying the current includes synchronizing the at least a portion of the bursts with the P-wave. Alternatively, the feature of the cardiac cycle may include a R-wave, and applying the current includes synchronizing the at least a portion of the bursts with the R-wave.

In an embodiment, applying the current includes: coupling an electrode device to the site; and driving, by a control unit, the electrode device to apply the current. In an embodiment, reducing the risk includes reducing the risk in the absence of a determination by any device directly or indirectly coupled to the control unit that the subject is at risk of suffering from the AF. For some applications, driving includes driving the electrode device to apply the current not responsively to any physiological parameters sensed by any device directly or indirectly coupled to the control unit. For some applications, driving includes driving the electrode device to apply the current not responsively to any measure of a heart rate of the subject determined by the control unit.

There is additionally provided, in accordance with an embodiment of the present invention, apparatus including:

an electrode device, configured to be coupled to a site of the subject at risk of suffering from atrial fibrillation (AF), the site containing parasympathetic nervous tissue; and

a control unit, configured to delay electrical remodeling of an atrium of the subject that may be caused by the AF, by:

driving the electrode device to apply an electrical current to the site, and

configuring the current to stimulate the nervous tissue in the site.

There is yet additionally provided, in accordance with an embodiment of the present invention, a method including:

applying an electrical current, at a first strength on average, to a site of a subject containing parasympathetic nervous tissue;

configuring the current to stimulate the nervous tissue in the site;

performing at least one action selected from the group consisting of: sensing an occurrence of an episode of atrial fibrillation (AF), and predicting an imminent episode of the AF; and

responsively to the performing, applying the current at a second strength on average during a strength reduction period having a duration of at least one minute, which second strength is less than the first strength.

In an embodiment, the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and applying the current includes applying the current to the selected site.

In an embodiment, performing includes sensing the occurrence of the episode of the AF, and applying the current at the second strength includes during the strength reduction period includes applying the current at the second strength during the episode.

In an embodiment, performing includes predicting the imminent episode of the AF.

For some applications, the method includes, upon a conclusion of the strength reduction period, configuring the current to reduce a heart rate of the subject, upon sensing that the episode of the AF has not terminated and that the subject has an elevated heart rate.

For some applications, applying the current at the second strength includes withholding applying the current.

For some applications, the strength reduction period has a duration of at least one minute, and applying the current at the second strength on average includes applying the current at the second strength on average during the strength reduction period having the duration of at least one minute.

For some applications, the method includes identifying that the subject is at risk of suffering from AF, and applying the current at the first strength includes, responsively to the identifying, reducing a risk of the occurrence of the episode of the AF by applying the current at the first strength.

For some applications, applying the current at the first strength includes applying the current at the first strength at least once during each of seven consecutive 48-hour periods.

For some applications, identifying that the subject is at risk includes identifying that the subject suffers from a condition selected from the group consisting of: paroxysmal AF, self-terminating AF episodes, an enlarged atrium, multiple atrial premature beats (APBs), mitral stenosis, heart failure, thyrotoxicosis, hypertension, and atrial flutter. Typically, identifying includes identifying by a medical professional that the subject is at risk.

In an embodiment, applying the current at the first strength includes applying the current at the first strength even in the absence of a prediction of an imminent episode of the AF. In an embodiment, applying the current at the first strength includes applying the current at the first strength in the absence of a prediction of an imminent episode of the AF. In an embodiment, applying the current at the first strength includes detecting normal sinus rhythm (NSR) of the subject, and applying the current at the first strength during the detected NSR.

In an embodiment, applying the current at the first strength does not include configuring the current to achieve a target heart rate or a target heart rate range of the subject.

for some applications, applying the current at the first strength includes commencing applying at least 24 hours after the identifying.

For some applications, applying the current at the first strength includes:

applying, during stimulation periods that alternate with rest periods, the current during “on” periods that alternate with low stimulation periods, the “on” periods having on average an “on” duration equal to at least 1 second, and the low stimulation periods having on average a low stimulation duration equal to at least 50% of the “on” duration;

setting the current applied on average during the low stimulation periods to be less than 20% of the current applied on average during the “on” periods;

setting the current applied on average during the rest periods to be less than 20% of the current applied on average during the “on” periods; and

setting the rest periods to have on average a rest period duration equal to at least a cycle duration that equals a duration of a single “on” period plus a duration of a single low stimulation period, and the stimulation periods to have on average a stimulation period duration equal to at least five times the rest period duration.

For some applications, the site includes a sinoatrial (SA) node fat pad, and applying the current at the first strength includes applying the current to the SA node fat pad.

In an embodiment, the site includes the vagus nerve, and applying the current at the first strength includes applying the current to the vagus nerve. In an embodiment, applying the current at the first strength includes configuring the current to induce propagation of efferent action potentials traveling towards a heart of the subject, and suppress artificially-induced afferent action potentials traveling towards a brain of the subject.

In an embodiment, applying the current at the first strength includes configuring the current so as to minimize an effect of the applying of the current on a heart rate of the subject. For some applications, applying the current at the first strength includes:

setting a threshold heart rate;

sensing the heart rate of the subject;

comparing the sensed heart rate with the threshold heart rate; and

applying the current at the first strength upon finding that the sensed heart rate is less than the threshold heart rate.

In an embodiment, applying the current at the first strength includes applying the current in a series of bursts, each of which bursts includes one or more pulses. For some applications, the series of bursts includes at least first and second bursts, the first burst including a plurality of the pulses, and the second burst including at least one of the pulses, and applying the current at the first strength includes setting (a) a pulse repetition interval (PRI) of the first burst to be on average at least 20 ms, (b) an interburst interval between initiation of the first burst and initiation of the second burst to be less than 10 seconds, (c) an interburst gap between a conclusion of the first burst and the initiation of the second burst to have a duration greater than the average PRI, and (d) a burst duration of the first burst to be less than a percentage of the interburst interval, the percentage being less than 67%.

For some applications, applying the current at the first strength includes:

applying, during “on” periods that alternate with low stimulation periods, at least one of the “on” periods having an “on” duration of at least three seconds, and including at least three of the bursts, and at least one of the low stimulation periods immediately following the at least one of the “on” periods having a low stimulation duration equal to at least 50% of the “on” duration;

setting the current applied on average during the low stimulation periods to be less than 20% of the current applied on average during the “on” periods; and

during at least one transitional period of the at least one of the “on” periods, ramping a number of pulses per burst, the at least one transitional period selected from the group consisting of: a commencement of the at least one of the “on” periods, and a conclusion of the at least one of the “on” periods.

In an embodiment, applying the current at the first strength includes synchronizing at least a portion of the bursts with a feature of a cardiac cycle of the subject. For example, the feature of the cardiac cycle may include a P-wave, and applying the current at the first strength includes synchronizing the at least a portion of the bursts with the P-wave. Alternatively, the feature of the cardiac cycle may include a R-wave, and applying the current at the first strength includes synchronizing the at least a portion of the bursts with the R-wave.

There is also provided, in accordance with an embodiment of the present invention, apparatus including:

an electrode device, configured to be coupled to a site of the subject at risk of suffering from atrial fibrillation (AF), the site containing parasympathetic nervous; and

a control unit, configured to:

drive the electrode device to apply an electrical current to the site at a first strength on average,

configure the current to stimulate the nervous tissue in the site,

perform at least one action selected from the group consisting of: sensing an occurrence of an episode of atrial fibrillation (AF), and predicting an imminent episode of the AF, and

responsively to the performance, apply the current at a second strength on average during a strength reduction period having a duration of at least one minute, which second strength is less than the first strength.

There is further provided, in accordance with an embodiment of the present invention, a method including:

identifying that a subject is at risk of suffering from atrial fibrillation (AF); and

responsively to the identifying, reducing a risk of an occurrence of an episode of the AF by:

coupling an electrode device to a site of a subject containing parasympathetic nervous tissue,

driving, by a control unit, the electrode device to apply an electrical current to the site not responsively to any physiological parameters sensed by any device directly or indirectly coupled to the control unit, and

configuring the current to stimulate autonomic nervous tissue in the site.

In an embodiment, the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and applying the current includes applying the current to the selected site.

In an embodiment, driving includes driving the electrode device to apply the current at least once during each of seven consecutive 48-hour periods.

In an embodiment, the site includes the vagus nerve, and applying the current includes applying the current to the vagus nerve. In an embodiment, configuring includes configuring the current to induce propagation of efferent action potentials traveling towards a heart of the subject, and suppress artificially-induced afferent action potentials traveling towards a brain of the subject.

There is still further provided, in accordance with an embodiment of the present invention, apparatus including:

an electrode device, configured to be coupled to a site of the subject at risk of suffering from atrial fibrillation (AF), the site containing parasympathetic nervous tissue; and

a control unit, configured to reduce a risk of an occurrence of an episode of the AF by:

driving the electrode device to apply an electrical current to the site not responsively to any physiological parameters sensed by any device directly or indirectly coupled to the control unit, and

configuring the current to stimulate the nervous tissue in the site.

There is additionally provided, in accordance with an embodiment of the present invention, a method including:

setting a threshold heart rate;

sensing a heart rate of a subject;

comparing the sensed heart rate with the threshold heart rate;

upon finding that the sensed heart rate is less than the threshold heart rate, applying a current to a site of the subject containing parasympathetic nervous tissue; and

configuring the current to increase vagal tone of the subject by stimulating the nervous tissue in the site, and to minimize an effect of the applying of the current on a heart rate of the subject.

In an embodiment, the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, a subclavian vein, a right ventricle, and a right atrium, and applying the current includes applying the current to the selected site.

In an embodiment, setting the threshold heart rate includes setting the threshold heart rate to a percentage of a normal heart rate for the subject. Alternatively, setting the threshold heart rate includes setting the threshold heart rate to a percentage of a normal heart rate for typical subjects.

There is yet additionally provided, in accordance with an embodiment of the present invention, apparatus including:

an electrode device, configured to be coupled to a site of a subject containing parasympathetic nervous tissue; and

a control unit, configured to:

store a threshold heart rate,

sense a heart rate of the subject,

compare the sensed heart rate to the threshold heart rate, and

upon finding that that the sensed heart rate is less than the threshold heart rate, drive the electrode device to apply a current to the site, and to configure the current to (a) increase vagal tone of the subject by stimulating the nervous tissue in the site, and (b) minimize an effect of the applying of the current on a heart rate of the subject.

There is also provided, in accordance with an embodiment of the present invention, a method including:

identifying that a subject is at risk of suffering from atrial fibrillation (AF); and

responsively to the identifying, reducing a risk of an occurrence of an episode of the AF by:

detecting normal sinus rhythm (NSR) of the subject,

during the detected NSR, applying an electrical current to a site of the subject

containing parasympathetic nervous tissue, and configuring the current to stimulate the nervous tissue in the site.

In an embodiment, the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and applying the current includes applying the current to the selected site.

In an embodiment, applying the current includes configuring the current so as to minimize an effect of the applying of the current on a heart rate of the subject.

For some applications, applying the current includes:

setting a threshold heart rate;

sensing the heart rate of the subject;

comparing the sensed heart rate with the threshold heart rate; and

applying the current upon finding that the sensed heart rate is less than the threshold heart rate.

For some applications, applying the current includes:

applying the current at a first strength on average;

sensing the occurrence of the episode of the AF; and

responsively to the sensing, applying the current at a second strength on average during a strength reduction period having a duration of at least one minute, which second strength is less than the first strength.

For some applications, applying the current at the second strength includes withholding applying the current. For some applications, applying the current includes, upon a conclusion of the strength reduction period, configuring the current to reduce a heart rate of the subject, upon sensing that the episode of the AF has not terminated and that the subject has an elevated heart rate.

For some applications, applying the current includes:

applying the current at a first strength on average;

predicting that the occurrence of the episode of the AF is imminent; and

responsively to the predicting, applying the current at a second strength on average during a strength reduction period having a duration of at least one minute, which second strength is less than the first strength.

For some applications, applying the current at the second strength includes withholding applying the current.

There is further provided, in accordance with an embodiment of the present invention, apparatus including:

an electrode device, configured to be coupled to a site of a subject at risk of suffering from atrial fibrillation (AF), the site containing parasympathetic nervous tissue; and
a control unit, configured to reduce a risk of an occurrence of an episode of the AF by:

detecting normal sinus rhythm (NSR) of the subject,

during the detected NSR, driving the electrode device to apply an electrical current to the site, and

configuring the current to stimulate the nervous tissue in the site.

There is further provided, in accordance with an embodiment of the present invention, a method for treating a subject suffering from atrial fibrillation, including:

applying a current to a site of the subject selected from the group consisting of: a vagus nerve of the subject, an epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

configuring the current to increase vagal tone of the subject, and to minimize an effect of the applying of the current on a heart rate of the subject, so as to treat the condition.

In an embodiment, the method includes applying a pacing signal to a heart of the subject in conjunction with applying the current to the site.

In an embodiment, the method includes sensing a heart rate of the subject, and configuring the current includes configuring the current using a feedback loop, an input of which is the sensed heart rate.

There is further provided, in accordance with an embodiment of the present invention, a method for treating a subject suffering from a condition, including:

applying a current to a site of the subject selected from the group consisting of: a vagus nerve of the subject, and epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

configuring the current so as to delay electrical remodeling of an atrium of the subject caused by the condition.

In an embodiment, configuring the current includes configuring the current so as to prevent electrical remodeling of the atrium caused by the condition.

In an embodiment, the condition includes heart failure (HF), and configuring the current includes configuring the current so as to prevent the electrical remodeling caused by the HF.

In an embodiment, the condition includes both atrial fibrillation (AF) and heart failure (HF), and configuring the current includes configuring the current so as to prevent the electrical remodeling caused by the AF and the HF.

In an embodiment, the method includes administering a drug for treating the condition.

In an embodiment, no drug is administered for treating the condition during a period beginning about 24 hours before initiation of application of the current and ending upon the initiation of the application of the current.

In an embodiment, the condition includes atrial fibrillation (AF), and configuring the current includes configuring the current so as to prevent the electrical remodeling caused by the AF. For some applications, applying the current includes detecting an occurrence of the AF, and applying the current responsively to the detecting. For some applications, applying the current includes applying the current not responsively to detecting an occurrence of the AF.

There is yet additionally provided, in accordance with an embodiment of the present invention, a method including:

applying a current to a site of a subject selected from the group consisting of: a vagus nerve of the subject, an epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

configuring the current to reduce mechanical tension on at least one atrium of the subject, so as to reduce a risk of an occurrence of atrial fibrillation (AF).

In an embodiment, the method includes administering to the subject a drug for treating the AF.

There is still further provided, in accordance with an embodiment of the present invention, a method for treating a subject, including:

applying a current to a site of the subject selected from the group consisting of: a vagus nerve of the subject, and epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

configuring the current so as to have an antiarrhythmic effect on an atrium of the subject.

For some applications, the site includes a right vagus nerve of the subject, and applying the current includes applying the current to the right vagus nerve.

In an embodiment, the method includes administering an antiarrhythmic drug to the subject in conjunction with applying the current.

For some applications, configuring the current includes configuring the current so as to induce rhythmic vagal activity in the subject.

In an embodiment, applying the current includes applying the current to the site intermittently during alternating “on” and “off” periods. For some applications, applying the current intermittently includes setting each of the “on” periods to have a duration of between about 1 and about 15 seconds, and each of the “off” periods to have a duration of between about 5 and about 20 seconds.

In an embodiment, the site includes the vagus nerve, and applying the current includes applying the current to the vagus nerve. For some applications, applying the current includes applying a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set. For some applications, applying the current includes applying a stimulating current, which is capable of inducing action potentials in the vagus nerve, and an inhibiting current, which is capable of inhibiting action potentials induced by the stimulating current and traveling in the vagus nerve in an afferent direction toward a brain of the subject.

In an embodiment, applying the current includes applying the current in respective bursts of pulses in each of a plurality of cardiac cycles of the subject. For some applications, applying the current includes applying a first pulse of each of the bursts after a delay from a sensed feature of an electrocardiogram (ECG) of the subject.

In an embodiment, the method includes sensing a physiological parameter of the subject, and configuring the current includes configuring the current at least in part responsively to the sensed physiological parameter. For some applications, sensing the physiological parameter includes sensing a heart rate of the subject.

In an embodiment, configuring the current includes configuring the current so as to minimize an effect of the applying of the current on a heart rate of the subject.

In an embodiment, applying the current includes applying the current in respective bursts of pulses in each of a plurality of cardiac cycles of the subject. For some applications, applying the current includes applying the current to a left vagus nerve of the subject. For some applications, applying the current includes configuring each of the pulses to have a duration of between about 200 microseconds and about 2.5 milliseconds. For some applications, applying the current includes configuring each of the pulses to have a duration of between about 2.5 and about 5 milliseconds. For some applications, applying the current includes configuring each of the bursts to have a duration of between about 0.2 and about 40 milliseconds. For some applications, applying the current includes configuring each of the bursts to contain between about 1 and about 10 pulses. For some applications, applying the current includes configuring the pulses within each of the bursts to have a pulse repetition interval of between about 2 and about 10 milliseconds. For some applications, applying the current includes configuring the pulses to have an amplitude of between about 0.5 and about 5 mA. For some applications, applying the current includes applying the bursts less than every heartbeat of the subject. For some applications, applying the current includes applying the bursts once per heartbeat of the subject. For some applications, applying the current includes applying the current to the site intermittently during alternating “on” and “off” periods, each of the “on” periods having a duration of at least about 1 second. For some applications, applying the current includes applying each of the bursts after a variable or fixed delay following a P-wave of the subject. For some applications, the delay has a duration equal to less than about 50 ms, while for other applications the delay has a duration equal to between about two-thirds and about 90% of a duration of a cardiac cycle of the subject. For some applications, applying the current includes substantially continuously measuring the duration of the cardiac cycle.

In an embodiment, applying the current includes applying the current in respective bursts of pulses in each of a plurality of cardiac cycles of the subject. For some applications, applying the current includes configuring each of the pulses to have a duration of between about 100 microseconds and about 2.5 milliseconds. For some applications, applying the current includes configuring each of the bursts to have a duration of between about 1 and about 180 milliseconds. For some applications, applying the current includes configuring each of the bursts to contain between about 1 and about 10 pulses. For some applications, applying the current includes configuring the pulses within each of the bursts to have a pulse repetition interval of between about 1 and about 20 milliseconds. For some applications, applying the current includes configuring the pulses to have an amplitude of between about 0.1 and about 9 mA. For some applications, applying the current includes applying the bursts once every second heartbeat. For some applications, applying the current includes applying the bursts once every third heartbeat. For some applications, applying the current includes applying the current to the site intermittently during alternating “on” and “off” periods, each of the “on” periods having a duration of at least about 1 second. For some applications, applying the current includes applying each of the bursts after a delay following an R-wave of the subject, the delay having a duration of about 100 milliseconds.

In an embodiment, applying the current includes applying the current in respective bursts of between about 1 and about 10 pulses in each of a plurality of cardiac cycles of the subject, and applying a first pulse of each of the bursts after a delay of about 100 milliseconds after a sensed R-wave of an electrocardiogram (ECG) of the subject.

For some applications, applying the current includes configuring each of the bursts to contain about three pulses. For some applications, applying the current includes varying a number of the pulses in each of the bursts responsive to a sensed parameter of a respiratory cycle of the subject. For some applications, applying the current includes varying a number of the pulses in each of the bursts responsive to a sensed heart rate of the subject. For some applications, the site includes the vagus nerve, and applying the current includes applying the current to the vagus nerve, and, responsive to a sensed heart rate of the subject, varying a number of nerve fibers of the vagus nerve that are recruited.

For some applications, the site includes the vagus nerve, and applying the current includes applying the current to the vagus nerve, and, responsive to a sensed parameter of a respiratory cycle of the subject, varying a number of nerve fibers of the vagus nerve that are recruited. For some applications, applying the current includes cycling between a first set of parameters and a second set of parameters. For some applications, cycling includes applying each set of parameters for less than about 15 seconds. For some applications, cycling includes applying each set of parameters for between about 1 and about 4 seconds. For some applications, the first set of parameters includes a first amplitude, the second set of parameters includes a second amplitude, greater than the first amplitude, and applying the current includes varying a number of nerve fibers of the vagus nerve that are recruited by cycling between the first set of parameters and the second set of parameters.

For some applications, cycling includes synchronizing application of the first set of parameters with inhalation by the subject, and synchronizing application of the second set of parameters with exhalation by the subject. For some applications, at least one of the first and second sets of parameters includes a pulse repetition interval of between about 4 and about 20 milliseconds, and applying the current includes cycling between the first and second sets of parameters. For some applications, at least one of the first and second sets of parameters includes a pulse width of between about 0.1 and about 2 milliseconds, and applying the current includes cycling between the first and second sets of parameters. For some applications, the first set of parameters includes application of the current at one pulse per each of the bursts, the second set of parameters includes application of the current at about three pulses per each of the bursts, and applying the current includes cycling between the first and second sets of parameters.

There is still further provided, in accordance with an embodiment of the present invention, apparatus for treating a subject suffering from a condition, including:

an electrode device, adapted to be coupled to a site of the subject selected from the group consisting of: a vagus nerve of the subject, an epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

a control unit, adapted to:

drive the electrode device to apply an electrical current to the site, and

configure the current so as to delay electrical remodeling of an atrium of the subject caused by the condition.

There is also provided, in accordance with an embodiment of the present invention, apparatus including:

an electrode device, adapted to be coupled to a site of a subject selected from the group consisting of: a vagus nerve of the subject, an epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

a control unit, adapted to:

drive the electrode device to apply an electrical current to the site, and

configure the current to reduce mechanical tension on at least one atrium of the subject, so as to reduce a risk of an occurrence of atrial fibrillation (AF).

There is still further provided, in accordance with an embodiment of the present invention, apparatus for treating a subject, including:

an electrode device, adapted to be coupled to a site of the subject selected from the group consisting of: a vagus nerve of the subject, an epicardial fat pad of the subject, a pulmonary vein of the subject, a carotid artery of the subject, a carotid sinus of the subject, a vena cava vein of the subject, a jugular vein of the subject, an azygos vein of the subject, an innominate vein of the subject, and a subclavian vein of the subject; and

a control unit, adapted to:

drive the electrode device to apply an electrical current to the site, and

configure the current so as to have an antiarrhythmic effect on an atrium of the subject.

There is additionally provided, in accordance with an embodiment of the present invention, a method for treating a subject suffering from atrial fibrillation (AF), including:

administering a drug for treating the AF to the subject;

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current to increase vagal tone of the subject, so as to treat the AF.

In an embodiment, configuring the current includes configuring the current so as to enhance an efficacy of the drug.

In an embodiment, the method includes detecting an occurrence of the AF, and applying the current includes applying the current responsive to the detecting of the occurrence.

In an embodiment, administering the drug includes administering the drug at a dosage determined independently of applying the current.

In an embodiment, administering the drug includes administering the drug at a dosage lower than a dosage determined independently of applying the current.

In an embodiment, the subject additionally suffers from heart failure (HF), and the method includes administering a HF drug for treating the HF of the subject, and configuring the current includes configuring the current so as to enhance an efficacy of the HF drug.

For some applications, configuring the current so as to enhance the efficacy of the drug includes configuring the current so as to prevent electrical remodeling of at least one atrium of the subject. Alternatively or additionally, configuring the current so as to enhance the efficacy of the drug includes configuring the current so as to delay electrical remodeling of at least one atrium of the subject.

In an embodiment, configuring the current so as to enhance the efficacy of the drug includes configuring the current so as to achieve a therapeutic effect similar to that of the drug.

In an embodiment, configuring the current so as to enhance the efficacy of the drug includes configuring the current so as to reduce a QT interval of an electrocardiogram (ECG) of the subject.

In an embodiment, administering the drug includes administering a beta-blocker.

In an embodiment, administering the drug includes administering a sinus rhythm maintenance drug. For some applications, configuring the current so as to enhance the efficacy of the drug includes configuring the current so as to increase vagal tone of the subject. For some applications, configuring the current so as to enhance the efficacy of the drug includes configuring the current so as to reduce an atrial effective refractory period of the subject. For some applications, configuring the current so as to enhance the efficacy of the drug includes configuring the current so as to have an antiarrhythmic effect on an atrium of the subject. For some applications, configuring the current so as to enhance the efficacy of the drug includes configuring the current to reduce mechanical tension on at least one atrium of the subject.

For some applications, administering the sinus rhythm maintenance drug includes administering a beta-blocker. Alternatively or additionally, administering the sinus rhythm maintenance drug includes administering quinidine. Further alternatively or additionally, administering the sinus rhythm maintenance drug includes administering a drug selected from the list consisting of: digoxin, amiodarone, disopyramide, dofetilide, a class IC drug, procainamide, and sotalol.

For some applications, the method includes applying conventional cardioversion to the subject so as to treat the AF. For some applications, configuring the current so as to enhance the efficacy of the drug includes configuring the current so as to induce rhythmic vagal activity in the subject.

In an embodiment, administering the drug includes administering a ventricular rate control drug. For some applications, configuring the current so as to enhance the efficacy of the drug includes configuring the current so as to control a ventricular response rate of the subject. For some applications, configuring the current so as to enhance the efficacy of the drug includes configuring the current so as to improve cardiac output of the subject.

For some applications, administering the ventricular rate control drug includes administering a beta-blocker. Alternatively or additionally, administering the ventricular rate control drug includes administering a drug selected from the list consisting of: a calcium channel antagonist and digoxin.

In an embodiment, administering the drug includes administering an antithrombotic drug. For some applications, administering the antithrombotic drug includes administering an anticoagulation drug that inhibits a coagulation cascade. Alternatively or additionally, administering the antithrombotic drug includes administering a drug that inhibits platelet aggregation. For some applications, configuring the current so as to enhance the efficacy of the antithrombotic drug includes configuring the current so as to increase atrial motion of the subject. For some applications, administering the antithrombotic drug includes selecting a dosage of the drug to achieve a target international normalized ratio (INR) lower than a target INR determined independently of applying the current. For some applications, configuring the current so as to enhance the efficacy of the drug includes configuring the current so as to induce rhythmic vagal activity in the subject.

There is also provided, in accordance with an embodiment of the present invention, a method for treating a subject suffering from heart failure (HF), including:

administering a drug for treating the HF to the subject;

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to enhance an efficacy of the drug.

In an embodiment, configuring the current so as to enhance the efficacy of the drug includes configuring the current so as to treat the HF.

For some applications, configuring the current so as to enhance the efficacy of the drug includes configuring the current to inhibit propagation of naturally-generated efferent action potentials traveling through the site.

In an embodiment, administering the drug includes administering a positive inotropic drug. For some applications, administering the positive inotropic drug includes administering a positive inotropic drug selected from the list consisting of: digoxin, dopamine, dobutamine, adrenaline, aminone, and milrinone.

In an embodiment, administering the drug includes administering a preload reduction drug. For some applications, administering the preload reduction drug includes administering a preload reduction drug selected from the list consisting of: an ACE inhibitor, a nitrate, and sodium nitroprusside. For some applications, configuring the current so as to enhance the efficacy of the preload reduction drug includes configuring the current so as to decrease atrial contractile force of a heart of the subject. For some applications, applying the current includes applying the current to the site intermittently during alternating “on” and “off” periods. For some applications, applying the current intermittently includes setting each of the “on” periods to have a duration of between about 1 and about 15 seconds, and each of the “off” periods to have a duration of between about 5 and about 20 seconds.

There is further provided, in accordance with an embodiment of the present invention, a method for treating a subject suffering from a condition, including:

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current to increase vagal tone of the subject, and to minimize an effect of the applying of the current on a heart rate of the subject, so as to treat the condition.

In an embodiment, the condition is selected from the list consisting of: atrial fibrillation, heart failure, atherosclerosis, restenosis, myocarditis, cardiomyopathy, post-myocardial infarct remodeling, and hypertension, and configuring the current includes configuring the current so as to treat the selected condition. Alternatively or additionally, the condition is selected from the list consisting of: obesity, constipation, irritable bowl syndrome, rheumatoid arthritis, glomerulonephritis, an autoimmune disease, multiple sclerosis, hepatitis, pancreatitis, portal vein hypertension, thyroiditis, type I diabetes, and type II diabetes, and configuring the current includes configuring the current so as to treat the selected condition.

For some applications, configuring the current includes configuring the current so as to reduce a risk of sudden cardiac death of the subject.

For some applications, applying the current includes applying the current substantially only at nighttime. For some applications, applying the current includes applying the current during a daytime period and during a nighttime period, the applying during the nighttime period being longer than the applying during the daytime period.

For some applications, applying the current includes detecting exercise by the subject, and applying the current responsively to the detecting.

For some applications, applying the current to the site of the subject includes selecting a subject that is receiving a heart-rate lowering drug, and who has achieved a heart rate within a desired range prior to initiation of applying the current.

For some applications, applying the current to the site of the subject includes selecting a subject who experiences, when the heart rate is reduced, a symptom selected from the list consisting of: discomfort, and a reduction in exercise capacity.

For some applications, applying the current to the site of the subject includes selecting a subject who has a tendency towards bradycardia when receiving vagal stimulation that is not configured to minimize an effect thereof on the heart rate.

For some applications, the condition includes low cardiac output, and configuring the current includes configuring the current so as to treat the low cardiac output. For some applications, the condition includes acute myocardial infarction with cardiogenic shock, and configuring the current includes configuring the current so as to treat the acute myocardial infarction. For some applications, the condition includes heart failure and beta-blocker-induced bradycardia, and configuring the current includes configuring the current so as to treat the heart failure and bradycardia.

In an embodiment, the method includes applying a pacing signal to a heart of the subject in conjunction with applying the current to the site.

In an embodiment, the method includes sensing a heart rate of the subject, and configuring the current includes configuring the current using a feedback loop, an input of which is the sensed heart rate.

There is still further provided, in accordance with an embodiment of the present invention, a method for treating a subject suffering from a condition, including:

administering to the subject a drug for treating the condition;

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to reduce an adverse effect sometimes caused by the drug.

In an embodiment, the condition includes atrial fibrillation (AF), administering the drug includes administering a drug for treating the AF, and configuring the current includes configuring the current so as to reduce the adverse effect sometimes caused by the AF drug.

In an embodiment, the condition includes heart failure (HF), administering the drug includes administering a drug for treating the HF, and configuring the current includes configuring the current so as to reduce the adverse effect sometimes caused by the HF drug.

In an embodiment, the condition includes an emergency condition, and administering the drug includes administering atropine.

In an embodiment, the adverse effect includes idioventricular arrhythmia, and configuring the current includes configuring the current so as to reduce the idioventricular arrhythmia. In an embodiment, the adverse effect includes premature ventricular contractions, and configuring the current includes configuring the current so as to reduce the premature ventricular contractions. In an embodiment, the adverse effect includes ventricular tachycardia, and configuring the current includes configuring the current so as to reduce the ventricular tachycardia.

In an embodiment, the adverse effect includes ventricular arrhythmia, and configuring the current includes configuring the current so as to reduce the ventricular arrhythmia. For some applications, configuring the current includes configuring the current so as to induce rhythmic vagal activity in the subject.

In an embodiment, administering the drug includes administering the drug at a dosage lower than a usual dosage determined independently of applying the current, and configuring the current includes configuring the current so as to enhance an efficacy of the drug to a degree that the lower dosage has substantially the same efficacy as the usual dosage. For some applications, administering the drug includes administering digoxin at the lower dosage.

In an embodiment, the adverse effect includes ventricular tachyarrhythmia, and configuring the current includes configuring the current so as to reduce the ventricular tachyarrhythmia. For some applications, the ventricular tachyarrhythmia includes ventricular fibrillation, and configuring the current includes configuring the current so as to reduce the ventricular fibrillation. For some applications, administering the drug includes administering a drug selected from the list consisting of: an antiarrhythmic drug, and a positive inotropic drug.

In an embodiment, the adverse effect includes a repolarization abnormality, and configuring the current includes configuring the current so as to reduce the repolarization abnormality. For some applications, the repolarization abnormality includes a prolongation of a QT interval of the subject, and configuring the current includes configuring the current so as to reduce the prolongation of the QT interval.

In an embodiment, administering the drug includes administering the drug at a dosage greater than a dosage determined independently of applying the current, and configuring the current so as to reduce the adverse effect includes configuring the current so as to reduce an adverse effect sometimes caused by the greater dosage. For some applications, administering the drug includes administering a class IC drug.

In an embodiment, administering the drug includes administering a positive inotropic agent for a period of time having a duration greater than about one day. For some applications, administering the positive inotropic agent includes administering the positive inotropic agent for a period having a duration greater than about 7 days. For some applications, administering the positive inotropic agent includes administering a positive inotropic agent other than digitalis. For some applications, the adverse effect is selected from the list consisting of: a chronotropic effect of the positive inotropic agent, and a proarrhythmic effect of the positive inotropic agent, and configuring the current includes configuring the current so as to reduce the selected adverse effect. For some applications, the subject is in a stable condition, and administering the positive inotropic agent includes administering the positive inotropic agent to the stable subject.

In an embodiment, the adverse effect includes an occurrence of bradycardia, and configuring the current includes configuring the current so as to reduce the occurrence of bradycardia. For some applications, configuring the current includes configuring the current to inhibit propagation of naturally-generated efferent action potentials traveling through the site, so as to reduce the bradycardia. For some applications, applying the current includes detecting the occurrence of bradycardia, and terminating applying the current responsive to the detecting. For some applications, applying the current includes detecting the occurrence of bradycardia, and reducing an intensity of the current responsive to the detecting. For some applications, the method includes detecting the occurrence of bradycardia, and, responsive to the detecting, applying a pacing signal to a heart of the subject.

There is additionally provided, in accordance with an embodiment of the present invention, a method for treating a subject, including:

applying a current to a site of the subject selected from the list consisting of: a vagus nerve of the subject, and epicardial fat pad of the subject, a pulmonary vein of the subject, a carotid artery of the subject, a carotid sinus of the subject, a vena cava vein of the subject, and an internal jugular vein of the subject; and

configuring the current so as to reduce a heart condition of the subject selected from the list consisting of: fibrosis of the heart, and inflammation of the heart.

For some applications, applying the current includes substantially continuously applying the current. For some applications, applying the current includes applying the current during an application period lasting at least about three weeks, and configuring the current such that, during the application period, a longest duration of time in which no current is applied is less than four hours. For some applications, applying the current includes applying the current for a period having a duration of more than about three weeks.

For some applications, the heart condition includes the fibrosis of the heart, and configuring the current includes configuring the current so as to reduce the fibrosis. Alternatively or additionally, the heart condition includes the inflammation of the heart, and configuring the current includes configuring the current so as to reduce the inflammation.

There is yet additionally provided, in accordance with an embodiment of the present invention, a method for treating a subject, including:

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current to inhibit propagation of naturally-generated efferent action potentials traveling through the site, while inhibiting no more than about 10% of naturally-generated afferent action potentials traveling through the site, so as to treat a condition of the subject.

In an embodiment, the condition includes atrial fibrillation (AF), and configuring the current includes configuring the current so as to treat the AF. In an embodiment, the condition includes bradycardia, and configuring the current includes configuring the current so as to prevent the bradycardia.

In an embodiment, the method includes administering to the subject a drug for treating the condition, and configuring the current includes configuring the current so as to increase an efficacy of the drug.

In an embodiment, configuring the current includes configuring the current to have an amplitude of between about 0.1 and about 15 milliamps. For some applications, configuring the current includes configuring the current to have an amplitude of between about 4 and about 15 milliamps.

In an embodiment, applying the current includes applying the current in respective bursts of pulses in each of a plurality of cardiac cycles of the subject. For some applications, configuring the current includes configuring each of the pulses to have a duration of between about 0.6 and about 2 milliseconds. For some applications, configuring the current includes configuring the pulses within each of the bursts to have a pulse repetition interval of between about 4 and about 20 milliseconds.

There is also provided, in accordance with an embodiment of the present invention, a method including:

selecting a subject who has not been diagnosed with any heart condition;

applying, for a period having a duration of at least about one month, a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to not reduce a heart rate of the subject below a normal heart rate for a typical human.

In an embodiment, the method includes sensing a heart rate of the subject, and configuring the current includes configuring the current so as to reduce the heart rate towards the normal rate, responsive to a determination that the heart rate is greater than the normal rate.

In an embodiment, the method includes sensing a heart rate of the subject, and configuring the current includes configuring the current so as to minimize an effect of applying the current on the heart rate, responsive to a determination that the heart rate is within a desired range.

In an embodiment, the method includes sensing a physiological parameter of the subject, and configuring the current includes configuring the current so as to reduce the heart rate towards the normal rate, responsive to the physiological parameter.

There is further provided, in accordance with an embodiment of the present invention, a method for treating a subject suffering from a condition, including:

applying a current to a site of the subject selected from the list consisting of: a vagus nerve of the subject, and epicardial fat pad of the subject, a pulmonary vein of the subject, a carotid artery of the subject, a carotid sinus of the subject, a vena cava vein of the subject, and an internal jugular vein of the subject; and

configuring the current so as to delay electrical remodeling of an atrium of the subject caused by the condition.

In an embodiment, configuring the current includes configuring the current so as to prevent electrical remodeling of the atrium caused by the condition.

In an embodiment, the condition includes heart failure (HF), and configuring the current includes configuring the current so as to prevent the electrical remodeling caused by the HF.

In an embodiment, the condition includes both atrial fibrillation (AF) and heart failure (HF), and configuring the current includes configuring the current so as to prevent the electrical remodeling caused by the AF and the HF.

In an embodiment, the method includes administering a drug for treating the condition.

In an embodiment, no drug is administered for treating the condition during a period beginning about 24 hours before initiation of application of the current and ending upon the initiation of the application of the current.

In an embodiment, the condition includes atrial fibrillation (AF), and configuring the current includes configuring the current so as to prevent the electrical remodeling caused by the AF. For some applications, applying the current includes detecting an occurrence of the AF, and applying the current responsively to the detecting. For some applications, applying the current includes applying the current not responsively to detecting an occurrence of the AF.

There is still further provided, in accordance with an embodiment of the present invention, a method for treating a subject susceptible to bradycardia, including:

administering to the subject a beta-blocker at a dosage lower than would normally be indicated for the subject;

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

sensing a heart rate of the subject; and

upon detecting an occurrence of the bradycardia, terminating applying the current at least until a cessation of the bradycardia.

In an embodiment, applying the current includes applying the current in respective bursts of pulses in each of a plurality of cardiac cycles of the subject. For some applications, applying the current includes configuring each of the pulses to have a duration of between about 100 microseconds and about 1 millisecond. For some applications, applying the current includes configuring each of the bursts to have a duration of between about 1 and about 60 milliseconds. For some applications, applying the current includes configuring each of the bursts to contain between about 1 and about 5 pulses. For some applications, applying the current includes configuring the pulses within each of the bursts to have a pulse repetition interval of between about 1 and about 10 milliseconds. For some applications, applying the current includes configuring the pulses to have an amplitude of between about 0.1 and about 4 milliamps.

For some applications, applying the current includes applying the bursts once every second heartbeat. For some applications, applying the current includes applying the current to the site intermittently during alternating “on” and “off” periods, each of the “on” periods having a duration of at least about 500 milliseconds. For some applications, applying the current includes applying each of the bursts after a delay following an R-wave of the subject, the delay having a duration of between about 100 and about 700 milliseconds.

There is additionally provided, in accordance with an embodiment of the present invention, a method including:

applying a current to a site of a subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

applying a pacing signal to a heart of the subject; and

configuring the pacing signal to substantially prevent any heart-rate-lowering effects of applying the current.

In an embodiment, applying the current includes applying the current to the site intermittently during alternating “on” and “off” periods, and configuring the pacing signal includes configuring the pacing signal to pace the heart at a rate that is approximately a rate of the heart during the “off” periods.

In an embodiment, applying the pacing signal includes sensing a post-stimulation-initiation heart rate of the subject after initiating application of the current, and applying the pacing signal when the post-stimulation-initiation heart rate is less than a threshold heart rate. For some applications, the method includes sensing a pre-stimulation-initiation heart rate of the subject prior to initiating application of the current, and setting the threshold heart rate equal to the pre-stimulation-initiation heart rate.

In an embodiment, applying the pacing signal includes continuing to apply the pacing signal during a period following termination of applying the current. For some applications, the period has a duration of less than about 30 seconds, and continuing to apply the pacing signal includes continuing to apply the pacing signal during the period having the duration of less than about 30 seconds.

There is yet additionally provided, in accordance with an embodiment of the present invention, a method including:

applying a current to a site of a subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current to reduce mechanical tension on at least one atrium of the subject, so as to reduce a risk of an occurrence of atrial fibrillation (AF).

In an embodiment, the method includes administering to the subject a drug for treating the AF.

There is also provided, in accordance with an embodiment of the present invention, a method for treating a subject suffering from an emergency condition, including:

administering atropine to the subject so as to treat the emergency condition;

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to reduce an adverse effect sometimes caused by the atropine.

There is further provided, in accordance with an embodiment of the present invention, a method for treating a subject, including:

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to treat a condition of the subject selected from the list consisting of: an autoimmune disease, an autoimmune inflammatory disease, multiple sclerosis, encephalitis, myelitis, immune-mediated neuropathy, myositis, dermatomyositis, polymyositis, inclusion body myositis, inflammatory demyelinating polyradiculoneuropathy, Guillain Barre syndrome, myasthenia gravis, inflammation of the nervous system, inflammatory bowel disease, Crohn's disease, ulcerative colitis, SLE (systemic lupus erythematosus), rheumatoid arthritis, vasculitis, polyarteritis nodosa, Sjogren syndrome, mixed connective tissue disease, glomerulonephritis, thyroid autoimmune disease, sepsis, meningitis, a bacterial infection, a viral infection, a fungal infection, sarcoidosis, hepatitis, and portal vein hypertension.

In an embodiment, the control unit is adapted to monitor a heart rate of the subject, and withhold the applying of the current in response to the heart rate being lower than a threshold heart rate.

There is still further provided, in accordance with an embodiment of the present invention, a method for treating a subject, including:

applying a current to a site of the subject selected from the list consisting of: a vagus nerve of the subject, and epicardial fat pad of the subject, a pulmonary vein of the subject, a carotid artery of the subject, a carotid sinus of the subject, a vena cava vein of the subject, and an internal jugular vein of the subject; and

configuring the current so as to have an antiarrhythmic effect on an atrium of the subject.

For some applications, the site includes a right vagus nerve of the subject, and applying the current includes applying the current to the right vagus nerve.

In an embodiment, the method includes administering an antiarrhythmic drug to the subject in conjunction with applying the current.

For some applications, configuring the current includes configuring the current so as to induce rhythmic vagal activity in the subject.

There is additionally provided, in accordance with an embodiment of the present invention, a method for treating a subject suffering from heart failure (HF), including:

applying a current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current so as to decrease atrial contractile force of a heart of the subject, so as to treat the HF.

In an embodiment, applying the current includes applying the current to the site intermittently during alternating “on” and “off” periods. For some applications, applying the current intermittently includes setting each of the “on” periods to have a duration of between about 1 and about 15 seconds, and each of the “off” periods to have a duration of between about 5 and about 20 seconds.

In an embodiment, the site includes the vagus nerve, and applying the current includes applying the current to the vagus nerve. For some applications, applying the current includes applying a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set. For some applications, applying the current includes applying a stimulating current, which is capable of inducing action potentials in the vagus nerve, and an inhibiting current, which is capable of inhibiting action potentials induced by the stimulating current and traveling in the vagus nerve in an afferent direction toward a brain of the subject.

In an embodiment, applying the current includes applying the current in respective bursts of pulses in each of a plurality of cardiac cycles of the subject. For some applications, applying the current includes applying a first pulse of each of the bursts after a delay from a sensed feature of an electrocardiogram (ECG) of the subject.

In an embodiment, the method includes sensing a physiological parameter of the subject, and configuring the current includes configuring the current at least in part responsively to the sensed physiological parameter. For some applications, sensing the physiological parameter includes sensing a heart rate of the subject.

In an embodiment, configuring the current includes configuring the current so as to minimize an effect of the applying of the current on a heart rate of the subject.

In an embodiment, applying the current includes applying the current in respective bursts of pulses in each of a plurality of cardiac cycles of the subject. For some applications, applying the current includes applying the current to a left vagus nerve of the subject. For some applications, applying the current includes configuring each of the pulses to have a duration of between about 200 microseconds and about 2.5 milliseconds. For some applications, applying the current includes configuring each of the pulses to have a duration of between about 2.5 and about 5 milliseconds. For some applications, applying the current includes configuring each of the bursts to have a duration of between about 0.2 and about 40 milliseconds. For some applications, applying the current includes configuring each of the bursts to contain between about 1 and about 10 pulses. For some applications, applying the current includes configuring the pulses within each of the bursts to have a pulse repetition interval of between about 2 and about 10 milliseconds. For some applications, applying the current includes configuring the pulses to have an amplitude of between about 0.5 and about 5 milliamps. For some applications, applying the current includes applying the bursts less than every heartbeat of the subject. For some applications, applying the current includes applying the bursts once per heartbeat of the subject. For some applications, applying the current includes applying the current to the site intermittently during alternating “on” and “off” periods, each of the “on” periods having a duration of at least about 1 second. For some applications, applying the current includes applying each of the bursts after a variable delay following a P-wave of the subject, the delay having a duration equal to between about two-thirds and about 90% of a duration of a cardiac cycle of the subject. For some applications, applying the current includes substantially continuously measuring the duration of the cardiac cycle.

In an embodiment, applying the current includes applying the current in respective bursts of pulses in each of a plurality of cardiac cycles of the subject. For some applications, applying the current includes configuring each of the pulses to have a duration of between about 100 microseconds and about 2.5 milliseconds. For some applications, applying the current includes configuring each of the bursts to have a duration of between about 1 and about 180 milliseconds. For some applications, applying the current includes configuring each of the bursts to contain between about 1 and about 10 pulses. For some applications, applying the current includes configuring the pulses within each of the bursts to have a pulse repetition interval of between about 1 and about 20 milliseconds. For some applications, applying the current includes configuring the pulses to have an amplitude of between about 0.1 and about 9 milliamps. For some applications, applying the current includes applying the bursts once every second heartbeat. For some applications, applying the current includes applying the bursts once every third heartbeat. For some applications, applying the current includes applying the current to the site intermittently during alternating “on” and “off” periods, each of the “on” periods having a duration of at least about 1 second. For some applications, applying the current includes applying each of the bursts after a delay following an R-wave of the subject, the delay having a duration of about 100 milliseconds.

In an embodiment, applying the current includes applying the current in respective bursts of between about 1 and about 10 pulses in each of a plurality of cardiac cycles of the subject, and applying a first pulse of each of the bursts after a delay of about 100 milliseconds after a sensed R-wave of an electrocardiogram (ECG) of the subject. For some applications, applying the current includes configuring each of the bursts to contain about three pulses. For some applications, applying the current includes varying a number of the pulses in each of the bursts responsive to a sensed parameter of a respiratory cycle of the subject. For some applications, applying the current includes varying a number of the pulses in each of the bursts responsive to a sensed heart rate of the subject. For some applications, the site includes the vagus nerve, and applying the current includes applying the current to the vagus nerve, and, responsive to a sensed heart rate of the subject, varying a number of nerve fibers of the vagus nerve that are recruited.

For some applications, the site includes the vagus nerve, and applying the current includes applying the current to the vagus nerve, and, responsive to a sensed parameter of a respiratory cycle of the subject, varying a number of nerve fibers of the vagus nerve that are recruited. For some applications, applying the current includes cycling between a first set of parameters and a second set of parameters. For some applications, cycling includes applying each set of parameters for less than about 15 seconds. For some applications, cycling includes applying each set of parameters for between about 1 and about 4 seconds. For some applications, the first set of parameters includes a first amplitude, the second set of parameters includes a second amplitude, greater than the first amplitude, and applying the current includes varying a number of nerve fibers of the vagus nerve that are recruited by cycling between the first set of parameters and the second set of parameters.

For some applications, cycling includes synchronizing application of the first set of parameters with inhalation by the subject, and synchronizing application of the second set of parameters with exhalation by the subject. For some applications, at least one of the first and second sets of parameters includes a pulse repetition interval of between about 4 and about 20 milliseconds, and applying the current includes cycling between the first and second sets of parameters. For some applications, at least one of the first and second sets of parameters includes a pulse width of between about 0.1 and about 2 milliseconds, and applying the current includes cycling between the first and second sets of parameters. For some applications, the first set of parameters includes application of the current at one pulse per each of the bursts, the second set of parameters includes application of the current at about three pulses per each of the bursts, and applying the current includes cycling between the first and second sets of parameters.

There is yet additionally provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to enhance an efficacy of a drug administered to the subject for treating a condition from which the subject suffers selected from the list consisting of: atrial fibrillation (AF) and heart failure (HF).

There is also provided, in accordance with an embodiment of the present invention, a system for treating a subject, including:

a drug, adapted to be administered to the subject, and to treat a condition from which the subject suffers selected from the list consisting of: atrial fibrillation (AF) and heart failure (HF); and

apparatus including:
an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and
a control unit, adapted to:
drive the electrode device to apply an electrical current to the site, and
configure the current so as to enhance an efficacy of the drug.

There is further provided, in accordance with an embodiment of the present invention, apparatus for treating a subject suffering from a condition, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current to increase vagal tone of the subject, and to minimize an effect of applying the current on a heart rate of the subject, so as to treat the condition.

There is still further provided, in accordance with an embodiment of the present invention, apparatus for treating a subject suffering from a condition, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to reduce an adverse effect sometimes caused by a drug administered to the subject for treating the condition.

There is additionally provided, in accordance with an embodiment of the present invention, a system for treating a subject suffering from a condition, including:

a drug, adapted to be administered to the subject, and to treat the condition; and apparatus including:
an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and
a control unit, adapted to:
drive the electrode device to apply an electrical current to the site, and
configure the current so as to reduce an adverse effect sometimes caused by the drug.

There is yet additionally provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to reduce a heart condition of the subject selected from the list consisting of: fibrosis of the heart, and inflammation of the heart.

For some applications, in an operating mode of the control unit, the control unit is adapted to drive the electrode device to apply the current during an application period lasting at least about three weeks, and to configure the current such that, during the application period, a longest duration of time in which no current is applied is less than four hours.

There is also provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current to inhibit propagation of naturally-generated efferent action potentials traveling through the site, while inhibiting no more than about 10% of naturally-generated afferent action potentials traveling through the site, so as to treat a condition of the subject.

There is further provided, in accordance with an embodiment of the present invention, apparatus for treating a subject who has not been diagnosed with any heart condition, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site for a period having a duration of at least about one month, and

configure the current so as to not reduce a heart rate of the subject below a normal heart rate for a typical human.

There is still further provided, in accordance with an embodiment of the present invention, apparatus for treating a subject suffering from a condition, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to delay electrical remodeling of an atrium of the subject caused by the condition.

There is additionally provided, in accordance with an embodiment of the present invention, apparatus including:

a pacemaker, adapted to be coupled to a heart of a subject;

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site,

drive the pacemaker to apply a pacing signal to the heart, and

configure the pacing signal to substantially prevent any heart-rate-lowering effects of applying the current.

There is also provided, in accordance with an embodiment of the present invention, apparatus including:

an electrode device, adapted to be coupled to a site of a subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current to reduce mechanical tension on at least one atrium of the subject, so as to reduce a risk of an occurrence of atrial fibrillation (AF).

There is further provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to treat a condition of the subject selected from the list consisting of: an autoimmune disease, an autoimmune inflammatory disease, multiple sclerosis, encephalitis, myelitis, immune-mediated neuropathy, myositis, dermatomyositis, polymyositis, inclusion body myositis, inflammatory demyelinating polyradiculoneuropathy, Guillain Barre syndrome, myasthenia gravis, inflammation of the nervous system, inflammatory bowel disease, Crohn's disease, ulcerative colitis, SLE (systemic lupus erythematosus), rheumatoid arthritis, vasculitis, polyarteritis nodosa, Sjogren syndrome, mixed connective tissue disease, glomerulonephritis, thyroid autoimmune disease, sepsis, meningitis, a bacterial infection, a viral infection, a fungal infection, sarcoidosis, hepatitis, and portal vein hypertension.

There is still further provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to have an antiarrhythmic effect on an atrium of the subject.

There is additionally provided, in accordance with an embodiment of the present invention, apparatus for treating a subject suffering from heart failure (HF), including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current so as to decrease atrial contractile force of a heart of the subject, so as to treat the HF.

There is also provided, in accordance with an embodiment of the present invention, apparatus for treating a subject suffering from spontaneous atrial fibrillation (AF), including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current to maintain the spontaneous AF for at least about 24 hours, so as to treat the subject.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the site includes the vagus nerve, the electrode device is adapted to be coupled to the vagus nerve, the current includes a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve.

For some applications, the control unit is adapted to drive the electrode device to configure the current to maintain the AF for between about 24 hours and about three weeks. Alternatively, the control unit is adapted to drive the electrode device to configure the current to maintain the AF for at least about three weeks.

In an embodiment, the apparatus includes a sensor adapted to detect normal sinus rhythm (NSR) and generate a sensor signal responsive thereto, and the control unit is adapted to receive the sensor signal, and to drive the electrode device to apply the current responsive to the sensor signal.

In an embodiment, the apparatus includes a sensor adapted to detect the AF and generate a sensor signal responsive thereto, and the control unit is adapted to receive the sensor signal, and to drive the electrode device to apply the current responsive to the sensor signal.

In an embodiment, the apparatus including a cardiac electrode device, adapted to be coupled to cardiac tissue of the subject, and the control unit is adapted to:

drive the cardiac electrode device to apply a cardiac electrical current to the cardiac tissue, and

configure the cardiac electrical current to maintain the spontaneous AF, so as to treat the subject.

For some applications, the control unit is adapted to drive the electrode device to apply the current with an amplitude of between about 2 and about 5 milliamps.

In an embodiment, the control unit is adapted to drive the electrode device to apply the current in respective bursts in each of a plurality of cardiac cycles of the subject. For some applications, the control unit is adapted to configure each pulse of each of the bursts to have a pulse duration of between about 1 and about 3 milliseconds. For some applications, the control unit is adapted to configure each burst to have between about 1 and about 8 pulses. For some applications, the control unit is adapted to configure each pulse of each of the bursts to have a pulse duration of between about 0.5 and about 3 milliseconds. For some applications, the control unit is adapted to configure each of the bursts to contain between about 1 and about 100 pulses.

For some applications, the apparatus includes a sensor adapted to detect a complex in a cardiac rhythm of the subject, and generate a sensor signal responsive thereto, and the control unit is adapted to receive the sensor signal, and to drive the electrode device to apply the current responsive to the sensor signal.

There is also provided, in accordance with an embodiment of the present invention, apparatus for treating a subject suffering from spontaneous atrial fibrillation (AF), including:

an electrode device, adapted to be coupled to tissue of the subject; and

a control unit, adapted to:

drive the electrode device to apply an electrical current to the tissue, and

configure the current to maintain the spontaneous AF for at least about 24 hours, so as to treat the subject.

For some applications, the control unit is adapted to configure the current to maintain the AF for between about 24 hours and about three weeks. Alternatively, the control unit is adapted to configure the current to maintain the AF for at least about three weeks.

In an embodiment, the apparatus includes a sensor adapted to detect normal sinus rhythm (NSR) and generate a sensor signal responsive thereto, and the control unit is adapted to receive the sensor signal, and to drive the electrode device to apply the current responsive to the sensor signal.

In an embodiment, the apparatus includes a sensor adapted to detect the AF and generate a sensor signal responsive thereto, and the control unit is adapted to receive the sensor signal, and to drive the electrode device to apply the current responsive to the sensor signal.

For some applications, the control unit is adapted to drive the electrode device to apply the current at a frequency of at least about 3 Hz.

In an embodiment, the tissue includes cardiac tissue of the subject, and the electrode device is adapted to be coupled to the cardiac tissue. In an embodiment, the tissue is selected from the list consisting of: atrial tissue, cardiac fat pad tissue, a pulmonary vein, a carotid artery, a carotid sinus, a vena cava vein, and an internal jugular vein, and the electrode device is adapted to be coupled to the selected tissue.

There is further provided, in accordance with an embodiment of the present invention, treatment apparatus, including:

an electrode device, adapted to be coupled to tissue of a subject; and

a control unit, adapted to:

drive the electrode device to apply an electrical current to the tissue, and

configure the current to modify atrial motion of the subject to a level sufficient to reduce a risk of an occurrence of a thromboembolic event.

In an embodiment, the control unit is adapted to configure the current to modify blood flow within an atrium of the subject.

In an embodiment, the electrode device is adapted to be coupled to the tissue of the subject, the subject suffering from atrial fibrillation (AF) or from increased risk of thromboembolic events.

In an embodiment, the control unit is adapted to configure the current to increase blood flow out of a left atrial auricle of the subject.

In an embodiment, the apparatus includes a sensor adapted to detect an occurrence of atrial fibrillation (AF) and generate a sensor signal responsive thereto, and the control unit is adapted to receive the sensor signal, and to drive the electrode device to apply the current during the occurrence of the AF.

In an embodiment, the apparatus includes a sensor adapted to detect an occurrence of atrial fibrillation (AF) and generate a sensor signal responsive thereto, and the control unit is adapted to drive the electrode device to apply the current in the absence of the occurrence of the AF.

In an embodiment, the tissue includes cardiac tissue of the subject, and the electrode device is adapted to be coupled to the cardiac tissue. In an embodiment, the tissue is selected from the list consisting of: atrial tissue, cardiac fat pad tissue, a pulmonary vein, a carotid artery, a carotid sinus, a vena cava vein, and an internal jugular vein, and the electrode device is adapted to be coupled to the selected tissue.

In an embodiment, the tissue includes a vagus nerve of the subject, and the electrode device is adapted to be coupled to the vagus nerve. In an embodiment, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve.

In an embodiment, the control unit is adapted to:

during a first stimulation period, configure the current to cause a reduction in a force of contraction of atrial cells of the subject, and

during a second stimulation period, configure the current to cause an increase in the reduced force of contraction of the atrial cells.

For some applications, the control unit is adapted to set the first stimulation period to have a duration of between about 100 milliseconds and about 1000 milliseconds. Alternatively, the control unit is adapted to set the second stimulation period to have a duration of between about 200 milliseconds and about 15 seconds. For some applications, the control unit is adapted to configure the current to have a first frequency during the first stimulation period, and a second frequency during the second stimulation period, the first frequency greater than the second frequency.

For some applications, the control unit is adapted to configure the current to have a first amplitude during the first stimulation period, and a second amplitude during the second stimulation period, the first amplitude greater than the second amplitude.

In an embodiment, the control unit is adapted to:

drive the electrode device to apply the current during the first stimulation period, and

withhold the electrode device from applying the current during the second stimulation period.

In an embodiment, the control unit is adapted to:

during the first stimulation period, configure the current so as to induce action potentials in the vagus nerve, and

during the second stimulation period, configure the current so as to block action potentials in the vagus nerve.

In an embodiment, the control unit is adapted to configure the current so as to induce action potentials in the vagus nerve during the first and the second stimulation periods.

In an embodiment, the control unit is adapted to:

drive the electrode device to apply the current in respective bursts in each of a plurality of cardiac cycles of the subject, and

configure each pulse of each of the bursts to have a pulse width of at least a first pulse width during the first stimulation period, and to have a pulse width of less than a second pulse width during the second stimulation period, the first pulse width being greater than or equal to the second pulse width.

In an embodiment, the control unit is adapted to:

drive the electrode device to apply the current in respective bursts in each of a plurality of cardiac cycles of the subject, and

configure each of the bursts to have a number of pulses of at least a first number of pulses during the first stimulation period, and to have a number of pulses of less than a second number of pulses during the second stimulation period, the first number of pulses being greater than or equal to the second number of pulses.

In an embodiment, the apparatus includes a sensor, adapted to sense at least one physiological variable of the subject, and to generate a sensor signal responsive thereto, and the control unit is adapted to receive the sensor signal and to synchronize therewith a commencement of at least one of the first and second stimulation periods. For some applications, the sensed physiological variable includes a QRS-complex of the subject, and the control unit is adapted to initiate the first stimulation period within about 50 milliseconds after an occurrence of the QRS-complex. Alternatively or additionally, the sensed physiological variable includes an expiration by the subject, and the control unit is adapted to initiate the first stimulation period within about 500 milliseconds after a beginning of the expiration. Further alternatively or additionally, the sensed physiological variable includes diastole of the subject, and the control unit is adapted to initiate the second stimulation period substantially simultaneously with a portion of the diastole.

There is still further provided, in accordance with an embodiment of the present invention, treatment apparatus, including:

an electrode device, adapted to be coupled to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

repeatedly change at least one parameter of the current, so as to restore normal sinus rhythm (NSR) of the subject.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve. For some applications, the parameter includes an amplitude of the current, and the control unit is adapted to repeatedly change the amplitude. Alternatively or additionally, the parameter includes a frequency of the current, and the control unit is adapted to repeatedly change the frequency.

In an embodiment, the control unit is adapted to drive the electrode device to apply the current in respective bursts in each of a plurality of cardiac cycles of the subject, the parameter includes a number of pulses in each of the bursts, and the control unit is adapted to repeatedly change the number of pulses in each of the bursts.

In an embodiment, the control unit is adapted to drive the electrode device to apply the current in respective bursts in each of a plurality of cardiac cycles of the subject, the parameter includes a pulse width of pulses in each of the bursts, and the control unit is adapted to repeatedly change the pulse width of the pulses in each of the bursts.

In an embodiment, the control unit is adapted to drive the electrode device to apply the electrical current in pulses, the parameter includes a pulse width of the pulses, and the control unit is adapted to repeatedly change the pulse width.

In an embodiment, the parameter includes an on/off status of the current, and the control unit is adapted to repeatedly change the on/off status. For some applications, the control unit is adapted to repeatedly change a duration of at least one period selected from the list consisting of: an “on” period of the current, and an “off” period of the current.

In an embodiment, the control unit is adapted to:

during a first period, configure the current so as to induce action potentials in the site, and

during a second period, configure the current so as to block action potentials in the site.

In an embodiment, the control unit is adapted to repeatedly change the parameter at a rate of between about one change per heart beat of the subject and about one change per 30 seconds.

In an embodiment, the control unit is adapted to repeatedly change the parameter according to a predetermined pattern. Alternatively or additionally, the control unit is adapted to repeatedly change the parameter randomly. For some applications, the control unit is adapted to repeatedly change the parameter randomly, with an interval between each change of between about 500 milliseconds and about 30 seconds.

In an embodiment, the apparatus includes a sensor, adapted to detect an occurrence of the AF and generate a sensor signal indicative thereof, and the control unit is adapted to receive the sensor signal, and to drive the electrode device to apply the current responsive to the sensor signal.

There is additionally provided, in accordance with an embodiment of the present invention, treatment apparatus, including:

an electrode device, adapted to be coupled to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

a pacing device, adapted to be applied to a heart of the subject; and

a control unit, adapted to:

during a first period, drive the pacing device to pace the heart, and drive the electrode device to apply an electrical current to the site, and

during a second period following the first period, withhold the electrode device from applying the electrical current to the site.

In an embodiment, the control unit is adapted to configure a parameter of at least one of the periods to be such as to restore normal sinus rhythm (NSR) of the subject within 2 hours after initiation of the second period.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the site includes the vagus nerve, the electrode device is adapted to be coupled to the vagus nerve, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve.

In an embodiment, the control unit is adapted to withhold the pacing device from pacing the heart during at least a portion of the second period.

In an embodiment, the control unit is adapted to configure the first period to have a duration of between about 500 milliseconds and about 30 seconds.

In an embodiment, the control unit is adapted to drive the electrode device to apply the electrical current substantially without changing the parameter during the first period, and with an amplitude greater than about 6 milliamps.

In an embodiment, the apparatus includes a sensor, adapted to detect an occurrence of the AF and generate a sensor signal indicative thereof, and the control unit is adapted to receive the sensor signal, and to drive the pacing device and drive the electrode device to apply the electrical current responsive to the sensor signal.

In an embodiment, the apparatus includes a sensor, adapted to detect an occurrence of the AF and generate a sensor signal indicative thereof, and the control unit is adapted to receive the sensor signal, and to withhold the electrode device from applying the electrical current responsive to the sensor signal.

In an embodiment, the control unit is adapted to configure the pacing device to pace the heart by applying a pacing signal to the heart having a pulse repetition interval having a duration of between about 50% and about 200% of an atrial refractory period of the subject.

In an embodiment, the control unit is adapted to configure the current to modulate an atrial refractory period of the subject.

In an embodiment, the control unit is adapted to configure a parameter of the current selected from the list consisting of: an on/off time of the current, an amplitude of the current, a number of pulses of the current, a pulse repetition interval of the current, a frequency of pulses within a pulse burst of the current, a pulse width of pulses of the current, pulses per trigger of the current, a duty cycle of the current, and timing of the current within a cardiac cycle of the subject.

In an embodiment, the control unit is adapted to configure a parameter of the pacing selected from the list consisting of: an on/off time of the pacing, an amplitude of the pacing, a number of pulses of the pacing, a pulse repetition interval of the pacing, a frequency of pulses within a pulse burst of the pacing, a pulse width of pulses of the pacing, pulses per trigger of the pacing, a duty cycle of the pacing, and timing of the pacing within a cardiac cycle of the subject.

There is yet additionally provided, in accordance with an embodiment of the present invention, treatment apparatus, including:

an electrode device, adapted to be coupled to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

a pacing device, adapted to be applied to a heart of the subject;

a sensor, adapted to detect an occurrence of the AF and generate a sensor signal indicative thereof; and

a control unit, adapted to:

during a first period, drive the pacing device to pace the heart, and drive the electrode device to apply an electrical current to the site, and

responsive to the sensor signal, during a second period following the first period, withhold the electrode device from applying the electrical current to the site.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the site includes the vagus nerve, the electrode device is adapted to be coupled to the vagus nerve, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve.

In an embodiment, the control unit is adapted to, during the first period, drive the pacing device and drive the electrode device to apply the current responsive to the sensor signal.

In an embodiment, the control unit is adapted to withhold the pacing device from pacing the heart during at least a portion of the second period.

In an embodiment, the control unit is adapted to drive the electrode device to apply the electrical current substantially without changing a parameter of the current during the first period, and with an amplitude greater than about 6 milliamps.

In an embodiment, the control unit is adapted to withhold the electrode device from applying the electrical current during the second period responsive to an indication in the sensor signal of a P-wave of the subject.

In an embodiment, the sensor is adapted to generate the sensor signal responsive to a measure of at least one ventricular response parameter, the parameter selected from the list consisting of: a ventricular response rate and a ventricular response variability.

In an embodiment, the sensor is adapted to generate the sensor signal responsive to a measure of pressure, selected from the list consisting of: atrial pressure, venous pressure, and arterial pressure.

In an embodiment, the sensor signal includes a first sensor signal and a second sensor signal, the first sensor signal includes a measure of pressure, selected from the list consisting of: atrial pressure, venous pressure, and arterial pressure, the second sensor signal includes an indication of ventricular contraction, the sensor is adapted to generate the first and the second sensor signals, and the control unit is adapted to receive the first and the second sensor signals, and to detect the AF by analyzing at least one relationship between the first and the second sensor signals.

In an embodiment, the sensor signal includes an electrocardiogram (ECG) signal, the sensor is adapted to measure the ECG signal, and the control unit is adapted to receive the ECG signal, and to detect the AF by analyzing a duration of an isoelectrical segment of the ECG signal.

There is also provided, in accordance with an embodiment of the present invention, treatment apparatus, including:

an electrode device, adapted to be coupled to a site of a subject suffering from atrial fibrillation (AF) principally caused by heightened adrenergic tone, the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to drive the electrode device to apply to the site an electrical stimulating current, which current is capable of inducing action potentials in the site, the current configured to be such as to restore normal sinus rhythm (NSR) of the subject.

In an embodiment, the control unit is adapted to drive the electrode device to apply the current, substantially without changing a parameter of the current.

In an embodiment, the site includes the vagus nerve, the electrode device is adapted to be coupled to the vagus nerve, the control unit is adapted to configure the stimulating current so as to induce action potentials in a first set and a second set of nerve fibers of the vagus nerve, and the control unit is adapted to drive the electrode device to apply to the vagus nerve an inhibiting current, which is capable of inhibiting the induced action potentials traveling in the second set of nerve fibers, the nerve fibers in the second set having generally larger diameters than the nerve fibers in the first set.

In an embodiment, the site includes the vagus nerve, the electrode device is adapted to be coupled to the vagus nerve, the control unit is adapted to drive the electrode device to apply to the vagus nerve an inhibiting current, which is capable of inhibiting device-induced action potentials traveling in the vagus nerve in an afferent direction towards a brain of the subject.

In an embodiment, the apparatus includes a sensor, adapted to detect an occurrence of the AF and generate a sensor signal indicative thereof, and the control unit is adapted to receive the sensor signal, and to drive the electrode device to apply the stimulating current responsive to the sensor signal.

In an embodiment, the control unit is adapted to apply the stimulating current in respective bursts in each of a plurality of cardiac cycles of the subject, each pulse of each of the bursts having a pulse width of between about 0.5 milliseconds and about 1.5 milliseconds.

In an embodiment, the control unit is adapted to apply the stimulating current in respective bursts in each of a plurality of cardiac cycles of the subject, each of the bursts having between about 1 and about 10 pulses.

In an embodiment, the control unit is adapted to apply the stimulating current in respective bursts synchronized with a cardiac cycle of the subject. for some applications, the control unit is adapted to apply a first pulse of each of the bursts after a delay from a sensed feature of an electrocardiogram (ECG) of the subject. For some applications, the sensed feature is selected from the list consisting of: a P-wave of the ECG and an R-wave of the ECG, and the control unit is adapted to apply the first pulse after the delay from the selected sensed feature.

There is further provided, in accordance with an embodiment of the present invention, apparatus for use during defibrillation of a subject suffering from atrial fibrillation (AF), including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply an electrical current to the site, and

configure the current to cause bradycardia and a decreased level of alertness during the defibrillation.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve.

In an embodiment, the site includes the vagus nerve, the electrode device is adapted to be coupled to the vagus nerve, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve.

76048

In an embodiment, the site includes the vagus nerve, the electrode device is adapted to be coupled to the vagus nerve, and the control unit is adapted to:

apply an inhibiting electrical signal to the vagus nerve, and

configure the inhibiting signal to block action potentials traveling in the vagus nerve in an afferent direction toward a brain of the subject.

In an embodiment, the apparatus includes a pacing device, adapted to be applied to a heart of the subject, and the control unit is adapted to drive the pacing device to pace the heart if a heart rate of the subject falls below a predetermined rate responsive to application of the current configured to cause the decreased level of alertness.

In an embodiment, the control unit is adapted to drive the electrode device to apply the current with an amplitude of between about 4 and about 8 milliamps.

In an embodiment, the control unit is adapted to drive the electrode device to apply the current in respective bursts in each of a plurality of cardiac cycles of the subject. For some applications, the control unit is adapted to configure each pulse of each of the bursts to have a pulse duration of between about 1 and about 3 milliseconds. For some applications, the control unit is adapted to configure each burst to have between about 6 and about 10 pulses.

There is still further provided, in accordance with an embodiment of the present invention, apparatus for treating a subject suffering from atrial fibrillation (AF), including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

a sensor, adapted to be applied to tissue of the subject, and to generate at least one sensor signal responsive to a sensed physiological parameter of the subject; and

a control unit, adapted to:

detect the AF by receiving and analyzing the at least one sensor signal,

responsive to detecting the AF, drive the electrode device to apply an electrical current to the site,

during a first period beginning upon detecting the AF, configure the current to attempt to restore normal sinus rhythm (NSR) of the subject,

determine whether NSR has been restored, and

during a second period beginning responsive to determining that NSR has not been restored within a threshold period of time after detecting the AF, configure the current to maintain AF.

In an embodiment, the site includes the vagus nerve, the electrode device is adapted to be coupled to the vagus nerve, the control unit is adapted to configure the current to include a stimulating current, which is capable of inducing action potentials in a first set and a second set of nerve fibers of the vagus nerve, and an inhibiting current, which is capable of inhibiting the induced action potentials traveling in the second set of nerve fibers, the nerve fibers in the second set having generally larger diameters than the nerve fibers in the first set, and the control unit is adapted to drive the electrode device to apply the stimulating current and the inhibiting current to the vagus nerve, responsive to detecting the AF.

In an embodiment, the site includes the vagus nerve, the electrode device is adapted to be coupled to the vagus nerve, the control unit is adapted to configure the current to include a stimulating current, which is capable of inducing action potentials in the vagus nerve, and an inhibiting current, which is capable of inhibiting device-induced action potentials traveling in the vagus nerve in an afferent direction toward a brain of the subject, and the control unit is adapted to drive the electrode device to apply the stimulating current and the inhibiting current to the vagus nerve, responsive to detecting the AF.

In an embodiment, the sensed physiological parameter includes a P-wave of the subject, and the sensor is adapted to generate the sensor signal responsive to the P-wave. Alternatively or additionally, the sensed physiological parameter includes a measure of at least one ventricular response parameter of the subject, the parameter selected from the list consisting of: a ventricular response rate and a ventricular response variability, and the sensor is adapted to generate the sensor signal responsive to the ventricular response parameter. Further alternatively or additionally, the sensed physiological parameter includes a measure of pressure of the subject, selected from the list consisting of: atrial pressure, venous pressure, and arterial pressure, and the sensor is adapted to generate the sensor signal responsive to the measure of the pressure.

In an embodiment, the sensed physiological parameter includes a first sensed physiological parameter and a second sensed physiological parameter, the first sensed physiological parameter includes a measure of pressure of the subject, selected from the list consisting of: atrial pressure, venous pressure, and arterial pressure, the second sensed physiological parameter includes an indication of ventricular contraction of the subject, the sensor is adapted to generate a first sensor signal and a second sensor signal responsive to the measure of pressure and the indication of ventricular contraction, respectively, and the control unit is adapted to receive the first and the second sensor signals, and to detect the AF by analyzing at least one relationship between the first and the second sensor signals.

In an embodiment, the sensed physiological parameter includes an electrocardiogram (ECG) signal of the subject, the sensor is adapted to generate the sensor signal responsive to the ECG signal, and the control unit is adapted to receive the sensor signal, and to detect the AF by analyzing a duration of an isoelectrical segment of the ECG signal.

In an embodiment, the control unit is adapted to configure the current to attempt to restore NSR by repeatedly changing at least one parameter of the current.

In an embodiment, the apparatus includes a pacing device, adapted to be applied to a heart of the subject, and the control unit is adapted to attempt to restore NSR during the first period by:

during a pacing period within the first period, driving the pacing device to pace the heart, and driving the electrode device to apply the current to the site, and

during a withholding period following the pacing period, withholding the electrode device from applying the current to the site.

For some applications, the control unit is adapted to configure the pacing device to pace the heart by applying a pacing signal to the heart having a pulse repetition interval having a duration of between about 50% and about 200% of an atrial refractory period of the subject.

For some applications, the control unit is adapted to configure the current to modulate an atrial refractory period of the subject.

For some applications, the control unit is adapted to configure a parameter of the current selected from the list consisting of: an on/off time of the current, an amplitude of the current, a number of pulses of the current, a pulse repetition interval of the current, a frequency of pulses within a pulse burst of the current, a pulse width of pulses of the current, pulses per trigger of the current, a duty cycle of the current, and timing of the current within a cardiac cycle of the subject.

For some applications, the control unit is adapted to configure a parameter of the pacing selected from the list consisting of: an on/off time of the pacing, an amplitude of the pacing, a number of pulses of the pacing, a pulse repetition interval of the pacing, a frequency of pulses within a pulse burst of the pacing, a pulse width of pulses of the pacing, pulses per trigger of the pacing, a duty cycle of the pacing, and timing of the pacing within a cardiac cycle of the subject.

In an embodiment, the control unit is adapted to generate a notification signal upon determining that NSR has been restored.

In an embodiment, the control unit is adapted to maintain a duration of the threshold period between about 24 and 54 hours. For some applications, the control unit is adapted to maintain a duration of the threshold period between about 44 and 52 hours.

In an embodiment, the control unit is adapted to record a time of detecting of the AF. For some applications, the control unit is adapted to output the recorded time upon interrogation by a user.

There is additionally provided, in accordance with an embodiment of the present invention, apparatus for nerve stimulation, including an electrode device,

adapted to be coupled to a nerve of a subject, the nerve including a first set of fibers situated in a vicinity of an external surface of the nerve, and a second set of fibers situated in a vicinity of a longitudinal axis of the nerve, and

adapted to generate an electrical field defining a first activation function at the first set of fibers, and defining a second activation function at the second set of fibers, the first activation function being less than about four times greater than the second activation function.

In an embodiment, the electrode device is adapted to be fixed to the nerve.

In an embodiment, the electrode device includes one or more electrodes having respective conductive surfaces, which are adapted to be coupled to the nerve such that a distance between each of the conductive surfaces and the axis of the nerve is at least about 0.5 millimeters.

In an embodiment, the nerve includes a vagus nerve of the subject, and the electrode device is adapted to be coupled to the vagus nerve.

In an embodiment, the electrode device is adapted to generate the electrical field by applying a current having an amplitude of at least 5 milliamps. For some applications, the electrode device is adapted to generate the electrical field by applying the current having an amplitude of at least 7 milliamps.

There is yet additionally provided, in accordance with an embodiment of the present invention, apparatus for nerve stimulation, including:

one or more electrodes having respective conductive surfaces, which are adapted to be coupled to a nerve of a subject such that a distance between each of the conductive surfaces and an axis of the nerve is at least about 0.5 millimeters; and

a control unit, adapted to drive the electrodes to apply a current having an amplitude of at least 5 milliamps.

In an embodiment, the apparatus includes one or more insulating elements that separate the electrodes from one another, such that a distance between each of the insulating elements and the axis of the nerve is between about 0.5 and about 3 millimeters.

In an embodiment, the control unit is adapted to drive the electrodes to apply the current having an amplitude of at least 7 milliamps.

In an embodiment, the nerve includes a vagus nerve of the subject, and the electrodes are adapted to be coupled to the vagus nerve.

In an embodiment, the electrodes are adapted to be coupled to the nerve such that the distance between each of the conductive surfaces and the axis of the nerve is at least about 1.5 millimeters.

In an embodiment, the electrodes are adapted to be coupled to the nerve such that the distance between each of the conductive surfaces and the axis of the nerve is less than about 2 millimeters.

In an embodiment, the electrodes are adapted to be coupled to the nerve such that the distance between each of the conductive surfaces and the axis of the nerve is at least about 3 millimeters.

There is also provided, in accordance with an embodiment of the present invention, apparatus for stimulating a nerve of a subject, the nerve including small-, medium-, and large-diameter fibers, the apparatus including:

a cathode, adapted to be disposed at a cathodic site of the nerve, and to apply a cathodic current to the nerve which is capable of inducing action potentials in the nerve;

an anode, adapted to be disposed at an anodal site of the nerve, and to apply to the nerve an anodal current which is capable of inhibiting action potentials in the nerve; and

a control unit, adapted to:

drive the cathode to apply to the nerve the cathodic current having a cathodic amplitude sufficient to induce action potentials in the medium- and large-diameter fibers, but generally insufficient to induce action potentials in the small-diameter fibers, and

simultaneously drive the anode to apply to the nerve the anodal current having an anodal amplitude sufficient to inhibit action potentials in the large-diameter fibers, but generally insufficient to inhibit action potentials in the medium-diameter fibers.

In an embodiment, the nerve includes a vagus nerve of the subject, the cathode is adapted to be disposed at the cathodic site of the vagus nerve, and the anode is adapted to be disposed at the anodal site of the vagus nerve.

In an embodiment, the nerve includes a first set of fibers situated in a vicinity of an external surface of the nerve, and a second set of fibers situated in a vicinity of a longitudinal axis of the nerve, and the cathode is adapted to generate an electrical field defining a first activation function at the first set of fibers, and defining a second activation function at the second set of fibers, the first activation function less than about four times greater than the second activation function.

For some applications, the control unit is adapted to set the cathodic amplitude to be between about 1 and about 10 milliamps. For some applications, according to claim 128, the control unit is adapted to set the anodal amplitude to be between about 1 and about 10 milliamps.

In an embodiment, the apparatus includes a suppression anode, adapted to:

be disposed at a suppression anodal site of the nerve so that the cathodic site is between the anodal site and the suppression anodal site, and

apply to the nerve a suppression anodal current having a suppression anodal amplitude sufficient to inhibit action potentials induced in the nerve by the cathodic current and propagating in a direction from the cathodic site towards the suppression anodal site. For some applications, the suppression anode is adapted to apply the suppression anodal current with the suppression anodal amplitude sufficient to inhibit a portion of the action potentials induced in the nerve by the cathodic current and propagating towards the suppression anodal site.

There is further provided, in accordance with an embodiment of the present invention, apparatus, including:

an electrode device, adapted to be coupled to a nerve of a subject; and

a control unit, adapted to:

drive the electrode device to apply to the nerve a stimulating current, which has a stimulating amplitude sufficient to induce action potentials in a first set and a second set of nerve fibers of the nerve, but not in a third set of nerve fibers of the nerve, the nerve fibers in the first set having generally larger diameters than the nerve fibers in the second set, and the nerve fibers in the second set having generally larger diameters than the nerve fibers in the third set, and

drive the electrode device to apply to the nerve an inhibiting current, which has an inhibiting amplitude sufficient to inhibit the induced action potentials in the first set of nerve fibers, but not in the second set of nerve fibers.

In an embodiment, the nerve includes a vagus nerve of the subject, and the electrode device is adapted to be coupled to the vagus nerve.

In an embodiment, the control unit is adapted to:

drive the electrode device to apply the stimulating current, configured to induce the action potentials in an efferent therapeutic direction towards a heart of the subject, and
drive the electrode device to apply the inhibiting current, configured to inhibit the induced action potentials traveling in the efferent therapeutic direction in the first set of nerve fibers.

In an embodiment, the control unit is adapted to:

drive the electrode device to apply the stimulating current, configured to induce the action potentials in an afferent therapeutic direction towards a brain of the subject, and

drive the electrode device to apply the inhibiting current, configured to inhibit the induced action potentials traveling in the afferent therapeutic direction in the first set of nerve fibers.

In an embodiment, the nerve includes a surface set of fibers situated in a vicinity of an external surface of the nerve, and an axial set of fibers situated in a vicinity of a longitudinal axis of the nerve, and the control unit is adapted to drive the electrode device to apply the stimulating current to generate an electrical field defining a first activation function at the surface set of fibers, and defining a second activation function at the axial set of fibers, the first activation function less than about four times greater than the second activation function. For some applications, the control unit is adapted to configure the stimulating amplitude to be between about 1 and about 10 milliamps. For some applications, the control unit is adapted to configure the inhibiting amplitude to be between about 1 and about 10 milliamps.

There is still further provided, in accordance with an embodiment of the present invention, a treatment method, including:

applying an electrical current to a site of a subject identified as suffering from spontaneous atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject, and

configuring the current to treat the subject by maintaining the spontaneous AF for at least about 24 hours.

There is additionally provided, in accordance with an embodiment of the present invention, a treatment method, including:

identifying a subject suffering from spontaneous atrial fibrillation (AF);

applying a treatment to the subject; and

configuring the treatment to treat the subject by maintaining the spontaneous AF for at least about 24 hours.

There is yet additionally provided, in accordance with an embodiment of the present invention, a treatment method, including:

applying an electrical current to tissue of a subject; and

configuring the current to modify atrial motion of the subject to a level sufficient to reduce a risk of an occurrence of a thromboembolic event.

There is also provided, in accordance with an embodiment of the present invention, a treatment method, including:

applying an electrical current to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

repeatedly changing at least one parameter of the current, so as to restore normal sinus rhythm (NSR) of the subject.

There is further provided, in accordance with an embodiment of the present invention, a treatment method, including:

during a first period, pacing a heart of a subject suffering from atrial fibrillation (AF), and applying an electrical current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

during a second period following the first period, withholding applying the current to the site.

There is still further provided, in accordance with an embodiment of the present invention, a treatment method, including:

during a first period, pacing a heart of a subject suffering from atrial fibrillation (AF), and applying an electrical current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

detecting an occurrence of the AF; and

responsive to detecting the AF, during a second period following the first period, withholding applying the current to the site.

There is additionally provided, in accordance with an embodiment of the present invention, a treatment method, including:

identifying a subject suffering from atrial fibrillation (AF) principally caused by heightened adrenergic tone;

applying, to a site of the subject, which is capable of inducing action potentials in the site, the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the stimulating current to restore normal sinus rhythm (NSR) of the subject.

There is yet additionally provided, in accordance with an embodiment of the present invention, a method for use during defibrillation of a subject suffering from atrial fibrillation (AF), including:

applying an electrical current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the current to reduce pain experienced by the subject during the defibrillation, by causing bradycardia and a decreased level of alertness during the defibrillation.

There is also provided, in accordance with an embodiment of the present invention, a method for treating a subject suffering from atrial fibrillation (AF), including:

detecting the AF;

responsive to detecting the AF, applying an electrical current to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

during a first period beginning upon detecting the AF, configuring the current to attempt to restore normal sinus rhythm (NSR) of the subject;

determining whether NSR has been restored; and

during a second period beginning responsive to determining that NSR has not been restored within a threshold period of time after detecting the AF, configuring the current to maintain AF.

There is further provided, in accordance with an embodiment of the present invention, a method for stimulating a nerve of a subject, the nerve including a first set of fibers situated in a vicinity of an external surface of the nerve, and a second set of fibers situated in a vicinity of a longitudinal axis of the nerve, the method including applying to the nerve an electrical field defining a first activation function at the first set of fibers, and defining a second activation function at the second set of fibers, the first activation function less than about four times greater than the second activation function.

There is still further provided, in accordance with an embodiment of the present invention, a method for stimulating a nerve including small-, medium-, and large-diameter fibers, the method including:

applying a cathodic current to the nerve at a cathodic site of the nerve, so as to stimulate the nerve, the cathodic current having a cathodic amplitude sufficient to induce action potentials in the medium- and large-diameter fibers, but generally insufficient to induce action potentials in the small-diameter fibers; and

simultaneously applying to the nerve, at an anodal site of the nerve, an anodal current, which is capable of inhibiting action potentials in the nerve, the anodal current having an anodal amplitude sufficient to inhibit action potentials in the large-diameter fibers, but generally insufficient to inhibit action potentials in the medium-diameter fibers.

There is additionally provided, in accordance with an embodiment of the present invention, a method for stimulating a nerve, including:

applying to the nerve a stimulating current, which has a stimulating amplitude sufficient to induce action potentials in a first set and a second set of nerve fibers of the nerve, but not in a third set of nerve fibers of the nerve, the nerve fibers in the first set having generally larger diameters than the nerve fibers in the second set, and the nerve fibers in the second set having generally larger diameters than the nerve fibers in the third set; and

applying to the nerve an inhibiting current, which has an inhibiting amplitude sufficient to inhibit the induced action potentials in the first set of nerve fibers, but not in the second set of nerve fibers.

There is also provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply electrical stimulation to the site, and

configure the stimulation to prevent an occurrence of atrial fibrillation (AF).

For some applications, the control unit is configured to substantially continuously drive the electrode device to apply the stimulation during an application period lasting at least about 3 weeks. For some applications, in an operating mode of the control unit, the control unit is adapted to drive the electrode device to apply the stimulation during an application period lasting at least about 3 weeks, and to configure the stimulation such that, during the application period, a longest duration of time in which no stimulation is applied is less than 4 hours.

For some applications, the apparatus includes a sensor, adapted to sense a physiological parameter of the subject, and the control unit is adapted to drive the electrode device to apply the stimulation responsive to the sensed physiological parameter.

There is further provided, in accordance with an embodiment of the present invention, apparatus for treating a subject, including:

an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

a control unit, adapted to:

drive the electrode device to apply electrical stimulation to the site, and

configure the stimulation to reduce a probability of an occurrence of atrial fibrillation (AF).

For some applications, the control unit is configured to substantially continuously drive the electrode device to apply the stimulation during an application period lasting at least about 3 weeks. For some applications, in an operating mode of the control unit, the control unit is adapted to drive the electrode device to apply the stimulation during an application period lasting at least about 3 weeks, and to configure the stimulation such that, during the period, a longest duration of time in which no stimulation is applied is less than 4 hours.

For some applications, the apparatus includes a sensor, adapted to sense a physiological parameter of the subject, and the control unit is adapted to drive the electrode device to apply the stimulation responsive to the sensed physiological parameter.

There is still further provided, in accordance with an embodiment of the present invention, a method for treating a subject, including:

applying electrical stimulation to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the stimulation to prevent an occurrence of atrial fibrillation (AF).

There is additionally provided, in accordance with an embodiment of the present invention, a method for treating a subject, including:

applying an electrical stimulation to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and

configuring the stimulation to reduce a probability of an occurrence of atrial fibrillation (AF).

There is yet additionally provided, in accordance with an embodiment of the present invention, treatment apparatus, including:

an electrode device, adapted to be coupled to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

a pacing device, adapted to be applied to a heart of the subject; and

a control unit, adapted to:

drive the electrode device to apply an electrical current to the site,

drive the pacing device to apply a pacing signal to the heart, and

configure the current and the pacing signal so as to treat the AF.

For some applications, the control unit is adapted to configure the pacing signal to have a pulse repletion interval having a duration of between about 50% and about 200% of an atrial refractory period of the subject. For some applications, the control unit is adapted to configure the pacing signal to have a pulse repetition interval having a duration of between about 15 ms and about 190 ms.

For some applications, the control unit is adapted to configure the current to modulate an atrial refractory period of the subject.

For some applications, the control unit is adapted to modulate at least one parameter selected from the list consisting of: a parameter of the current, and a parameter of the pacing signal.

There is also provided, in accordance with an embodiment of the present invention, a treatment method, including:

applying an electrical current to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;

applying a pacing signal to a heart of the subject; and

configuring the current and the pacing signal so as to treat the AF.

There is also provided, in accordance with an embodiment of the present invention, a treatment method, including:

identifying a subject as one who is selected to undergo an interventional medical procedure; and

in response to the identifying, reducing a likelihood of a potential adverse effect of the procedure by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, the potential adverse effect includes an immune-mediated response to the procedure, and applying the current includes configuring the current to reduce the likelihood of the immune-mediated response.

In an embodiment, applying the current includes commencing applying the current within the first 7 days after the subject concludes undergoing the procedure. Alternatively, applying the current includes commencing applying the current during the procedure. Further alternatively, applying the current includes commencing applying the current within a three week period that begins one week before the subject begins undergoing the procedure.

In an embodiment, the interventional procedure includes a heart procedure, and identifying the subject includes identifying the subject as one who is selected to undergo the heart procedure. For some applications, applying the current includes configuring the current to reduce mechanical stress of the heart. Alternatively or additionally, applying the current includes configuring the current to reduce a heart rate of the subject. Further alternatively or additionally, applying the current includes configuring the current to improve coronary blood flow of the subject.

In an embodiment, the heart procedure includes coronary bypass surgery, and identifying the subject includes identifying the subject as one who is selected to undergo the coronary bypass surgery. For some applications, applying the current includes configuring the current to reduce a likelihood of postoperative atrial fibrillation. Alternatively or additionally, applying the current includes configuring the current to reduce a likelihood of graft failure. Further alternatively or additionally, applying the current includes configuring the current to reduce a likelihood of a reduction of peripheral blood flow.

In an embodiment, the heart procedure includes carotid endarterectomy, and identifying the subject includes identifying the subject as one who is selected to undergo the carotid endarterectomy. For some applications, applying the current includes configuring the current to reduce a likelihood of restenosis. Alternatively or additionally, applying the current includes configuring the current to reduce a likelihood of intra-operative stroke.

In an embodiment, the interventional procedure includes a surgical procedure, and identifying the subject includes identifying the subject as one who is selected to undergo the surgical procedure. For some applications, the surgical procedure includes a surgical heart procedure, and identifying the subject includes identifying the subject as one who is selected to undergo the surgical heart procedure.

For some applications, the surgical procedure includes an abdominal surgical procedure, and identifying the subject includes identifying the subject as one who is selected to undergo the abdominal surgical procedure. For some applications, applying the current includes configuring the current to reduce a likelihood of a complication selected from the group consisting of: stenosis of gastrointestinal (GI) tract segments involved in the surgical procedure, GI stasis, and flare of inflammatory disease.

In an embodiment, the surgical procedure includes transplantation of tissue selected from the group consisting of: an organ and cells, and identifying the subject includes identifying the subject as one who is selected to undergo the transplantation of the selected tissue.

In an embodiment, the surgical procedure includes implantation of an implantable medical device, and identifying the subject includes identifying the subject as one who is selected to undergo the implantation of the device.

In an embodiment, the surgical procedure includes a heart transplantation procedure, and identifying the subject includes identifying the subject as one who is selected to undergo the heart transplantation procedure. For some applications, applying the current includes applying the current beginning no earlier than 7 days prior to the heart transplantation procedure, and concluding no later than 7 days after the heart transplantation procedure. Alternatively, applying the current includes applying the current beginning at least 2 weeks prior to the heart transplantation procedure. Further alternatively, applying the current includes concluding application of the current at least 2 weeks after the heart transplantation procedure.

In an embodiment, the surgical procedure includes a cardiac procedure selected from the group consisting of: a valve replacement procedure, and a valvoplasty procedure, and identifying the subject includes identifying the subject as one who is selected to undergo the selected cardiac procedure.

In an embodiment, the surgical procedure includes a percutaneous transluminal coronary angioplasty (PTCA) procedure, and identifying the subject includes identifying the subject as one who is selected to undergo the PTCA procedure. For some applications, the potential adverse effect includes restenosis, and reducing the likelihood includes reducing the likelihood of the restenosis.

There is also provided, in accordance with an embodiment of the present invention, a treatment method, including:

determining that a subject suffers from an unwanted habitual behavior;

designating the subject for treatment of the behavior, responsively to the determination; and

reducing at least one parameter of the behavior selected from the group consisting of: a rate of occurrence of the behavior, and a level of intensity of the behavior, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes applying the current in response to an indication from the subject that the subject is experiencing a desire to perform the unwanted habitual behavior.

For some applications, applying the current includes applying the current at non-constant intervals. For some applications, applying the current at the non-constant intervals includes applying the current at intervals selected from the group consisting of: random intervals, quasi-random intervals, and seemingly random intervals.

There is further provided, in accordance with an embodiment of the present invention, a treatment method, including:

determining that a subject suffers from an obsessive compulsive disorder;

designating the subject for treatment of the obsessive compulsive disorder, responsively to the determination; and

reducing at least one parameter of the disorder selected from the group consisting of: a rate of occurrence of a symptom of the disorder, and a level of intensity of the disorder, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is still further provided, in accordance with an embodiment of the present invention, a treatment method, including:

determining that a subject smokes;

designating the subject for treatment of the smoking, responsively to the determination; and

reducing a rate of occurrence of the smoking by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes applying the current in response to an indication from the subject that the subject is experiencing a desire to smoke.

There is yet further provided, in accordance with an embodiment of the present invention, a treatment method, including:

determining that a subject suffers from an addiction to a drug;

designating the subject for treatment of the addiction, responsively to the determination; and

reducing at least one parameter of the addiction selected from the group consisting of: a rate of occurrence of use of the drug, and a level of intensity of use of the drug, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, the drug includes nicotine, and determining includes determining that the subject suffers from the addiction to nicotine.

For some applications, applying the current includes applying the current in response to an indication from the subject that the subject is experiencing a desire to administer the drug.

There is additionally provided, in accordance with an embodiment of the present invention, a treatment method, including:

determining that a subject suffers from Tourette syndrome;

designating the subject for treatment of the syndrome, responsively to the determination; and

reducing at least one parameter of the syndrome selected from the group consisting of: a rate of occurrence of the syndrome, and a level of intensity of a symptom of the syndrome, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is yet additionally provided, in accordance with an embodiment of the present invention, a treatment method, including:

determining that a subject suffers from a sleep disorder;

designating the subject for treatment of the sleep disorder, responsively to the determination; and

reducing at least one parameter of the disorder selected from the group consisting of: a rate of occurrence of the disorder, and a level of intensity of the disorder, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

In an embodiment, the sleep disorder includes sleep apnea, and determining includes determining that the subject suffers from the sleep apnea.

In an embodiment, the sleep disorder includes insomnia, and determining includes determining that the subject suffers from the insomnia, and applying the current includes configuring the current to improve at least one parameter of sleep of the subject selected from the group consisting of: quality of sleep, and duration of sleep. For some applications, applying the current includes applying the current in response to an indication from the subject that the subject is experiencing difficulty sleeping.

There is still additionally provided, in accordance with an embodiment of the present invention, a treatment method, including:

determining that a subject suffers from insulin resistance;

designating the subject for treatment of the insulin resistance, responsively to the determination; and

reducing the insulin resistance by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes configuring the current to reduce short-term sensitivity of muscle tissue to insulin.

There is further provided, in accordance with an embodiment of the present invention, a treatment method, including:

determining that a subject suffers from renal failure;

designating the subject for treatment of the renal failure, responsively to the determination; and

improving renal function of the subject by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes configuring the current to increase a glomerular filtration rate (GFR) of the subject by acting on a kidney vascular bed.

For some applications, applying the current includes configuring the current to reduce blood flow to skeletal muscle of the subject. For some applications, applying the current includes applying the current during a period of time selected from the group consisting of: a period when the subject is sleeping, and a period during which the subject is physically inactive.

For some applications, the method includes receiving a signal from the subject signifying that the subject is undergoing dialysis, and applying the current includes applying the current responsively to the received signal.

There is still further provided, in accordance with an embodiment of the present invention, a treatment method, including:

determining that a subject suffers from hepatic failure;

designating the subject for treatment of the hepatic failure, responsively to the determination; and

improving hepatic function of the subject by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes configuring the current to increase blood flow through a portal vein of the subject by reducing blood flow to skeletal muscle.

For some applications, applying the current includes applying the current during a period of time selected from the group consisting of: a period when the subject is sleeping, and a period during which the subject is physically inactive.

There is additionally provided, in accordance with an embodiment of the present invention, a treatment method, including:

determining that a subject suffers from a symptom of muscle fatigue;

designating the subject for treatment of the muscle fatigue, responsively to the determination; and

reducing a level of severity of the symptom of muscle fatigue by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is yet additionally provided, in accordance with an embodiment of the present invention, a treatment method, including:

determining that a subject suffers from at least one condition selected from the group consisting of: impaired sexual function, and impaired sexual pleasure;

designating the subject for treatment of the condition, responsively to the determination; and

improving the condition by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is still additionally provided, in accordance with an embodiment of the present invention, a treatment method, including:

determining that a subject suffers from anemia;

designating the subject for treatment of the anemia, responsively to the determination; and

promoting red blood cell production by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is also provided, in accordance with an embodiment of the present invention, a treatment method, including:

determining that a subject suffers from reduced peripheral blood flow;

designating the subject for treatment of the reduced peripheral blood flow, responsively to the determination; and

applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is further provided, in accordance with an embodiment of the present invention, a treatment method, including:

determining that a subject has suffered a cerebrovascular accident (CVA);

designating the subject for treatment of the CVA, responsively to the determination; and

reducing a level of damage due to the CVA by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is still further provided, in accordance with an embodiment of the present invention, a treatment method, including:

determining that a subject suffers from Attention Deficit Hyperactivity Disorder (ADHD);

designating the subject for treatment of the ADHD, responsively to the determination; and

reducing at least one symptom of the ADHD by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

For some applications, applying the current includes configuring the current to reduce the symptom by reducing hyperactivity or activity of brain cells of the subject.

There is yet further provided, in accordance with an embodiment of the present invention, a treatment method, including:

determining that a subject has suffered a stroke;

designating the subject for treatment of the stroke, responsively to the determination; and

treating the stroke by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject.

For some applications, applying the current includes configuring the current to treat the stroke by reducing hyperactivity or activity of brain cells of the subject.

For some applications, applying the current includes configuring the current to reduce secondary stroke damage to cells in areas adjacent to a hypoxic area by reducing cell activity in the areas. Alternatively or additionally, applying the current includes configuring the current to reduce the likelihood of an immune-mediated response to the stroke.

There is also provided, in accordance with an embodiment of the present invention, a treatment method, including:

determining that a subject suffers from a condition selected from the group consisting of: an allergy, an allergic reaction, and multiple sclerosis;

designating the subject for treatment of the selected condition, responsively to the determination; and

reducing at least one symptom of the condition by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is additionally provided, in accordance with an embodiment of the present invention, a treatment method, including:

determining that a subject suffers from a condition selected from the group consisting of: vasculitis, Wegener granulomatosis, temporal arteritis, Takayasu arteritis, polyarteritis nodosa, systemic sclerosis, systemic lupus erythematosus, flare of Crohn's disease, flare of ulcerative colitis, autoimmune hepatitis, glomerulonephritis, arthritis, reactive arthritis, rheumatoid arthritis, pancreatitis, thyroiditis, idiopathic thrombocytopenic purpura (ITP), thrombotic thrombocytopenic purpura (TTP), multi-organ failure associated with sepsis, anaphylactic shock, Acute Respiratory Distress Syndrome (ARDS), and asthma;

designating the subject for treatment of the selected condition, responsively to the determination; and

reducing immune system hyperactivation associated with the selected condition by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

There is yet additionally provided, in accordance with an embodiment of the present invention, a treatment method, including:

determining that a subject suffers from a condition;

designating the subject for treatment of the condition by regulation of cell division of the subject, responsively to the determination; and

treating the condition by regulating the cell division of the subject by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject.

In an embodiment, applying the current includes configuring the current to increase cell division of the subject. For example, the condition may be associated with improperly-regulated cell division, and determining may include determining that the subject suffers from the condition associated with improperly-regulated cell division. Alternatively or additionally, the condition is selected from the group consisting of: anemia, a neurodegenerative disease, liver cirrhosis, an immune deficiency, a skin burn, a skin abrasion, a muscle degenerative disorder, cardiac failure, and a reproductive system disorder, and determining includes determining that the subject suffers from the selected condition.

In an embodiment, applying the current includes configuring the current to decrease cell division of the subject. For some applications, the condition is selected from the group consisting of: a neoplastic disorder, a hematologic malignancy, and polycythemia vera, and determining includes determining that the subject suffers from the selected condition.

There is still additionally provided, in accordance with an embodiment of the present invention, a treatment method, including:

determining that a subject suffers from hiccups;

designating the subject for treatment of the hiccups, responsively to the determination; and

reducing at least one parameter of the hiccups selected from the group consisting of: a rate of occurrence of the hiccups, and a level of intensity of the hiccups, by applying an electrical current to a parasympathetic site of the subject selected from the group consisting of: a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, and a right ventricle of the subject.

There is yet additionally provided, in accordance with an embodiment of the present invention, apparatus for treating a subject, including:

an electrode device, adapted to be coupled to a parasympathetic site of the subject selected from the group consisting: of a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject; and

a control unit, adapted to:

drive the electrode device to apply a current to the site,

receive a sensed physiological value of the subject selected from the group consisting of: a temperature of the subject, a blood glucose level of the subject, a blood lipid level of the subject, a blood lactic acid level of the subject, a blood CO2 level of the subject, a blood O2 level of the subject, a blood urea level of the subject, a blood creatinine level of the subject, and a blood ammonia level of the subject, and

set at least one parameter of the applied current responsively to the sensed physiological value.

For some applications, the control unit is adapted to configure the applied current to reduce a heart rate of the subject.

There is still additionally provided, in accordance with an embodiment of the present invention, a method for treating a subject, including:

applying a current to a parasympathetic site of the subject selected from the group consisting: of a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject;

receiving a sensed physiological value of the subject selected from the group consisting of: a temperature of the subject, a blood glucose level of the subject, a blood lipid level of the subject, a blood lactic acid level of the subject, a blood CO2 level of the subject, a blood O2 level of the subject, a blood urea level of the subject, a blood creatinine level of the subject, and a blood ammonia level of the subject; and

setting at least one parameter of the applied current responsively to the sensed physiological value.

There is yet additionally provided, in accordance with an embodiment of the present invention, apparatus including an electrode assembly adapted to be coupled to nervous tissue of a subject, the electrode assembly including one or more conductive elements, and at least a portion of the electrode assembly is adapted to be dissolvable after the electrode assembly has been coupled to the tissue.

In an embodiment, the nervous tissue includes a nerve of the subject, and the electrode assembly is adapted to be coupled to the nerve.

In an embodiment, the electrode assembly is adapted to come loose from the tissue upon dissolving of the dissolvable at least a portion thereof.

For some applications, the dissolvable at least a portion of the electrode assembly includes a material selected from the group consisting of: polyglycolic acid (PGA), and poly(L-lactide) acid (PLL).

For some applications, when the electrode assembly is coupled to the tissue, a portion of the electrode assembly is positioned within 2 cm of the tissue, and the portion does not include any metal components. For some applications, the electrode assembly includes electrode leads including metal wires, and the electrode assembly is configured such that the metal wires are not positioned within 2 cm of the tissue when the electrode assembly is coupled to the tissue.

In an embodiment, the electrode assembly includes electrode leads, and when the electrode assembly is coupled to the tissue, at least a portion of the electrode leads are positioned within 2 cm of the tissue, and the portion of the electrode leads includes tubes including an electrically conductive biologically-compatible liquid.

For some applications, the apparatus includes a control unit, adapted to measure an impedance of the electrode assembly, and to determine, responsively to the measured impedance, whether the dissolvable at least a portion of the electrode assembly has dissolved sufficiently to enable safe removal of the electrode assembly from the subject.

There is also provided, in accordance with an embodiment of the present invention, a method including coupling an electrode assembly to nervous tissue of a subject, the electrode assembly including one or more conductive elements, and at least a portion of the electrode assembly is adapted to be dissolvable after the electrode assembly has been coupled to the tissue.

For some applications, the method includes removing a non-dissolvable portion of the electrode assembly from the tissue upon dissolving of the dissolvable at least a portion thereof.

The present invention will be more fully understood from the following detailed description of embodiments thereof, taken together with the drawings, in which:

### BRIEF DESCRIPTION OF THE DRAWINGS

FIG. 1 is a schematic illustration of apparatus for treating a subject, in accordance with an embodiment of the present invention;

FIG. 2A is a simplified cross-sectional illustration of a multipolar electrode device applied to a vagus nerve, in accordance with an embodiment of the present invention;

FIG. 2B is a simplified cross-sectional illustration of a generally-cylindrical electrode device applied to a vagus nerve, in accordance with an embodiment of the present invention;

FIG. 2C is a simplified perspective illustration of the electrode device of FIG. 2A, in accordance with an embodiment of the present invention;

FIG. 3 is a simplified perspective illustration of a multipolar point electrode device applied to a vagus nerve, in accordance with an embodiment of the present invention;

FIG. 4 is a conceptual illustration of the application of current to a vagus nerve, in accordance with an embodiment of the present invention;

FIG. 5 is a simplified illustration of an electrocardiogram (ECG) recording and of example timelines showing the timing of the application of a series of stimulation pulses, in accordance with an embodiment of the present invention;

FIG. 6 is a schematic illustration of a series of bursts, in accordance with an embodiment of the present invention;

FIG. 7 is a schematic illustration of a stimulation regimen, in accordance with an embodiment of the present invention;

FIG. 8 is a schematic illustration of a stimulation regimen, in accordance with an embodiment of the present invention;

FIGS. 9 and 10 are graphs showing in vivo experimental results measured in accordance with an embodiment of the present invention;

FIG. 11 is a chart showing in vivo experimental results in accordance with an embodiment of the present invention;

FIGS. 12A and 12B are graphs showing an analysis of the experimental results of the experiment of FIG. 10, in accordance with an embodiment of the present invention;

FIGS. 13A and 13B are graphs showing in vivo experimental results in accordance with an embodiment of the present invention; and

FIG. 14 is a flow chart that schematically illustrates a method for determining and applying an appropriate AF treatment based on a countdown, in accordance with an embodiment of the present invention.

### DETAILED DESCRIPTION OF EMBODIMENTS

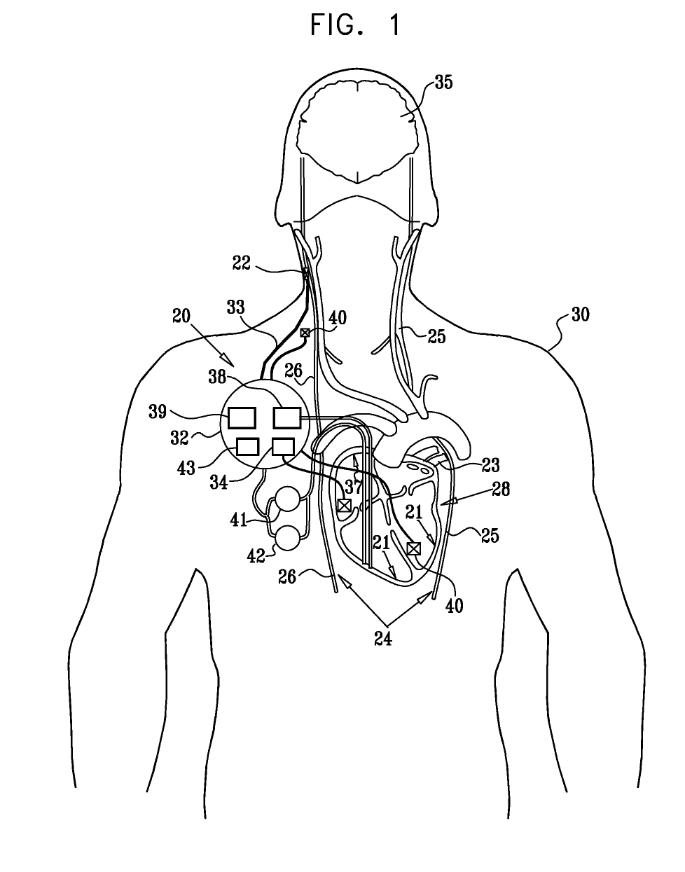

FIG. 1 is a schematic illustration of apparatus 20 for treating a subject 30, in accordance with an embodiment of the present invention. Apparatus 20 comprises at least one electrode device 22, which is applied to a site of the subject selected from the group consisting of: a vagus nerve 24 (either a left vagus nerve 25 or a right vagus nerve 26), which innervates a heart 28 of subject 30, an epicardial fat pad (e.g., a sinoatrial (SA) node fat pad, or an atrioventricular (AV) node fat pad), a pulmonary vein, a carotid artery, a carotid sinus, a coronary sinus, a vena cava vein, a jugular vein, an azygos vein, an innominate vein, and a subclavian vein. Alternatively or additionally, the site is selected from the group consisting of: a right ventricle, a right atrium, and other parasympathetic tissue that innervates heart 28. “Vagus nerve,” and derivatives thereof, as used in the present application including the claims, is to be understood to include portions of the left vagus nerve, the right vagus nerve, and branches of the vagus nerve such as the cervical or thoracic vagus nerve, superior cardiac branch, and inferior cardiac branch.

Apparatus 20 further comprises an implanted or external control unit 32, which typically communicates with electrode device 22 over a set of leads 33. For some applications, apparatus 20 comprises two electrode devices 22, one of which is applied to left vagus nerve 25, and the other to right vagus nerve 26. Alternatively or additionally, apparatus 20 comprises an electrical stimulator 34, which typically comprises one or more electrodes, and which is adapted to electrically stimulate tissue of patient 30, such as cardiac tissue, epicardial fat pads, atrial tissue 37, ventricular tissue 21, pulmonary venous tissue 23, the carotid artery, the internal jugular vein, the carotid sinus, or the vena cava vein.

Control unit 32 is adapted to drive electrode device 22 to apply signals to the site, and to configure the current to stimulate autonomic nervous tissue in the site. The control unit typically configures the applied signals to induce the propagation of efferent nerve impulses towards heart 28. The control unit configures the signals based on the particular application, by setting one or more parameters of the signals, such as:
frequency of pulses within a pulse burst, e.g., for n pulses during a burst lasting t milliseconds, the burst has a frequency of 1000 n/t Hz;
amplitude;
pulse width;
number of pulse delivered per heartbeat (pulses per trigger, or PPT);
duty cycle;
pulse polarity; and
timing within the cardiac cycle.

In an embodiment of the present invention, a method for treating subject 30 who is at risk of suffering from atrial fibrillation (AF) comprises reducing a risk of an occurrence of an episode of the AF by applying an electrical current to a site of subject 30 selected from the group consisting of: vagus nerve 24 (either left vagus nerve 25 or right vagus nerve 26), an epicardial fat pad, a pulmonary vein, a carotid artery, a carotid sinus, a coronary sinus, a vena cava vein, a jugular vein, an azygos vein, an innominate vein, and a subclavian vein. Alternatively or additionally, the site is selected from the group consisting of: a right ventricle, a right atrium, and other parasympathetic tissue that innervates heart 28. For some applications, control unit 32 of apparatus 20 drives electrode device 22 to apply the electrical current.

For some applications, the current is applied intermittently during alternating “on” and “off” periods. Typically, each of the “on” periods has an “on” duration equal to at least 1 second (e.g., between 1 and 10 seconds, such as about 3 seconds), and each of the “off” periods has an “off” duration equal to at least 50% of the “on” duration, e.g., at least 100% or 200% of the “on” duration, such as about 9 seconds.

For some applications, control unit 32 is configured to apply the current on a chronic, long-term basis, even when the subject is not currently experiencing an episode of the AF, and even in the absence of a prediction of an imminent episode of the AF. The current is thus typically applied during normal sinus rhythm (NSR). For some applications, chronically applying the current comprises applying the current at least once during each of seven consecutive 48-hour periods, such as at least once during each of 14 consecutive 24-hour periods, or at least once during each of 28 consecutive 12-hour periods. For some applications, applying the current comprises applying at least 100 pulses of the current per day.

For some applications, chronically applying the current comprises applying the current at least once per day during a three-week period. For example, apparatus 20 may be implanted and configured to apply the current for a period of at least three months, a year, or three years, which period includes at least one three-week period during which the current is applied at least once per day, e.g., at least twice per day, and/or for at least 30 minutes per day, such as at least 60 minutes per day. Alternatively, the current is applied chronically, but less frequently, such as at least once every 48 hours, at least twice per week, or at least once per week. For some applications, applying the current at least once per day comprises applying at least a total 100 pulses per day.

In an embodiment, apparatus 20 comprises a sensor adapted to detect normal sinus rhythm (NSR) and generate a sensor signal responsive thereto, and control unit 32 is adapted to receive the sensor signal, and to drive electrode device 22 to apply the current responsive to the sensor signal.

In an embodiment of the present invention, subject 30 is determined to be at risk of suffering from AF by identifying that the subject suffers from at least one of the following conditions:
paroxysmal AF;
self-terminating AF episodes;
an enlarged atrium;
multiple atrial premature beats (APBs);
mitral stenosis;
heart failure;
thyrotoxicosis;
hypertension; and
atrial flutter.

Alternatively or additionally, the subject is determined to be at risk of suffering from AF by identifying that the subject has undergone an interventional heart procedure, such as coronary bypass surgery or valve replacement surgery.

For some applications, this determination is made after the subject has suffered from at least one episode of the AF, while for other applications, the determination is made prior to the subject suffering from any known episodes of the AF. Typically, the identification that the subject is at risk is made by a medical professional. Typically, reducing the risk comprises reducing the risk in the absence of a determination by any device directly or indirectly coupled to the electrode device that the subject is at risk of suffering from the AF. In other words, the medical decision to implant apparatus 20 is typically made by a medical professional who identifies that the subject is at risk of suffering from AF, but apparatus 20 itself does not assess the subject's risk of suffering from AF, or any episodes or particular episode thereof.

For some applications, control unit 32 applies the current not responsively to any physiological parameters sensed by any device coupled to electrode device 22 or to control unit 32 (e.g., the control unit itself or a sensor coupled to the control unit).

For some applications, the control unit applies the current not responsively to any measure of a heart rate of the subject (which may be expressed as a heart rate or interval, e.g., an R-R interval) determined by the control unit. For these application, control unit 32 does not configure any parameters of the applied current responsively to any measure of the heart rate, including any timing parameters of the current application. For these applications, although the control unit does not apply the current responsively to the measure of the heart rate, the control unit may apply the current responsively to other physiological measures, such as described herein. For example, the control unit may synchronize the applied current to one or more features of a cardiac cycle of the subject, such as described herein.

Control unit 32 typically does not configure the current to achieve regulation of a heart rate of the subject, such as to achieve a target heart rate or range. For some applications, the current is configured to minimize an effect of the applying of the current on a heart rate of the subject, as described hereinbelow.

For some applications, control unit 32 is adapted to receive feedback from one or more of the electrodes in electrode device 22, and to regulate the signals applied to the electrode device responsive thereto.

Alternatively, control unit 32 is configured to receive and analyze one or more sensed physiological parameters or other parameters of subject 30, such as ventricular and/or atrial rate, electrocardiogram (ECG), blood pressure, indicators of decreased cardiac contractility, cardiac output, norepinephrine concentration, left ventricular end diastolic pressure (LVEDP), baroreflex sensitivity, or motion of the subject. In order to receive these sensed parameters, control unit 32 may comprise, for example, an ECG monitor 38, connected to a site on the subject's body such as heart 28, for example using one or more subcutaneous sensors or ventricular and/or atrial intracardiac sensors. The control unit may also comprise an accelerometer 39 for detecting motion of the subject. Alternatively, ECG monitor 38 and/or accelerometer 39 comprise separate implanted devices placed external to control unit 32, and, optionally, external to the subject's body. Alternatively or additionally, control unit 32 receives signals from one or more physiological sensors 40, such as blood pressure sensors. Sensors 40 are typically implanted in the subject, for example in a left ventricle of the heart. For example, sensors 40 may comprise a pressure gauge for measuring LVEDP, which gauge may be adapted to be placed in the left ventricle, a left atrium of the heart, or in a pulmonary artery. For some applications, control unit 32 comprises or is coupled to an implantable cardioverter defibrillator (ICD) 41 and/or a pacemaker 42 (e.g., a bi-ventricular or standard pacemaker).

In an embodiment of the present invention, control unit 32 drives electrode device 22 to apply an electrical current to vagus nerve 24, and drives pacemaker 42 to apply pacing signals to heart 28. The control unit configures the current and the pacing signals to treat the AF of patient 30. For some applications, the control unit configures pacemaker 42 to apply the pacing signals with pulse repetition intervals having a duration of between about 50% and about 200% of an atrial refractory period of patient 30 (e.g., between about 15 ms and about 190 ms), so as to treat the AF. For some applications, the control unit configures the vagal stimulation current to modulate the atrial refractory period. For some applications, the control unit modulates one or more parameters of the vagal stimulation current and/or of the pacing signal, such as on/off time, amplitude, number of pulses, pulse repetition interval (i.e., the interval between the leading edges of two consecutive pulses), or other parameters described herein.

In some embodiments of the present invention, upon sensing an occurrence of an episode of the AF, the control unit reduces a strength of the current, e.g., withholds applying the current. The inventors believe that application of the current sometimes prolongs episodes of the AF, so reducing the strength of or withholding the current generally allows episodes to resolve more quickly than they would during application of the current at full strength. Similarly, for some applications, upon predicting an imminent episode of the AF, the control unit reduces the strength of the current, e.g., withholds applying the current. For some applications, techniques for sensing or predicting the imminent episode of the AF are used that are described in U.S. Pat. No. 5,522,854 to Ideker et al., U.S. Pat. No. 5,658,318 to Stroetmann et al., U.S. Pat. No. 7,050,846 to Sweeney et al., and/or U.S. Pat. No. 5,578,061 to Stroetmann et al., all of which are incorporated herein by reference.

For some applications, control unit 32 senses the occurrence of the episode of AF (and/or distinguishes between AF and NSR) by analyzing an ECG signal generated by ECG monitor 38. In order to detect rapid atrial activity indicative of AF, the analysis may include one or more of the following:
P-wave analysis;
analysis of ventricular response rate and/or ventricular response variability;
sensed pressure, such as atrial pressure, sensed venous pressure, and/or sensed arterial pressure;
the relationship(s) between one or more of the sensed pressures and sensed ventricular contractions (in the case of arterial pressure, such relationship is an indication of pulse deficit); and/or
analysis of the duration of the isoelectrical segment of the ECG, optionally using the technique described in an article by Wijffels M C et al., entitled, “Atrial fibrillation begets atrial fibrillation,” Circulation 92:1954-1968 (1995), which is incorporated herein by reference. A duration greater than a first threshold value is typically indicative of NSR, while a duration less than a second threshold value, the second threshold value less than or equal to the first threshold value, is typically indicative of AF.

Control unit 32 itself may perform this analysis, or it may transmit data for analysis by an external processor (not shown).

Typically, apparatus 20 is programmable by a physician, such as by using an external console wirelessly in communication with control unit 32. For some applications, the apparatus provides notification of various occurrences, such as the initiation of AF, the initiation of treatment, or a mechanical failure. The apparatus may provide such notifications by various means, including generating a tone, vibrating, and/or wirelessly communicating with a local or remote receiver, such as one located at a medical facility.

In an embodiment of the present invention, apparatus 20 comprises a sensing unit configured to detect whether applying the current causes one or more cardiac contractions, and control unit 32 is configured, responsively to finding that applying the current causes the contractions, to reduce a strength of the current to a level insufficient to cause the contractions. Typically, the sensing unit comprises ECG monitor 38.

In an embodiment of the present invention, upon sensing an occurrence of an episode of the AF, control unit 32 reduces a strength of the current, e.g., withholds applying the current, typically during a strength reduction period having a duration of at least one minute, e.g., at least 5 minutes, at least 10 minutes, at least 20 minutes, or at least one hour. The inventors believe that application of the current sometimes prolongs episodes of AF, so reducing the strength of or withholding the current generally allows episodes to resolve more quickly than they would during application of the current at full strength. Similarly, for some applications, upon predicting an imminent episode of the AF, control unit 32 reduces the strength of the current, e.g., withholds applying the current. For some applications, upon conclusion of the strength reduction period, the control unit configures the current to reduce a heart rate of the subject if the episode of AF has not terminated, and the subject has an elevated heart rate.

In an embodiment, control unit 32 is configured to apply the current during an episode of the AF, and is not configured to configure the current to resolve the episode. For some applications, the control unit is configured to apply the current during the episode and during at least one period not during the episode. For some applications, the control unit is configured to detect the episode, and to apply the current responsively to the detecting. For some applications, the control unit is configured to apply the current even during an episode of the AF, without configuring the current to resolve the episode.

In some embodiments of the present invention, control unit 32 applies the current in a series of bursts, each of which bursts includes at least one pulse. For some applications, the control unit synchronizes at least a portion of the bursts with a feature of a cardiac cycle of the subject, such as a P-wave or R-wave. Synchronization with the P-wave has the effect of automatically withholding stimulation during AF, because no P-wave is present during AF.

For some applications, control unit 32 applies the signals to the selected parasympathetic site in a series of bursts, each of which bursts includes at least one pulse. For some of these applications, during periods in which stimulation is being applied, one burst is applied during each cardiac cycle, or during every nth cardiac cycle, such as one burst every second or every third cardiac cycle, with one or more of the following parameters (collectively, these parameters are referred to hereinbelow as “typical stimulation parameters”):
Timing of the stimulation: for example, each pulse may be initiated at about 100 milliseconds after an R-wave.
Pulse duration: each pulse typically has a duration of between about 100 microseconds and about 2.5 milliseconds, e.g., about 1 millisecond.
Pulse amplitude: the pulses are typically applied with an amplitude of between about 0.1 and about 9 mA, e.g., about 2.5 mA.
Pulse repetition interval (PRI): the pulses within the burst of pulses typically have a PRI (the time from the initiation of a pulse to the initiation of the following pulse) of, on average, at least 20 ms, such as at least 30 ms, e.g., at least 50 ms or at least 75 ms; alternatively, the PRI may be between about 1 and about 20 milliseconds, e.g., about 6 milliseconds.
Pulses per trigger (PPT): the burst of pulses typically contains between about 1 and about 10 pulses, e.g., 3 pulses or 4 pulses.
Pulse period, i.e., burst duration (equal to the product of PRI and PPT): the burst of pulses typically has a total duration of between about 1 and about 180 milliseconds.
Duty cycle: stimulation is typically applied once per heartbeat, once every second heartbeat, or once every third heartbeat.
On/off status: for some applications, stimulation is always “on”, i.e., constantly applied (in which case, parameters closer to the lower ends of the ranges above are typically used). For other applications, on/off cycles vary between a few seconds to several minutes, e.g., “on” for 15 seconds, “off” for 60 seconds.

Alternatively, the stimulation is not synchronized with the cardiac cycle. For some non-synchronized applications, the applicable parameters listed above are used, such as a PPT of 3 or 4 pulses. For some applications, the bursts are applied at a frequency (i.e., bursts per second) of 1 Hz or less, e.g., 0.5 Hz or less.

In an embodiment of the present invention, a method for enhancing or sustaining the efficacy of drug treatment for atrial fibrillation (AF) comprises administering a drug to subject 30 and applying signals to a site, such as the vagus nerve, that innervates heart 28 of the subject, such as described in the above-mentioned U.S. application Ser. No. 10/866,601. The drug administered typically includes either:
a sinus rhythm maintenance drug (i.e., an antiarrhythmic drug), such as a beta-blocker, digoxin, amiodarone, disopyramide, dofetilide, a class IC drug (e.g., flecamide, propafenone), procainamide, quinidine, or sotalol; or
a ventricular rate control drug, such as a beta-blocker (e.g., esmolol), calcium channel antagonists (e.g., verapamil, diltiazem), or digoxin.

According to this method, the efficacy of the drug is typically enhanced or sustained by (a) configuring the signals so as to prevent electrical remodeling of the atria, which remodeling generally reduces drug effectiveness over time, (b) configuring the signals so as to achieve a therapeutic benefit similar to that of the drug, which typically results in a synergistic effect between the therapeutic benefit of the drug and the vagal stimulation, and/or (c) configuring the signals so as to reduce the mechanical tension on the atria.

Atrial electrical remodeling, i.e., electrophysiological changes to the atria, commonly occurs in subjects suffering from AF. Such electrical remodeling is believed to be caused by the underlying heart condition that instigated the AF, and/or by the effect of the AF itself on the atria (see the above-mentioned article by Wijffels M C et al., entitled “Atrial fibrillation begets atrial fibrillation”). As electrical remodeling becomes more severe, relapses into AF become more frequent and difficult to prevent. As a result, drug therapy for preventing such relapses becomes less effective. Vagal or other parasympathetic stimulation, using techniques described herein, typically delays or prevents (i.e., delays indefinitely) electrical remodeling. For subjects also receiving antiarrhythmic drug therapy, such delaying generally prolongs the effectiveness of the drug therapy. For some applications, control unit 32 configures the signals applied to the site using parameters described hereinbelow for applying vagal or other parasympathetic stimulation with minimum heart rate reduction.

For some applications, control unit 32 configures the current to reduce mechanical stress of heart 28, and/or to induce rhythmic vagal activity. Such rhythmic, synchronized vagal activity generally mimics normal vagal traffic, which is sometimes reduced in these subjects (who may, for example, suffer from heart failure or hypertension). Stable NSR typically results from such treatment, thereby generally reducing the occurrence of AF.

For example, stimulation may be applied by cycling between a first set and a second set of parameters, applying each set for less than about 15 seconds, e.g., for between about 1 and about 4 seconds. The first set of parameters may include: (a) a low amplitude, e.g., 2 mA, so as to recruit a relatively small number of nerve fibers, (b) optional synchronization with inhalation, and (c) one pulse per trigger (PPT), for example applied at about 300 milliseconds after an R-wave. The second set of parameters may include: (a) a greater amplitude, e.g., 3 mA, so as to recruit a greater number of fibers, (b) optional synchronization with exhalation, and (c) three PPT, applied at about 300 milliseconds after an R-wave. Both sets of parameters optionally include a pulse width of about 1 millisecond and/or a PRI that is on average at least 20 ms, such as at least 30 ms, e.g., at least 50 ms or at least 75 ms; alternatively, the PRI may be between about 4 and about 20 ms.

In an embodiment of the present invention, vagal stimulation is applied in combination with administration of a drug, as described in the following examples:

In Combination with Beta-Blockers

A beta-blocker is administered substantially at its usual dosage (i.e., at a dosage determined independently of applying the vagal stimulation), and vagal stimulation is applied using parameters described hereinbelow for applying vagal stimulation with minimum heart rate reduction.

### For Bradycardia

For treating a patient susceptible to bradycardia, a beta-blocker is administered at a dosage lower than would normally be indicated, and vagal stimulation is applied using parameters described hereinbelow for applying vagal stimulation with minimum heart rate reduction, or using parameters at the lower range of the typical stimulation parameters described hereinabove. Upon detection of bradycardia, the vagal stimulation is terminated.

In Combination with a Sinus Rhythm Maintenance Drug

A patient who suffers from AF is treated by conventional cardioversion and a sinus rhythm maintenance drug, such as quinidine. To enhance the desired effect of the drug, the drug is administered in conjunction with the application of rhythmic vagal stimulation. The resulting rhythmic, synchronized vagal activity generally mimics normal vagal traffic, which is sometimes reduced in these patients (who may, for example, suffer from heart failure or hypertension). Stable NSR typically results from the combined treatment modalities, thereby generally reducing the occurrence of AF.

Parameters of such rhythmic vagal stimulation typically include all or some of the following: (a) application of the stimulation as bursts synchronized with the patient's cardiac cycle, with each burst typically beginning at about 100 milliseconds after an R-wave, (b) about three pulses per burst (i.e., per cardiac cycle), (c) varying the number of pulses per burst responsive to sensed parameters of the patient's respiratory cycle or heart rate, and (d) varying the number of nerve fibers recruited responsive to sensed parameters of the patient's respiratory cycle or heart rate.

For example, vagal stimulation may be applied by cycling between a first set and a second set of parameters, applying each set for less than about 15 seconds, e.g., for between about 1 and about 4 seconds. The first set of parameters may include: (a) a low amplitude, e.g., 2 milliamps, so as to recruit a relatively small number of nerve fibers, (b) optional synchronization with inhalation, and (c) one pulse per trigger (PPT), for example applied at about 300 milliseconds after an R-wave. The second set of parameters may include: (a) a greater amplitude, e.g., 3 milliamps, so as to recruit a greater number of fibers, (b) optional synchronization with exhalation, and (c) three PPT, applied at about 300 milliseconds after an R-wave. Both sets of parameters optionally include a pulse width of about 1 millisecond and/or a pulse repetition interval of between about 4 and about 20 milliseconds.

In Combination with a Positive Inotropic Agent

A positive inotropic agent is administered for longer than one day, and vagal stimulation is applied using techniques described herein, using the typical stimulation parameters described hereinabove. Without the use of the vagal stimulation techniques described herein, drugs of this class (with the exception of digitalis) are generally administered only in an acute setting. In combination with vagal stimulation as described herein, however, the administration of the positive inotropic agent is hypothesized by the inventors to have the same or enhanced effect, without its chronotropic and proarrhythmic (ventricular) effects. In addition, it is hypothesized that in combination with vagal stimulation as described herein, the positive effects of the positive inotropic agent do not decline, or decline less, over time, when administered on a long-term basis.

For treating a stable patient, a positive inotropic agent is administered, and vagal stimulation is applied using parameters described hereinbelow for applying vagal stimulation with minimum heart rate reduction, or using the typical stimulation parameters described hereinabove. Without the use of the vagal stimulation techniques described herein, drugs of this class are generally not routinely used because of evidence indicating increased mortality mainly attributable to ventricular arrhythmia. Use of the vagal stimulation techniques described herein typically reduces the incidence of ventricular arrhythmia, thereby enabling the use of drugs of this class for longer-term treatment of stable patients.

### For Emergency Settings

In order to increase heart rate in an emergency setting (e.g., bradycardia and/or shock), atropine is administered, and vagal stimulation is applied, using the typical stimulation parameters described hereinabove, in order to increase heart rate and cardiac output.

In Combination with a Class IC Drug

A class IC drug is administered at a dosage greater than would normally be indicated or considered safe, and vagal stimulation is applied using parameters described hereinbelow for applying vagal stimulation with minimum heart rate reduction, or using the typical stimulation parameters described hereinabove, to counteract at least some of the side effects of the class IC drug.

In an embodiment of the present invention, stimulation, e.g., vagal stimulation, configured for inhibiting, delaying or preventing (i.e., delaying indefinitely) electrical remodeling in AF patients is applied in the absence of specific antiarrhythmic drug therapy. Such prevention of electrical remodeling alone is believed by the inventors to be therapeutically beneficial. For example, Takei M et al., in an article entitled, “Vagal stimulation prior to atrial rapid pacing protects the atrium from electrical remodeling in anesthetized dogs,” Jpn Circ J 65(12):1077-81 (2001), which is incorporated herein by reference, hypothesize, based on their experiments in anesthetized dogs, that vagal stimulation prior to atrial rapid pacing may protect the atrium from electrical remodeling.

In an embodiment of the present invention, a method for enhancing or sustaining the efficacy of a drug treatment for AF comprises administering a drug to the subject, applying signals to a parasympathetic site, such as the vagus nerve, and configuring the signals to reduce the mechanical tension on the atria. Such reduced mechanical tension generally reduces the risk of AF. For some applications, such stimulation is applied without administering the drug.

For some applications, such stimulation for the prevention of atrial remodeling (whether or not in conjunction with drug therapy) is applied generally constantly, using parameters described hereinbelow for applying stimulation with minimum heart rate reduction, or using the typical stimulation parameters described hereinabove. For other applications, such stimulation is only applied upon the detection of the occurrence of AF, such as by using one or more of the AF detection techniques described hereinabove.

In an embodiment of the present invention, control unit 32 configures the applied signals to have an antiarrhythmic effect on the atrium. Typical signal parameters in such a configuration include those described hereinbelow for applying stimulation with minimum heart rate reduction, or the typical stimulation parameters described hereinabove. The stimulation is typically applied to right vagus nerve 26, but may also be applied to left vagus nerve 25 or both vagus nerves together, or another of the parasympathetic sites listed hereinabove. For some applications, such antiarrhythmic stimulation is applied in conjunction with the rhythmic stimulation technique described hereinabove. For applications in which such antiarrhythmic stimulation is applied in combination with antiarrhythmic drug therapy, the combined treatment generally results in a synergistic effect.

In another embodiment of the present invention, the effectiveness of ventricular rate control drugs is typically enhanced by applying vagal stimulation in order to control the ventricular response rate. Such combined vagal stimulation and drug therapy generally results in a synergistic effect. Vagal stimulation techniques for controlling ventricular response rate may be used that are described in U.S. patent application Ser. No. 10/205,475, filed Jul. 24, 2002, entitled, “Selective nerve fiber stimulation for treating heart conditions,” which issued as U.S. Pat. No. 7,778,703, and is assigned to the assignee of the present patent application and is incorporated herein by reference, or by using other techniques known in the art.

In an embodiment of the present invention, the safety of a drug administered to subject 30 is improved by applying signals to vagus nerve 24 or another of the parasympathetic sites listed hereinabove, and configuring the signals so as to prevent adverse effects sometimes caused by the drug, such as repolarization abnormalities (e.g., prolongation of the QT interval), bradycardia, and/or ventricular tachyarrhythmia (e.g., ventricular fibrillation), such as using techniques described in the above-mentioned U.S. application Ser. No. 10/866,601.

In some cases, the drug can safely be administered to patients who otherwise could not tolerate the drug because of such adverse effects. (See, for example, Kwan H et al., “Cardiovascular adverse drug reactions during initiation of antiarrhythmic therapy for atrial fibrillation,” Can J Hosp Pharm 54:10-14 (2001), which is incorporated herein by reference, and which discusses the limitations side effects sometimes impose on drug success.) In addition, in some cases adverse effects of the drug are prevented or diminished by allowing the use of lower dosages of the drug (i.e., dosages lower than dosages determined independently of applying the vagal stimulation), by enhancing or sustaining the efficacy of the drug, as described hereinabove. For example, toxicity associated with digoxin may be prevented or reduced by enabling a lower dosage using these stimulation techniques.

Prolongation of the QT interval is an adverse effect sometimes caused by antiarrhythmic drugs. Vagal stimulation, using techniques described herein, typically shortens the QT interval, thereby offsetting the QT prolongation caused by such drugs. As a result, such drugs are generally safer, and, in some cases, more effective. In addition, such increased safety allows for the use of higher dosages of such drugs, if therapeutically indicated. For some applications, in order to obtain the QT interval reduction, and/or to prevent other side effects, such as abdominal pain, diarrhea, or ventricular arrhythmia not related to the QT interval, control unit 32 configures the signals applied to the vagus nerve using parameters described hereinbelow for applying vagal stimulation with minimum heart rate reduction.

Bradycardia is an adverse effect sometimes caused by antiarrhythmic drugs and heart rate control drugs. The use of a lower dosage of such drugs enabled by vagal stimulation techniques described herein generally reduces the likelihood of bradycardia, while obtaining a beneficial effect similar to that achieved at higher drugs dosages without such vagal stimulation. This vagal stimulation is typically applied using techniques described herein for minimizing reductions in heart rate as a result of the stimulation. In addition, in an embodiment, apparatus 20 monitors heart rate, such as by using ECG monitor 38, and, upon detection of bradycardia, activates pacemaker 42 to pace the heart. Alternatively or additionally, upon detection of bradycardia, apparatus 20 terminates or reduces the intensity of vagal stimulation.

Ventricular tachyarrhythmia is an adverse effect sometimes caused by antiarrhythmic drugs or positive inotropic drugs. Vagal stimulation, using techniques described herein, typically reduces or prevents tachyarrhythmia, premature ventricular contractions, ventricular tachycardia, accelerated idioventricular arrhythmia, and/or ventricular fibrillation, by reducing the propensity of cardiac tissue to spontaneously fire.

In an embodiment of the present invention, a method for enhancing or sustaining the efficacy of drug treatment for heart failure comprises administering a drug to patient 30 and applying signals to vagus nerve 24 that innervates heart 28 of the patient. The signals are configured so as to treat the heart failure, which typically results in a synergistic effect between the therapeutic benefit of the drug and the vagal stimulation. For example, the drug may include positive inotropic drugs such as digoxin, dopamine, dobutamine, adrenaline, aminone, or milrinone.

Alternatively or additionally, the signals are configured so as to prevent adverse effects sometimes caused by the heart failure drug, such as ventricular arrhythmia and/or ventricular tachycardia. For some applications, ventricular tachycardia is prevented using techniques described hereinabove for controlling ventricular response rate using vagal stimulation. For some applications, arrhythmia is prevented by elevation of vagal tone and application of rhythmic synchronized vagal stimulation, for example using the parameters for rhythmic vagal stimulation described hereinabove.

In addition, in some cases adverse effects of the heart failure drug are prevented or diminished by allowing the use of lower dosages of the drug because of the synergistic effect of the vagal stimulation with the drug treatment.

In an embodiment of the present invention, a method for enhancing or sustaining the efficacy of antithrombotic therapy comprises administering an antithrombotic drug to patient 30 and applying signals to vagus nerve 24 that innervates heart 28 of the patient. The signals are configured so as to increase atrial motion, which typically results in a synergistic effect between the therapeutic benefit of the drug and the vagal stimulation. Such vagal stimulation thus may (a) increase the efficacy of the antithrombotic drug, and/or (b) allow the use of a lower dosage of the drug, without reducing the efficacy of the drug. As used in the present patent application including the claims, antithrombotic drugs are to be understood as drugs that are intended to reduce the risk of thromboembolic events, including, but not limited to, anticoagulation drugs that inhibit the coagulation cascade (e.g., warfarin, heparin, low molecular weight heparin (LMWH)), and drugs that inhibit platelet aggregation (e.g., aspirin and clopidogrel). Increased efficacy caused by vagal stimulation may increase the effectiveness of a platelet aggregation inhibition drug, thereby allowing the use of such a drug instead of anticoagulation drugs, which typically have greater side effects and risks, and require more precise dosaging, than platelet aggregation inhibition drugs. In addition, use of a lower dosage may reduce complications associated with typical dosages of antithrombotic drugs. For antithrombotic drug regimens in which dosages are selected to achieve a target international normalized ratio (INR) of 2.5, the synergistic effect of the vagal stimulation with the drug treatment may allow the same beneficial effect to be achieved at a lower INR, e.g., 1.5, thereby reducing drug complications. For some applications, antithrombotic therapy is enhanced or sustained by elevation of vagal tone and application of rhythmic synchronized vagal stimulation, for example using the parameters for rhythmic vagal stimulation described hereinabove.

In an embodiment of the present invention, stimulation is applied and configured to prevent atrial electrical remodeling caused by heart failure (see Li D et al., “Promotion of Atrial Fibrillation by Heart Failure in Dogs: Atrial Remodeling of a Different Sort,” Circulation 100(1):87-95 (1999), which is incorporated herein by reference). For some applications, such stimulation is applied to increase the efficacy and/or safety of a heart failure drug; for other applications, such stimulation is applied in the absence of specific drug therapy. Such prevention of electrical remodeling alone is believed by the inventors to be therapeutically beneficial. In an embodiment, stimulation is applied and configured to treat a subject suffering from both AF and heart failure, such as by preventing atrial electrical remodeling, and/or by increasing the efficacy and/or safety of one or more drugs for AF and/or heart failure.

In an embodiment of the present invention, a method for enhancing the efficacy of drug treatment for heart failure comprises administering a “preload reduction” drug, such as an ACE inhibitor, nitrate, or sodium nitroprusside, to patient 30, and applying signals to vagus nerve 24 that innervates heart 28 of the patient. Such preload reduction drugs are intended to reduce the pressure in the venous system. During heart failure, atrial contraction sometimes pushes blood back into the venous and pulmonary systems. To minimize this unwanted effect, the signals applied to the vagus nerve are configured so as to decrease atrial contractile force, using the typical stimulation parameters described hereinabove, for example with a short “on” time (e.g., between about 1 and about 15 seconds) and a longer “off” time (e.g., between about 5 and about 20 seconds). For some applications, the “on” and “off” times are equal, and for other applications, the “off” time is longer than the “on” time. In an embodiment, this vagal stimulation treatment is applied without the preload reduction drug treatment.

In an embodiment of the present invention, a method for increasing vagal tone comprises applying signals to vagus nerve 24 or another of the parasympathetic sites listed hereinabove, and configuring the signals to deliver parasympathetic nerve stimulation to heart 28, while at the same time minimizing the heart-rate-lowering effects of the stimulation. Such treatment generally results in the beneficial effects of vagal or other parasympathetic stimulation that are not necessarily dependent on the heart-rate reduction effects of such stimulation. (See, for example, Vanoli E et al., “Vagal stimulation and prevention of sudden death in conscious dogs with a healed myocardial infarction,” Circ Res 68(5):1471-81 (1991), which is incorporated herein by reference.) Therefore, such stimulation is generally useful for treating conditions such as AF, heart failure, atherosclerosis, restenosis, myocarditis, cardiomyopathy, post-myocardial infarct remodeling, and hypertension. In addition, such treatment is believed by the inventors to reduce the risk of sudden cardiac death in some subjects (such as those with hypertrophic cardiomyopathy or congenital long QT syndrome). Furthermore, such treatment is believed by the inventors to be beneficial for the treatment of some non-cardiovascular conditions, such as an autoimmune disease, an autoimmune inflammatory disease, multiple sclerosis, encephalitis, myelitis, immune-mediated neuropathy, myositis, dermatomyositis, polymyositis, inclusion body myositis, inflammatory demyelinating polyradiculoneuropathy, Guillain Barre syndrome, myasthenia gravis, inflammation of the nervous system, SLE (systemic lupus erythematosus), rheumatoid arthritis, vasculitis, polyarteritis nodosa, Sjogren syndrome, mixed connective tissue disease, glomerulonephritis, thyroid autoimmune disease, sepsis, meningitis, a bacterial infection, a viral infection, a fungal infection, sarcoidosis, hepatitis, and portal vein hypertension, obesity, constipation, irritable bowl syndrome, rheumatoid arthritis, glomerulonephritis, hepatitis, pancreatitis, thyroiditis, type I diabetes, and type II diabetes. For some applications, conditions mentioned in this paragraph are treated by applying vagal stimulation, and not necessarily minimizing the heart-rate-lowering effects of the stimulation.

Such parasympathetic stimulation is also beneficial for treating some conditions or under some circumstances in which heart rate reduction is not indicated or is contraindicated. For example, such parasympathetic stimulation is typically appropriate:
for treating heart failure subjects that suffer from bradycardia when taking beta-blockers;
at nighttime, when heart rate is naturally lower;
during exercise, such as when the heart rate is already within a desired range and further decreases may reduce exercise tolerance;
for subjects receiving heart-rate lowering drugs, who have achieved a heart rate within a desired range prior to beginning stimulation, and therefore would not benefit from further heart rate reduction;
for subjects suffering from low cardiac output, for whom heart rate reduction may further reduce cardiac output;
during acute myocardial infarction with cardiogenic shock;
for subjects who experience discomfort or a reduction in exercise capacity when the heart rate is reduced; and
for subjects having a tendency towards bradycardia when receiving vagal or parasympathetic stimulation.

In an embodiment of the present invention, in order to increase vagal tone while at the same time minimizing or preventing the heart-rate-lowering effects of the stimulation, control unit 32 applies the signals to the parasympathetic site as a burst of pulses during each cardiac cycle, with one or more of the following parameters:
Timing of the stimulation: delivery of the burst of pulses begins after a variable delay following each P-wave, the length of the delay equal to between about two-thirds and about 90% of the length of the subject's cardiac cycle. Such a delay is typically calculated on a real-time basis by continuously measuring the length of the subject's cardiac cycle.
Pulse duration: each pulse typically has a duration of between about 200 microseconds and about 2.5 milliseconds for some applications, or, for other applications, between about 2.5 milliseconds and about 5 milliseconds.
Pulse amplitude: the pulses are typically applied with an amplitude of between about 0.5 and about 5 mA, e.g., about 1 mA.
Pulse repetition interval (PRI): the pulses within the burst of pulses typically have a PRI (the time from the initiation of a pulse to the initiation of the following pulse) of, on average, at least 20 ms, such as at least 30 ms, e.g., at least 50 ms or at least 75 ms; alternatively, the PRI may be between about 2 and about 10 milliseconds, e.g., about 2.5 milliseconds.
Pulse period: the burst of pulses typically has a total duration of between about 0.2 and about 40 milliseconds, e.g., about 1 millisecond.
Pulses per trigger (PPT): the burst of pulses typically contains between about 1 and about 10 pulses, e.g., about 2 pulses.
Site: for some applications, the left vagus nerve is stimulated in order to minimize the heart-rate-lowering effects of vagal stimulation.
Duty cycle: stimulation is typically applied only once every several heartbeats, or once per heartbeat, when a stronger effect is desired.
On/off status: for some applications, stimulation is always “on”, i.e., constantly applied (in which case, parameters closer to the lower ends of the ranges above are typically used). For other applications, on/off cycles vary between a few seconds to several dozens of seconds, e.g., “on” for about 36 seconds, “off” for about 120 seconds, “on” for about 3 seconds, “off” for about 9 seconds.

For example, stimulation may be applied to a subject having a heart rate of 60 BPM, with the intention of minimally reducing the subject's heart rate. The burst of pulses may be delivered beginning about 750 milliseconds after each R-wave of the subject. The stimulation may be applied with one pulse per trigger (PPT), and having an amplitude of 1 mA. The stimulation may be cycled between “on” and “off” periods, with each “on” period having a duration of about two seconds, i.e., two heart beats, and each “off” period having a duration of about 4 seconds.

In an embodiment of the present invention, control unit 32 is configured to sense a heart rate of the subject, and to apply the stimulation with minimal-heart-rate-reducing parameters only when the sensed heart rate is below a threshold rate. For some applications, the threshold is a normal heart rate for the subject, or a percentage of the normal heart rate, e.g., between about 80% and about 100%, such as between about 80% and about 95%, or between about 80% and about 120%, e.g., between about 95% and about 105%, such as about 100%. The normal heart rate of the subject may be sensed by control unit 32, or entered into the control unit by a medical professional. Alternatively, the threshold is a normal heart rate for typical subjects, such as between about 50 and about 80 BPM, or a percentage of the normal heart rate, e.g., between about 80% and about 100%, such as between about 80% and about 95%, or between about 80% and about 120%, e.g., between about 95% and about 105%, such as about 100%. Applying the stimulation only when the sensed heart rate is below the threshold rate further reduces any heart-rate-lowering effects of the stimulation, because the stimulation has less effect on heart rate at lower heart rates. Furthermore, it is sometimes undesirable to apply the stimulation when the subject's heart rate is elevated, either because of normal causes, such as exercise, or because of pathological causes, such as ventricular or atrial tachycardia.

Alternatively or additionally, the control unit drives pacemaker 42 to pace the heart, so as to prevent any heart-rate lowering effects of stimulation. Typically, the control unit paces the heart at a rate that is similar to the rate when the device is in “off” mode. Control unit 32 then applies the stimulation, typically using the typical stimulation parameters described hereinabove. This stimulation generally does not lower the heart rate, because of the pacemaker pacing. For some applications, control unit 32 applies the signals, and senses the heart rate after applying the signals. The control unit drives pacemaker 42 to pace the heart if the sensed heart rate falls below a threshold heart rate. The threshold heart rate is typically equal to a heart rate of the subject prior to commencing the stimulation, for example, as sensed by control unit 32. The control unit thus typically maintains the heart rate at a rate above a bradycardia threshold rate, unlike conventional pacemakers which are typically configured to pace the heart only when the rate falls below a bradycardia threshold rate. Upon termination of stimulation, control unit 32 typically drives pacemaker 42 to continue pacing the heart for a period typically having a duration between about 0 and about 30 seconds, such as about 5 seconds.

In an embodiment of the present invention, control unit 32 drives pacemaker 42 to pace the heart, and configures the signals applied to the vagal or other parasympathetic site using the typical stimulation parameters described hereinabove. For some applications, the higher ends of the ranges of values for one or more of these parameters are applied. The use of the pacemaker generally prevents any heart-rate-lowering effects of such stimulation.

In an embodiment of the present invention, control unit 32 applies minimal-heart-rate-lowering stimulation using a feedback loop. The control unit calculates an average heart rate (ventricular and/or atrial rate) of the subject. The control unit then applies signals to vagus nerve 24 or another of the parasympathetic sites listed hereinabove, using the minimal heart rate reduction parameters described hereinabove. During such stimulation, the control unit substantially continuously monitors the resulting heart rate. If the heart rate declines by more than a certain percentage (e.g., by more than about 5%, such as from 100 BPM to 90 BPM), the control unit adjusts the stimulation parameters in order to further minimize the heart-rate-lowering effect of the stimulation. For example, the control unit may adjust the stimulation parameters by reducing the amplitude of the stimulation, changing the timing of the stimulation, reducing the frequency of the stimulation, reducing the duration of each pulse, and/or reducing the duration of the stimulation period.

In an embodiment of the present invention, a method for preventing or reducing fibrosis and/or inflammation of the heart comprises configuring control unit 32 to apply signals to vagus nerve 24 that innervates heart 28 of the patient. Substantially continuous application of such stimulation generally modulates immune system responses, thereby reducing atrial, ventricular, and/or coronary inflammation and/or fibrosis. Such stimulation is typically applied using the typical stimulation parameters described hereinabove, or the parameters described hereinabove for minimal heart rate reduction. For some applications, such stimulation is applied for more than about three weeks. Conditions that are believed to be at least partially immune-modulated, and therefore to generally benefit from such vagal stimulation, include, but are not limited to, atrial and ventricular remodeling (e.g., induced by AF, heart failure, myocarditis, and/or myocardial infarct), restenosis, and atherosclerosis.

In an embodiment of the present invention, control unit 32 is configured to apply signals to vagus nerve 24 of subject 30 or another of the parasympathetic sites listed hereinabove, and to configure the signals to inhibit propagation of naturally-generated efferent action potentials in the vagus nerve. Typically, the signals are additionally configured to inhibit no more than about 10% of naturally-generated afferent action potentials traveling through the vagus nerve. It is hypothesized by the inventors that such inhibition is useful for treating AF, typically by enhancing drug efficacy, and for preventing bradycardia.

In an embodiment of the present invention, electrical signals are applied by electrode device 22, typically on a long-term basis, to vagus nerve 24 of a subject not necessarily suffering from a heart condition, in order to increase the life expectancy, quality of life, and/or healthiness of the subject. Such signals are typically configured to not reduce the heart rate below normal range for a typical human. Typical parameters of such stimulation include those described hereinabove for minimal heart-rate-reducing stimulation, for periods during which the heart rate is at a desired level, and those described hereinabove for lowering heart rate, when it is desired to lower the heart rate from above normal to normal. For some applications, a determination regarding whether to attempt to lower the heart rate is made responsive to physiological parameters sensed using a sensor, such as an activity sensor, a respiration sensor, or accelerometer 39. Such chronic vagal stimulation is hypothesized by the inventors to be effective for increasing life expectancy, quality of life, and/or healthiness by (a) causing a reduction in cardiovascular disease and/or events, (b) having an anti-inflammatory effect, (c) reducing heart rate from faster than desirable to desirable normal rates, (d) reducing metabolic rate, and/or (e) generally having a calming and relaxing effect.

In an embodiment of the present invention, apparatus 20 is adapted to be used prior to, during, and/or following a clinical procedure. In addition to configuring the stimulation to reduce the likelihood of the occurrence of an episode of AF, for some applications control unit 32 configures the current to reduce a potential immune-mediated response to the procedure. Such a reduction generally promotes healing after the procedure. (See Borovikova L V et al., “Vagus nerve stimulation attenuates the systemic inflammatory response to endotoxin,” Nature 405(6785):458-62 (2000), which is incorporated herein by reference, and which describe an anti-inflammatory cholinergic pathway that may mediate this reduction in immune-related response.) When the procedure is heart-related, the stimulation additionally typically reduces mechanical stress by lowering heart rate and pressures, reduces heart rate, and/or improves coronary blood flow.

For some applications, the stimulation commences after the conclusion of the procedure. For some applications, the stimulation commences prior to the commencement of the procedure. Alternatively, the stimulation commences during the procedure. Further alternatively, the stimulation is applied before and after the procedure, but not during the procedure.

For some applications, the clinical procedure is selected from one of the following:
coronary artery bypass graft (CABG) surgery. In addition to the benefits of stimulation described above, vagal tone was shown by Cumming J E et al. to be effective in reducing the likelihood of postoperative atrial fibrillation (AF), increasing the likelihood that the graft will stay in place, reducing the likelihood of graft failure (e.g., via stenosis), improving healing from the surgery, and/or reducing pain associated with the surgery. It is hypothesized by the inventors that such a reduction in the likelihood of postoperative AF is due, at least in part, to the mechanical stress reduction and rhythmic vagal activity promoted by vagal or other parasympathetic stimulation. For some applications, the stimulation is applied for between 1 and 7 days after the CABG surgery, intermittently or continuously.
valve replacement surgery. In addition to the benefits of stimulation described above, stimulation generally reduces the likelihood of postoperative AF, promotes healing of the heart, and reduces the likelihood of other conductance abnormalities.
heart transplantation. In addition to the benefits of stimulation described above, stimulation generally reduces the likelihood of rejection of the transplanted heart. For some applications, stimulation is applied on a short-term basis, e.g., for less than about 7 days before and/or 7 days after the heart transplantation. Alternatively, stimulation is applied long-term, e.g., for more than about 2 weeks before and/or 2 weeks after the procedure.
percutaneous transluminal coronary angioplasty (PTCA) and/or stenting procedures. In addition to the benefits of stimulation described above, stimulation generally reduces the likelihood of restenosis, which is believed to be at least in part immune-mediated. In addition, stimulation induces coronary dilation, which generally reduces the likelihood of restenosis.
carotid endarterectomy. In addition to the benefits of stimulation described above, stimulation generally reduces the likelihood of restenosis, which is believed to be at least in part immune-mediated.
other bypass surgery. In addition to the benefits of stimulation described above, stimulation generally reduces the likelihood of restenosis in the grafted bypass (natural or artificial).

In an embodiment of the present invention, control unit 32 is configured to operate in one of the following modes:
stimulation is applied using fixed programmable parameters, i.e., not in response to any feedback, target heart rate, or target heart rate range. These parameters may be externally updated from time to time, for example by a physician;
stimulation is not applied when the heart rate of the subject is lower than the low end of the normal range of a heart rate of the subject and/or of a typical human subject;
stimulation is not applied when the heart rate of the subject is lower than a threshold value equal to the current low end of the range of the heart rate of the subject, i.e., the threshold value is variable over time as the low end generally decreases as a result of chronic stimulation treatment;
stimulation is applied only when the heart rate of the subject is within the normal of range of a heart rate of the subject and/or of a typical human subjects; or
stimulation is applied only when the heart rate of the subject is greater than a programmable threshold value, such as a rate higher than a normal rate of the subject and/or a normal rate of a typical human subject. This mode generally removes peaks in heart rate.

For many of the applications of parasympathetic stimulation described herein, electrode device 22 typically comprises one or more electrodes, such as monopolar, bipolar or tripolar electrodes. Electrode device 22 is typically placed: (a) around vagus nerve 24, (b) around vagus nerve 24 and the carotid artery (configuration not shown), or (c) inside the carotid artery in a position suitable for vagal stimulation (not shown). Depending on the particular application, one or more electrode devices 22 may be positioned to stimulate the left or right vagus nerve, either above or below the cardiac branch bifurcation. For some applications, the electrodes comprise cuff electrodes, ring electrodes, and/or point electrodes. Typically, the electrodes stimulate the nerve without coming in direct contact therewith, by applying an electrical field to the nerve. Alternatively, the electrodes stimulate the nerve by coming in direct contact therewith. For applications in which excitatory signals are applied to vagus nerve 24 (as opposed to inhibiting signals), control, control unit 32 typically configures the signals to induce the propagation of efferent nerve impulses towards heart 28.

In some embodiments of the present invention, when configuring vagal stimulation to induce the propagation of efferent nerve impulses towards heart 28, control unit 32 drives electrode device 22 to (a) apply signals to induce the propagation of efferent nerve impulses towards heart 28, and (b) suppress artificially-induced afferent nerve impulses towards a brain 35 of the subject (FIG. 1), in order to minimize unintended side effects of the signal application.

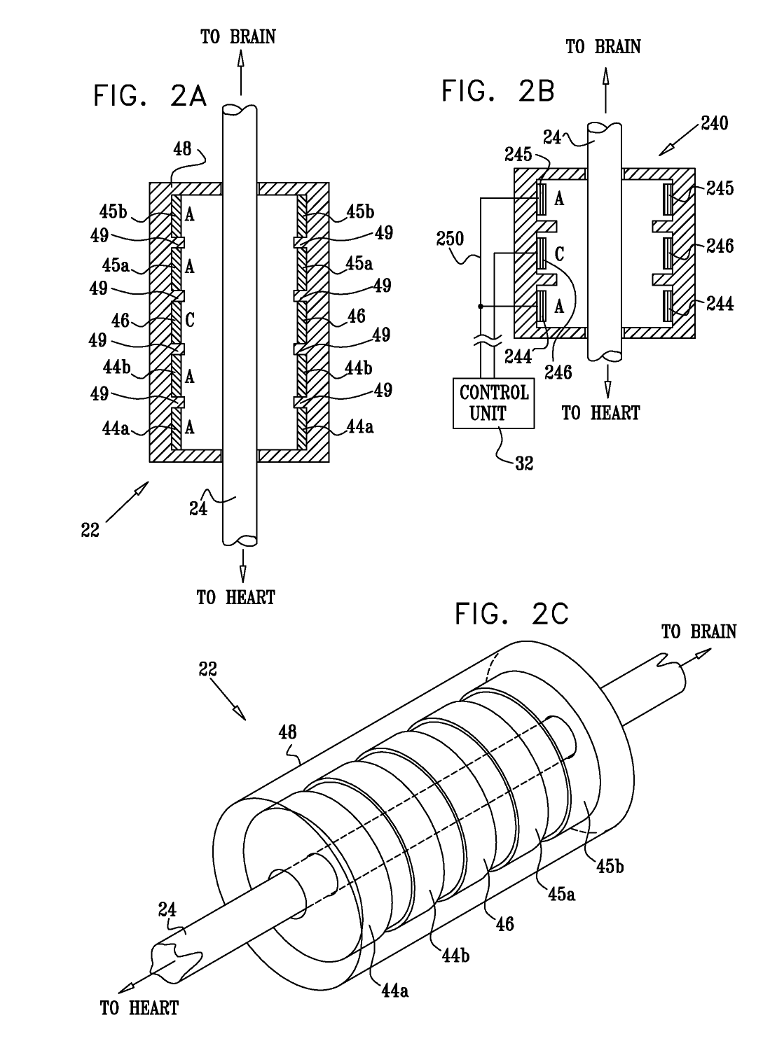

FIG. 2A is a simplified cross-sectional illustration of a generally-cylindrical electrode device 22 applied to vagus nerve 24, in accordance with an embodiment of the present invention. Electrode device 22 comprises a central cathode 46 for applying a negative current (“cathodic current”) in order to stimulate vagus nerve 24, as described below. Electrode device 22 additionally comprises a set of one or more anodes 44 (44a, 44b, herein: “efferent anode set 44”), placed between cathode 46 and the edge of electrode device 22 closer to heart 28 (the “efferent edge”). Efferent anode set 44 applies a positive current (“efferent anodal current”) to vagus nerve 24, for blocking action potential conduction in vagus nerve 24 induced by the cathodic current, as described below. Typically, electrode device 22 comprises an additional set of one or more anodes 45 (45a, 45b, herein: “afferent anode set 45”), placed between cathode 46 and the edge of electrode device 22 closer to brain 35. Afferent anode set 45 applies a positive current (“afferent anodal current”) to vagus nerve 24, in order to block propagation of action potentials in the direction of the brain during application of the cathodic current.

For some applications, the one or more anodes of efferent anode set 44 are directly electrically coupled to the one or more anodes of afferent anode set 45, such as by a common wire or shorted wires providing current to both anode sets, substantially without any intermediary elements. Typically, coatings on the anodes, shapes of the anodes, positions of the anodes, the sizes of the anodes and/or distances of the various anodes from the nerve are regulated so as to produce desired ratios of currents delivered through the various anodes, and/or desired activation functions delivered through or caused by the various anodes. For example, by varying one or more of these characteristics, the relative impedance between the respective anodes and central cathode 46 is regulated, whereupon more anodal current is driven through the one or more anodes having lower relative impedance. In these applications, central cathode 46 is typically placed closer to one of the anode sets than to the other, for example, so as to induce asymmetric stimulation (i.e., not necessarily unidirectional in all fibers) between the two sides of the electrode device. The closer anode set typically induces a stronger blockade of the cathodic stimulation.

Reference is now made to FIG. 2B, which is a simplified cross-sectional illustration of a generally-cylindrical electrode device 240 applied to vagus nerve 24, in accordance with an embodiment of the present invention. Electrode device 240 comprises exactly one efferent anode 244 and exactly one afferent anode 245, which are electrically coupled to each other, such as by a common wire 250 or shorted wires providing current to both anodes 244 and 245, substantially without any intermediary elements. (For some applications, electrode device 240 comprises more than one efferent anode 244 and/or more than one afferent anode 245.) The cathodic current is applied by a cathode 246 with an amplitude sufficient to induce action potentials in large- and medium-diameter fibers (e.g., A- and B-fibers), but insufficient to induce action potentials in small-diameter fibers (e.g., C-fibers).

Reference is again made to FIG. 2A. Cathode 46 and anode sets 44 and 45 (collectively, “electrodes”) are typically mounted in a housing such as an electrically-insulating cuff 48 and separated from one another by insulating elements such as protrusions 49 of the cuff. Typically, the width of the electrodes is between about 0.5 and about 2 millimeters, or is equal to approximately one-half the radius of the vagus nerve. The electrodes are typically recessed so as not to come in direct contact with vagus nerve 24. For some applications, such recessing enables the electrodes to achieve generally uniform field distributions of the generated currents and/or generally uniform values of the activation function defined by the electric potential field in the vicinity of vagus nerve 24. Alternatively or additionally, protrusions 49 allow vagus nerve 24 to swell into the canals defined by the protrusions, while still holding the vagus nerve centered within cuff 48 and maintaining a rigid electrode geometry. For some applications, cuff 48 comprises additional recesses separated by protrusions, which recesses do not contain active electrodes. Such additional recesses accommodate swelling of vagus nerve 24 without increasing the contact area between the vagus nerve and the electrodes. For some applications, the distance between the electrodes and the axis of the vagus nerve is between about 1 and about 4 millimeters, and is greater than the closest distance from the ends of the protrusions to the axis of the vagus nerve. Typically, protrusions 49 are relatively short (as shown). The distance between the ends of protrusions 49 and the center of the vagus nerve is typically between about 1 and 3 millimeters. (Generally, the diameter of the vagus nerve is between about 2 and 3 millimeters.) Alternatively, for some applications, protrusions 49 are longer and/or the electrodes are placed closer to the vagus nerve in order to reduce the energy consumption of electrode device 22.

In an embodiment of the present invention, efferent anode set 44 comprises a plurality of anodes 44, typically two anodes 44a and 44b, spaced approximately 0.5 to 2.0 millimeters apart. Application of the efferent anodal current in appropriate ratios from the plurality of anodes generally minimizes the “virtual cathode effect,” whereby application of too large an anodal current stimulates rather than blocks fibers. In an embodiment, anode 44a applies a current with an amplitude equal to about 0.5 to about 5 mA (typically one-third of the amplitude of the current applied by anode 44b). When such techniques are not used, the virtual cathode effect generally hinders blocking of smaller-diameter fibers, as described below, because a relatively large anodal current is generally necessary to block such fibers.

Anode 44a is typically positioned in cuff 48 to apply current at the location on vagus nerve 24 where the virtual cathode effect is maximally generated by anode 44b. For applications in which the blocking current through anode 44b is expected to vary substantially, efferent anode set 44 typically comprises a plurality of virtual-cathode-inhibiting anodes 44a, one or more of which is activated at any time based on the expected magnitude and location of the virtual cathode effect.

Likewise, afferent anode set 45 typically comprises a plurality of anodes 45, typically two anodes 45a and 45b, in order to minimize the virtual cathode effect in the direction of the brain. In certain electrode configurations, cathode 46 comprises a plurality of cathodes in order to minimize the “virtual anode effect,” which is analogous to the virtual cathode effect.

FIG. 2C is a simplified perspective illustration of electrode device 22, in accordance with an embodiment of the present invention. When applied to vagus nerve 24, electrode device 22 typically encompasses the nerve. As described, control unit 32 typically drives electrode device 22 to (a) apply signals to vagus nerve 24 in order to induce the propagation of efferent action potentials towards heart 28, and (b) suppress artificially-induced afferent action potentials towards brain 35. The electrodes typically comprise ring electrodes adapted to apply a generally uniform current around the circumference of the nerve, as best shown in FIG. 2C.

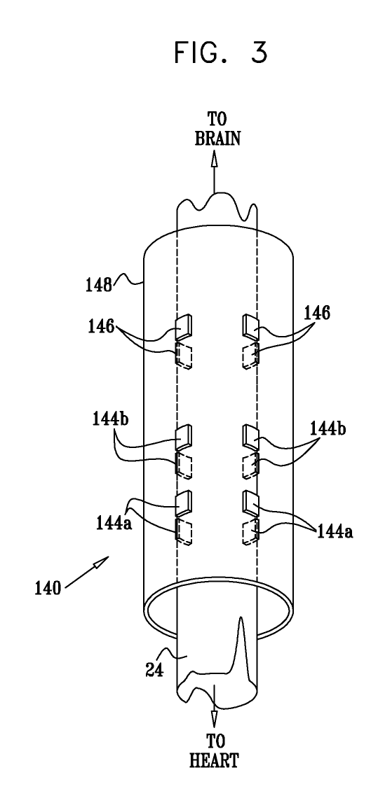

FIG. 3 is a simplified perspective illustration of a multipolar point electrode device 140 applied to vagus nerve 24, in accordance with an embodiment of the present invention. In this embodiment, anodes 144a and 144b and a cathode 146 typically comprise point electrodes (typically 2 to 100), fixed inside an insulating cuff 148 and arranged around vagus nerve 24 so as to selectively stimulate nerve fibers according to their positions inside the nerve. In this case, techniques may be used that are described in the following articles, all of which are incorporated herein by reference: (a) Grill WM et al., “Inversion of the current-distance relationship by transient depolarization,” IEEE Trans Biomed Eng, 44(1):1-9 (1997); (b) Goodall E V et al., “Position-selective activation of peripheral nerve fibers with a cuff electrode,” IEEE Trans Biomed Eng, 43(8):851-6 (1996); and/or (c) Veraart C et al., “Selective control of muscle activation with a multipolar nerve cuff electrode,” IEEE Trans Biomed Eng, 40(7):640-53 (1993). The point electrodes typically have a surface area between about 0.01 mm2 and 1 mm2. In some applications, the point electrodes are in contact with vagus nerve 24, as shown, while in other applications the point electrodes are recessed in cuff 148, so as not to come in direct contact with vagus nerve 24, similar to the recessed ring electrode arrangement described above with reference to FIG. 2A. For some applications, one or more of the electrodes, such as cathode 146 or anode 144a, comprise a ring electrode, as described with reference to FIG. 2C, such that electrode device 140 comprises both ring electrode(s) and point electrodes (configuration not shown). Additionally, electrode device 22 optionally comprises an afferent anode set (positioned like anodes 45a and 45b in FIG. 2A), the anodes of which comprise point electrodes and/or ring electrodes.

Alternatively, ordinary, non-cuff electrodes are used, such as when the electrodes are placed on the epicardial fat pads instead of on the vagus nerve.

In an embodiment of the present invention, a method for surgically implanting electrode device 22 comprises: (a) placing the electrode device around vagus nerve 24, (b) during the implantation procedure, introducing saline solution into the electrode device such that the solution is in contact with both the electrodes and the nerve, and (c) measuring an inter-electrode impedance during the implantation procedure. Such an impedance measurement enables the surgeon to determine during the procedure (a) whether the electrodes are positioned appropriately, (b) whether sufficient saline solution has been introduced into and remained in electrode device 22, (c) whether the electrodes are the correct size for the nerve, and (d) whether the electrodes are in good contact with the nerve. Expected values for the impedance measurement, and their typical interpretations, include:
a low value, such as between about 100 and about 300 ohms, which typically occurs if the electrodes are in poor contact with the nerve, such as because the diameter of the electrode is larger than that of the nerve. When there is such poor contact, the electrodes are short-circuited by the saline solution, resulting in the low impedance;
a high value, such as greater than about 1000 ohms, which typically occurs if electrode device 22 is not filled properly with saline solution, which causes a disconnect between the electrodes and the nerve; or
a medium value, such as between about 300 and about 1000 ohms, which indicates that the electrodes are in good contact with the nerve, so that most of the current travels through the nerve.

If the impedance differs from an expected value, the surgeon corrects the placement by, for example, repositioning the electrode device, removing the electrode device and implanting another electrode device having a different size, and/or introducing additional saline solution into the electrode device. The techniques of this embodiment are also applicable to implanting electrode devices on a body tissue other than the vagus nerve.

For some applications, a quality-control method for screening electrode devices 22 during the manufacture thereof comprises (a) placing each of the electrode devices in saline solution and introducing saline solution into the electrode device such that the saline solution is in contact with the electrodes thereof, (b) measuring an inter-electrode impedance, and (c) discarding the electrode device if the measured impedance is outside of a tolerance range, which range typically has a low end of between 100 and 300 ohms, such as 200 ohms, and a high end of between 1000 and 2000 ohms, such as 2000 ohms.

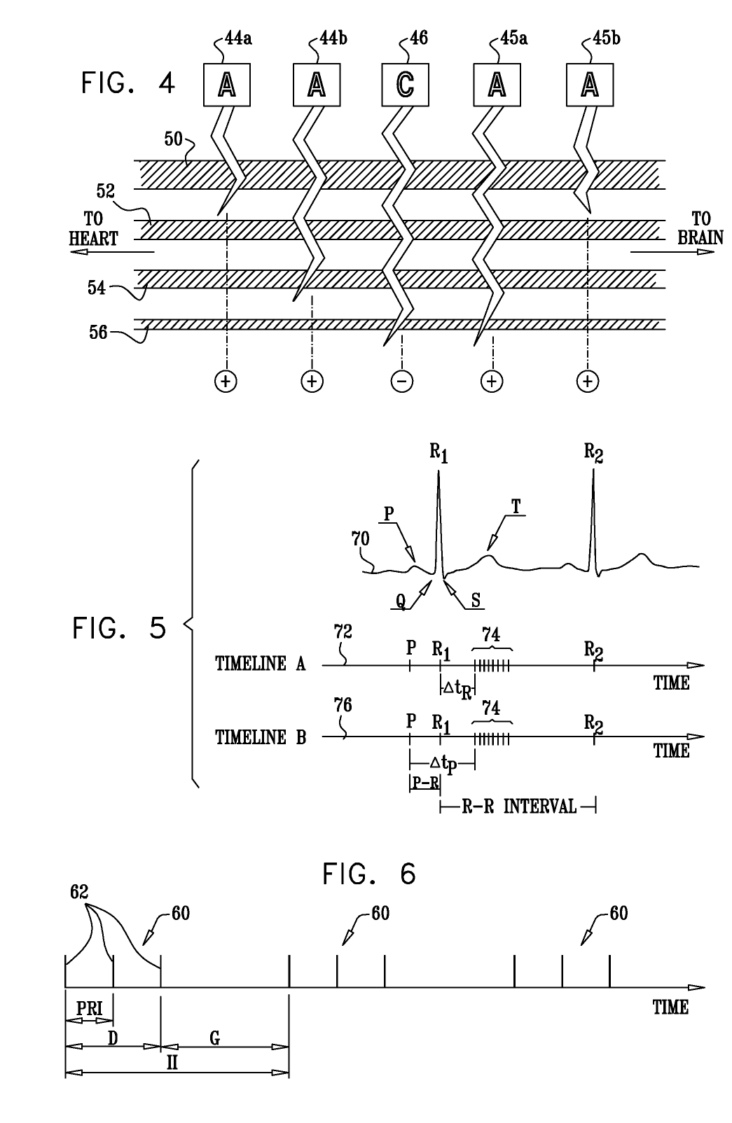

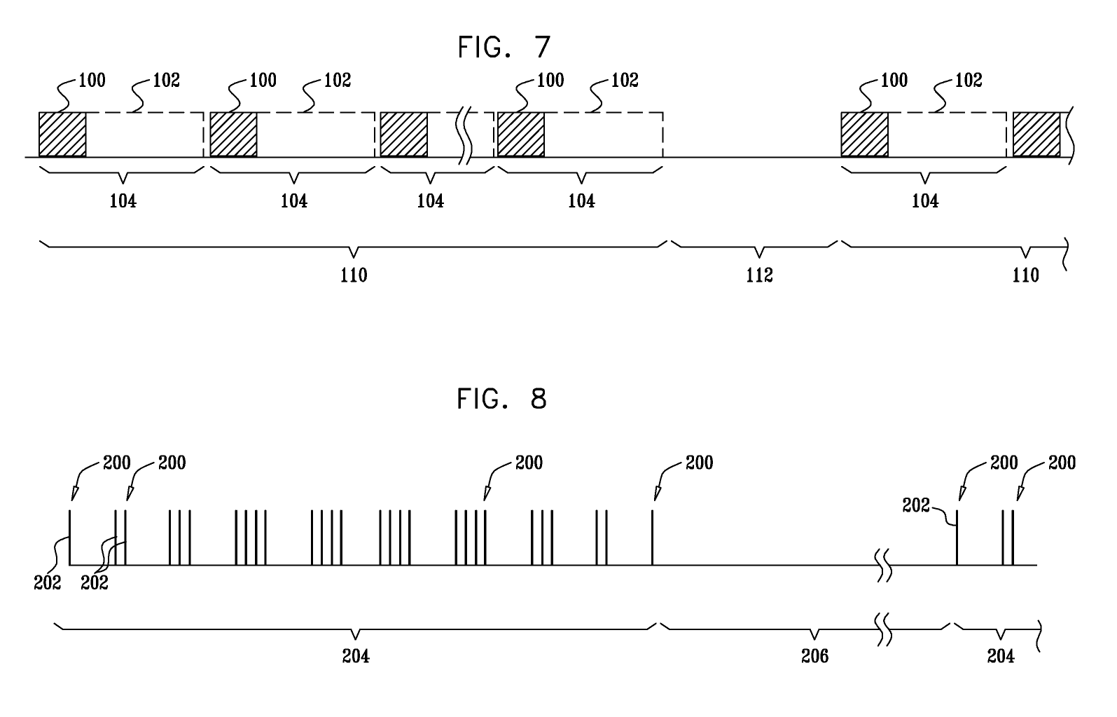

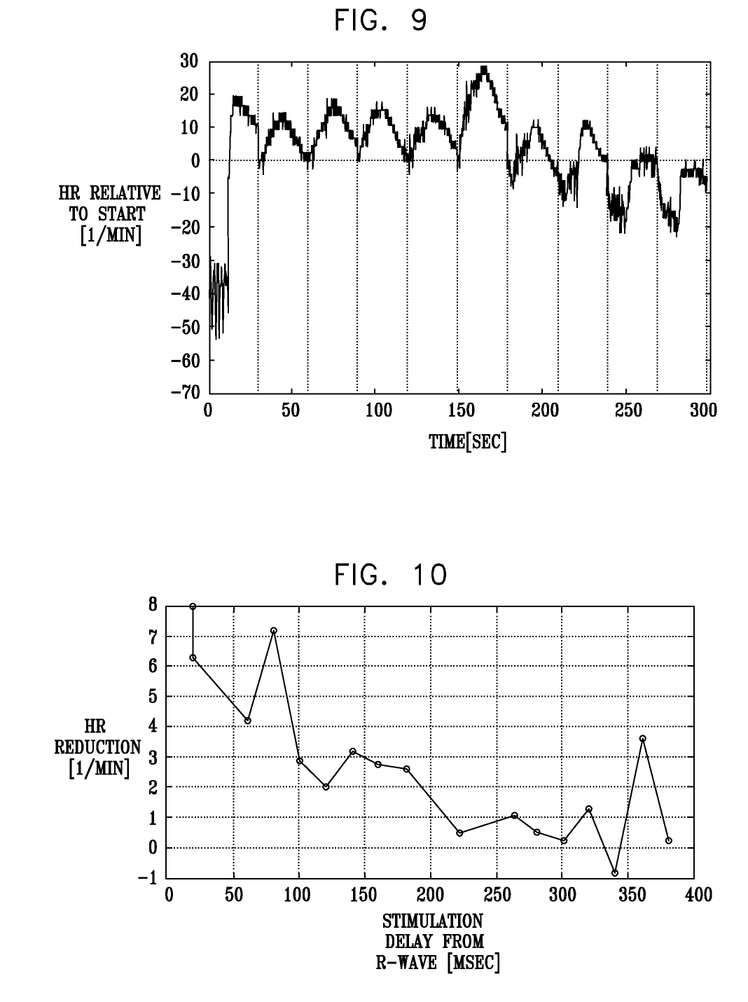

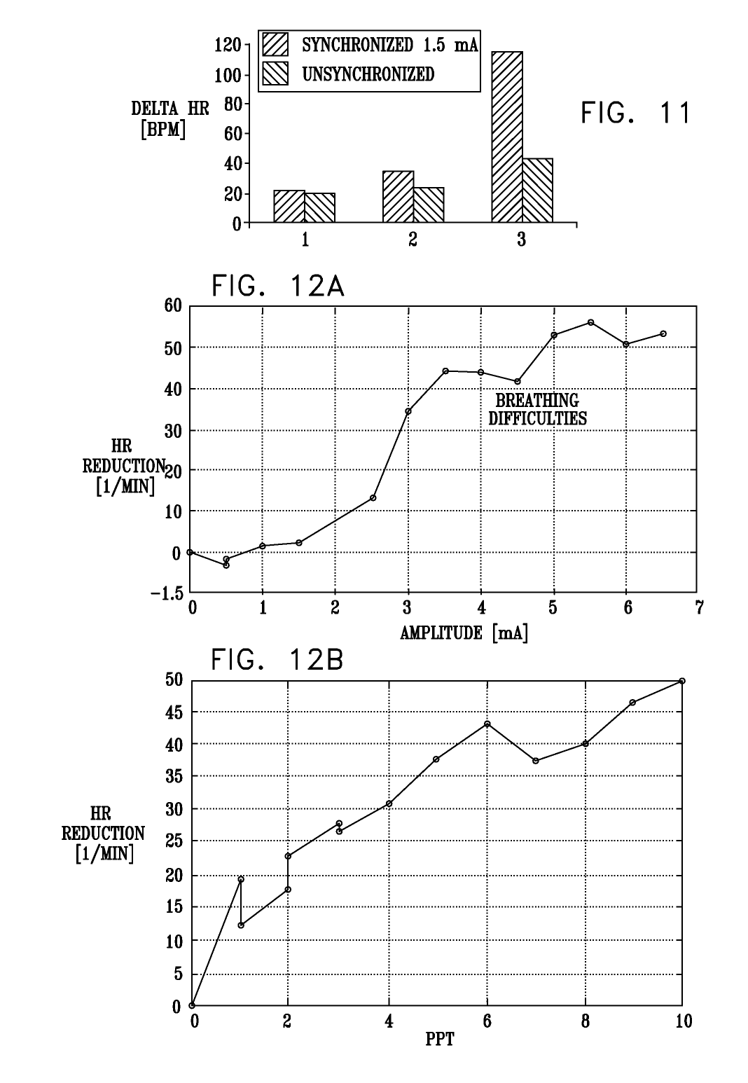

FIG. 4 is a conceptual illustration of the application of current to vagus nerve 24 in order to achieve smaller-to-larger diameter fiber recruitment, in accordance with an embodiment of the present invention. When inducing efferent action potentials towards heart 28, control unit 32 drives electrode device 22 to selectively recruit nerve fibers beginning with smaller-diameter fibers and to progressively recruit larger-diameter fibers as the desired stimulation level increases. This smaller-to-larger diameter recruitment order mimics the body's natural order of recruitment.

Typically, in order to achieve this recruitment order, the control unit stimulates myelinated fibers essentially of all diameters using cathodic current from cathode 46, while simultaneously inhibiting fibers in a larger-to-smaller diameter order using efferent anodal current from efferent anode set 44. For example, FIG. 4 illustrates the recruitment of a single, smallest nerve fiber 56, without the recruitment of any larger fibers 50, 52 and 54. The depolarizations generated by cathode 46 stimulate all of the nerve fibers shown, producing action potentials in both directions along all the nerve fibers. Efferent anode set 44 generates a hyperpolarization effect sufficiently strong to block only the three largest nerve fibers 50, 52 and 54, but not fiber 56. This blocking order of larger-to-smaller diameter fibers is achieved because larger nerve fibers are inhibited by weaker anodal currents than are smaller nerve fibers. Stronger anodal currents inhibit progressively smaller nerve fibers. When the action potentials induced by cathode 46 in larger fibers 50, 52 and 54 reach the hyperpolarized region in the larger fibers adjacent to efferent anode set 44, these action potentials are blocked. On the other hand, the action potentials induced by cathode 46 in smallest fiber 56 are not blocked, and continue traveling unimpeded toward heart 28. Anode pole 44a is shown generating less current than anode pole 44b in order to minimize the virtual cathode effect in the direction of the heart, as described above.

When desired, in order to increase the parasympathetic stimulation delivered to the heart, the number of fibers not blocked is progressively increased by decreasing the amplitude of the current applied by efferent anode set 44. The action potentials induced by cathode 46 in the fibers now not blocked travel unimpeded towards the heart. As a result, the parasympathetic stimulation delivered to the heart is progressively increased in a smaller-to-larger diameter fiber order, mimicking the body's natural method of increasing stimulation. Alternatively or additionally, in order to increase the number of fibers stimulated, while simultaneously decreasing the average diameter of fibers stimulated, the amplitudes of the currents applied by cathode 46 and efferent anode set 44 are both increased (thereby increasing both the number of fibers stimulated and number of fibers blocked). In addition, for any given number of fibers stimulated (and not blocked), the amount of stimulation delivered to the heart can be increased by increasing the PPT, frequency, and/or pulse width of the current applied to vagus nerve 24.

In order to suppress artificially-induced afferent action potentials from traveling towards the brain in response to the cathodic stimulation, control unit 32 typically drives electrode device 22 to inhibit fibers 50, 52, 54 and 56 using afferent anodal current from afferent anode set 45. When the afferent-directed action potentials induced by cathode 46 in all of the fibers reach the hyperpolarized region in all of the fibers adjacent to afferent anode set 45, the action potentials are blocked. Blocking these afferent action potentials generally minimizes any unintended side effects, such as undesired or counterproductive feedback to the brain, that might be caused by these action potentials. Anode 45b is shown generating less current than anode 45a in order to minimize the virtual cathode effect in the direction of the brain, as described above.

In an embodiment of the present invention, the amplitude of the cathodic current applied in the vicinity of the vagus nerve is between about 2 mA and about 10 mA. Such a current is typically used in embodiments that employ techniques for achieving generally uniform stimulation of the vagus nerve, i.e., stimulation in which the stimulation applied to fibers on or near the surface of the vagus nerve is generally no more than about 400% greater than stimulation applied to fibers situated more deeply in the nerve. This corresponds to stimulation in which the value of the activation function at fibers on or near the surface of the vagus nerve is generally no more than about four times greater than the value of the activation function at fibers situated more deeply in the nerve. For example, as described hereinabove with reference to FIG. 2A, the electrodes may be recessed so as not to come in direct contact with vagus nerve 24, in order to achieve generally uniform values of the activation function. Typically, but not necessarily, embodiments using approximately 5 mA of cathodic current have the various electrodes disposed approximately 0.5 to 2.5 mm from the axis of the vagus nerve. Alternatively, larger cathodic currents (e.g., 10-30 mA) are used in combination with electrode distances from the axis of the vagus nerve of greater than 2.5 mm (e.g., 2.5-4.0 mm), so as to achieve an even greater level of uniformity of stimulation of fibers in the vagus nerve.

In an embodiment of the present invention, the cathodic current is applied by cathode 46 with an amplitude sufficient to induce action potentials in large- and medium-diameter fibers 50, 52, and 54 (e.g., A- and B-fibers), but insufficient to induce action potentials in small-diameter fibers 56 (e.g., C-fibers). Simultaneously, an anodal current is applied by anode 44b in order to inhibit action potentials induced by the cathodic current in the large-diameter fibers (e.g., A-fibers). This combination of cathodic and anodal current generally results in the stimulation of medium-diameter fibers (e.g., B-fibers) only. At the same time, a portion of the afferent action potentials induced by the cathodic current are blocked by anode 45a, as described above. Alternatively, the afferent anodal current is configured to not fully block afferent action potentials, or is simply not applied. In these cases, artificial afferent action potentials are nevertheless generally not generated in C-fibers, because the applied cathodic current is not strong enough to generate action potentials in these fibers.

These techniques for efferent stimulation of only B-fibers are typically used in combination with techniques described hereinabove for achieving generally uniform stimulation of the vagus nerve. Such generally uniform stimulation enables the use of a cathodic current sufficiently weak to avoid stimulation of C-fibers near the surface of the nerve, while still sufficiently strong to stimulate B-fibers, including B-fibers situated more deeply in the nerve, i.e., near the center of the nerve. For some applications, when employing such techniques for achieving generally uniform stimulation of the vagus nerve, the amplitude of the cathodic current applied by cathode 46 may be between about 3 and about 10 mA, and the amplitude of the anodal current applied by anode 44b may be between about 1 and about 7 mA. (Current applied at a different site and/or a different time is used to achieve a net current injection of zero.)

For some applications, control unit 32 is adapted to receive feedback from one or more of the electrodes in electrode device 22, and to regulate the signals applied to the electrode device responsive thereto. For example, control unit 32 may analyze amplitudes of various peaks in a compound action potential (CAP) signal recorded by the electrodes, in order to determine a relative proportion of stimulated larger fibers (having faster conduction velocities) to smaller fibers (having slower conduction velocities). Alternatively or additionally, control unit 32 analyzes an area of the CAP, in order to determine an overall effect of the stimulation. In an embodiment, the feedback is received by electrodes other than those used to apply signals to the nerve.

In an embodiment of the present invention, stimulation of the vagus nerve is applied responsive to one or more sensed parameters. Control unit 32 is typically configured to commence or halt stimulation, or to vary the amount and/or timing of stimulation in order to achieve a desired target heart rate, typically based on configuration values and on parameters including one or more of the following:
Heart rate—the control unit can be configured to drive electrode device 22 to stimulate the vagus nerve only when the heart rate exceeds a certain value.
ECG readings—the control unit can be configured to drive electrode device 22 to stimulate the vagus nerve based on certain ECG readings, such as readings indicative of designated forms of arrhythmia. Additionally, ECG readings are typically used for achieving a desire heart rate, as described below with reference to FIG. 5.
Blood pressure—the control unit can be configured to regulate the current applied by electrode device 22 to the vagus nerve when blood pressure exceeds a certain threshold or falls below a certain threshold.
Indicators of decreased cardiac contractility—these indicators include left ventricular pressure (LVP). When LVP and/or d(LVP)/dt exceeds a certain threshold or falls below a certain threshold, control unit 32 can drive electrode device 22 to regulate the current applied by electrode device 22 to the vagus nerve.
Motion of the subject—the control unit can be configured to interpret motion of the subject as an indicator of increased exertion by the subject, and appropriately reduce parasympathetic stimulation of the heart in order to allow the heart to naturally increase its rate.
Heart rate variability—the control unit can be configured to drive electrode device 22 to stimulate the vagus nerve based on heart rate variability, which is typically calculated based on certain ECG readings.
Norepinephrine concentration—the control unit can be configured to drive electrode device 22 to stimulate the vagus nerve based on norepinephrine concentration.
Cardiac output—the control unit can be configured to drive electrode device 22 to stimulate the vagus nerve based on cardiac output, which is typically determined using impedance cardiography.
Baroreflex sensitivity—the control unit can be configured to drive electrode device 22 to stimulate the vagus nerve based on baroreflex sensitivity.
LVEDP—the control unit can be configured to drive electrode device 22 to stimulate the vagus nerve based on LVEDP, which is typically determined using a pressure gauge, as described hereinabove with reference to FIG. 1.

The parameters and behaviors included in this list are for illustrative purposes only, and other possible parameters and/or behaviors will readily present themselves to those skilled in the art, having read the disclosure of the present patent application.

In an embodiment of the present invention, control unit 32 is configured to drive electrode device 22 to stimulate the vagus nerve so as to reduce the heart rate of the subject towards a target heart rate. The target heart rate is typically (a) programmable or configurable, (b) determined responsive to one or more sensed physiological values, such as those described hereinabove (e.g., motion, blood pressure, etc.), and/or (c) determined responsive to a time of day or circadian cycle of the subject. Parameters of stimulation are varied in real time in order to vary the heart-rate-lowering effects of the stimulation.

For example, such parameters may include the amplitude of the applied current. Alternatively or additionally, in an embodiment of the present invention, the stimulation is applied in bursts (i.e., series of pulses), which are synchronized or are not synchronized with the cardiac cycle of the subject, such as described hereinbelow with reference to FIG. 5. Parameters of such bursts typically include, but are not limited to:
Timing of the stimulation within the cardiac cycle. Delivery of each of the bursts typically begins after a fixed or variable delay following an ECG feature, such as each R- or P-wave. For some applications, the delay is between about 20 ms and about 300 ms from the R-wave, or between about 100 and about 500 ms from the P-wave.
Pulse duration (width). Longer pulse durations typically result in a greater heart-rate-lowering effect. For some applications, the pulse duration is between about 0.2 and about 4 ms.
Pulse repetition interval within each burst. Maintaining a pulse repetition interval (the time from the initiation of a pulse to the initiation of the following pulse within the same burst) greater than about 3 ms generally results in maximal stimulation effectiveness for multiple pulses within a burst. For some applications, the pulse repetition interval is between about 3 and about 10 ms.
Pulses per trigger (PPT). A greater PPT (the number of pulses in each burst after a trigger such as an R-wave) typically results in a greater heart-rate-lowering effect. For some applications, PPT is between about 0 and about 8. For some applications, PPT is varied while pulse repetition interval is kept constant.
Amplitude. A greater amplitude of the signal applied typically results in a greater heart-rate-lowering effect. The amplitude is typically less than about 10 milliamps, e.g., between about 2 and about 10 milliamps. For some applications, the amplitude is between about 2 and about 6 milliamps.
Duty cycle (number of bursts per heart beat). Application of stimulation every heartbeat (i.e., with a duty cycle of 1) typically results in a greater heart-rate-lowering effect. For less heart rate reduction, stimulation is applied less frequently than every heartbeat (e.g., duty cycle=60%-90%), or only once every several heartbeats (e.g., duty cycle=5%-40%).
Choice of vagus nerve. Stimulation of the right vagus nerve typically results in greater heart rate reduction than stimulation of the left vagus nerve.
“On”/“off” ratio and timing. For some applications, the device operates intermittently, alternating between “on” and “off” states, the length of each state typically being between 0 and about 1 day, such as between 0 and about 300 seconds (with a O-length “off” state equivalent to always “on”). No stimulation is applied during the “off” state. Greater heart rate reduction is typically achieved if the device is “on” a greater portion of the time.

For some applications, values of one or more of the parameters are determined in real time using feedback (i.e., responsive to one or more inputs). The inputs typically include sensed physiological values, such as:
a temperature of the subject;
a blood glucose level of the subject;
a blood lipid level of the subject;
a blood lactic acid level of the subject;
a blood CO₂ or O₂ level of the subject; and/or
a blood urea, creatinine, or ammonia level of the subject.

For some applications, values of one or more of the parameters are set responsively to one or more inputs. The inputs may include, for example, a signal generated by the subject, such as by applying a magnet, or sending a wireless command to change a parameter value. For some applications, the patient sends such a signal to signify:
a convenient or inconvenient time for stimulation;
that the patient is taking a drug;
that the patient is undergoing dialysis;
that the patient is performing exercise;
that the patient is going to sleep or awakening; and/or
that the patient is experiencing a subjective feeling of a habitual need.

For some applications, an intermittency (“on”/“off”) parameter is determined in real time using such feedback. The inputs used for such feedback typically include one or more of the following: (a) motion or activity of the subject (e.g., detected using an accelerometer), (b) the average heart rate of the subject, (c) the average heart rate of the subject when the device is in “off” mode, (d) the average heart rate of the subject when the device is in “on” mode, and/or (e) the time of day. The average heart rate is typically calculated over a period of at least about 10 seconds. For some applications, the average heart rate during an “on” or “off” period is calculated over the entire “on” or “off” period. For example, the device may operate in continuous “on” mode when the subject is exercising and therefore has a high heart rate, and the device may alternate between “on” and “off” when the subject is at rest. As a result, the heart-rate-lowering effect is concentrated during periods of high heart rate, and the nerve is allowed to rest when the heart rate is generally naturally lower. For some applications, the device determines the ratio of “on” to “off” durations, the duration of the “on” periods, and/or the durations of the “off” periods using feedback. Optionally, the device determines the “on”/“off” parameter in real time using the integral feedback techniques described hereinbelow, and/or other feedback techniques described hereinbelow, mutatis mutandis.

For some applications, heart rate regulation is achieved by setting two or more parameters in combination. For example, if it is desired to apply 5.2 pulses of stimulation, the control unit may apply 5 pulses of 1 ms duration each, followed by a single pulse of 0.2 ms duration. For other applications, the control unit switches between two values of PPT, so that the desired PPT is achieved by averaging the applied PPTs. For example, a sequence of PPTs may be 5, 5, 5, 5, 6, 5, 5, 5, 5, 6, . . . , in order to achieve an effective PPT of 5.2.

In an embodiment of the present invention, the heart rate regulation algorithm is implemented using only integer arithmetic. For example, division is implemented as integer division by a power of two, and multiplication is always of two 8-bit numbers. For some applications, time is measured in units of 1/128 of a second.

In an embodiment of the present invention, control unit 32 implements an integral feedback controller, which can most generally be described by:

K=K_{I}*∫edt

in which K represents the strength of the feedback, K_{I} is a coefficient, and ∫e dt represents the cumulative error. It is to be understood that such an integral feedback controller can be implemented in hardware, or in software running in control unit 32.

In an embodiment of such an integral controller, heart rate is typically expressed as an R-R interval (the inverse of heart rate). Parameters of the integral controller typically include TargetRR (the target R-R interval) and TimeCoeff (which determines the overall feedback reaction time).

Typically, following the detection of each R-wave, the previous R-R interval is calculated and assigned to a variable (LastRR). e (i.e., the difference between the target R-R interval and the last measured R-R interval) is then calculated as:

e=TargetRR−LastRR

e is typically limited by control unit 32 to a certain range, such as between −0.25 and +0.25 seconds, by reducing values outside the range to the endpoint values of the range. Similarly, LastRR is typically limited, such as to 255/128 seconds. The error is then calculated by multiplying LastRR by e:

Error=e*LastRR

A cumulative error (representing the integral in the above generalized equation) is then calculated by dividing the error by TimeCoeff and adding the result to the cumulative error, as follows:

Integral=Integral+Error/2^{TimeCoeff}

The integral is limited to positive values less than, e.g., 36,863. The number of pulses applied in the next series of pulses (pulses per trigger, or PPT) is equal to the integral/4096.

The following table illustrates example calculations using a heart rate regulation algorithm that implements an integral controller, in accordance with an embodiment of the present invention. In this example, the parameter TargetRR (the target heart rate) is set to 1 second (128/128 seconds), and the parameter TimeCoeff is set to 0. The initial value of Integral is 0. As can be seen in the table, the number of pulses per trigger (PPT) increases from 0 during the first heart beat, to 2 during the fourth heart beat of the example.

Heart Beat Number

| | 1 | 2 | 3 | 4 |
|---|---|---|---|---|
| Heart rate (BPM) | 100 | 98 | 96 | 102 |
| R-R interval (ms) | 600 | 610 | 620 | 590 |
| R-R ( 1/128 sec) | 76 | 78 | 79 | 75 |
| e ( 1/128 sec) | 52 | 50 | 49 | 53 |
| Limited e | 32 | 32 | 32 | 32 |
| Error | 2432 | 2496 | 2528 | 2400 |
| Integral | 2432 | 4928 | 7456 | 9856 |
| PPT | 0 | 1 | 1 | 2 |

In an embodiment of the present invention, the heart rate regulation algorithm corrects for missed heart beats (either of physiological origin or because of a failure to detect a beat). Typically, to perform this correction, any R-R interval which is about twice as long as the immediately preceding R-R interval is interpreted as two R-R intervals, each having a length equal to half the measured interval. For example, the R-R interval sequence (measured in seconds) 1, 1, 1, 2.2 is interpreted by the algorithm as the sequence 1, 1, 1, 1.1, 1.1. Alternatively or additionally, the algorithm corrects for premature beats, typically by adjusting the timing of beats that do not occur approximately halfway between the preceding and following beats. For example, the R-R interval sequence (measured in seconds) 1, 1, 0.5, 1.5 is interpreted as 1, 1, 1, 1, using the assumption that the third beat was premature.

In an embodiment of the present invention, control unit 32 is configured to operate in one of the following modes:
vagal stimulation is not applied when the heart rate of the subject is lower than the low end of the normal range of a heart rate of the subject and/or of a typical human subject;
vagal stimulation is not applied when the heart rate of the subject is lower than a threshold value equal to the current low end of the range of the heart rate of the subject, i.e., the threshold value is variable over time as the low end generally decreases as a result of chronic vagal stimulation treatment;
vagal stimulation is applied only when the heart rate of the subject is within the normal of range of a heart rate of the subject and/or of a typical human subjects;
vagal stimulation is applied only when the heart rate of the subject is greater than a programmable threshold value, such as a rate higher than a normal rate of the subject and/or a normal rate of a typical human subject. This mode generally removes peaks in heart rate; or
vagal stimulation is applied using fixed programmable parameters, i.e., not in response to any feedback, target heart rate, or target heart rate range. These parameters may be externally updated from time to time, for example by a physician.

In an embodiment of the present invention, the amplitude of the applied stimulation current is calibrated by fixing a number of pulses in the series of pulses (per cardiac cycle), and then increasing the applied current until a desired pre-determined heart rate reduction is achieved. Alternatively, the current is calibrated by fixing the number of pulses per series of pulses, and then increasing the current to achieve a substantial reduction in heart rate, e.g., 40%.

In embodiments of the present invention in which apparatus 20 comprises an implanted device for monitoring and correcting the heart rate, control unit 32 typically uses measured parameters received from the device as additional inputs for determining the level and/or type of stimulation to apply. Control unit 32 typically coordinates its behavior with the behavior of the device. Control unit 32 and the device typically share sensors 40 in order to avoid redundancy in the combined system.

Optionally, apparatus 20 comprises a patient override, such as a switch that can be activated by the subject using an external magnet. The override typically can be used by the subject to activate vagal stimulation, for example in the event of arrhythmia apparently undetected by the system, or to deactivate vagal stimulation, for example in the event of apparently undetected physical exertion.

FIG. 5 is a simplified illustration of an ECG recording 70 and example timelines 72 and 76 showing the timing of the application of a burst of stimulation pulses 74, in accordance with an embodiment of the present invention. The application of the burst of pulses in each cardiac cycle typically commences after a variable delay after a detected R-wave, P-wave, or other feature of an ECG. For some applications, other parameters of the applied burst of pulses are also varied in real time. Such other parameters include amplitude, pulses per trigger (PPT), pulse duration, and PRI. For some applications, the delay and/or one or more of the other parameters are calculated in real time using a function, the inputs of which include one or more pre-programmed but updateable constants and one or more sensed parameters, such as the R-R interval between cardiac cycles and/or the P-R interval.

The variable delay before applying pulse burst 74 in each cardiac cycle can be measured from a number of sensed physiological parameters (“initiation physiological parameters”), including sensed points in the cardiac cycle, including P-, Q-, R-, S- and T-waves. Typically the delay is measured from the P-wave, which indicates atrial contraction. Alternatively, the delay is measured from the R-wave, particularly when the P-wave is not easily detected. Timeline A 72 and Timeline B 76 show the delays, dt_{R} and dtp measured from R and P, respectively.

In an embodiment, a lookup table of parameters, such as delays (e.g., dt) and/or other parameters, is used to determine in real time the appropriate parameters for each application of pulses, based on the one or more sensed parameters, and/or based on a predetermined sequence stored in the lookup table. For example, in embodiments of the present invention in which the control unit configures signals applied to the vagus nerve so as to induce cardioversion, such a predetermined sequence may include delays of alternating longer and shorter durations.

Optionally, the stimulation applied by stimulation apparatus 20 is applied in conjunction with or separately from stimulation of sympathetic nerves innervating the heart. For example, inhibition described herein and/or periods of non-stimulation described herein may be replaced or supplemented by excitation of sympathetic nerves. Such sympathetic stimulation can be applied using techniques of smaller-to-larger diameter fiber recruitment, as described herein, or other nerve stimulation techniques known in the art. For some applications, vagal or other parasympathetic stimulation is applied in conjunction with stimulation of sympathetic nerves in order to increase vagal tone while minimizing the heart-rate-lowering effect of the parasympathetic stimulation.

For some applications, stimulation is applied to vagus nerve 24 in a closed-loop system in order to achieve and maintain the desired target heart rate, determined as described above. Precise graded slowing of the heart beat is typically achieved by varying the number of nerve fibers stimulated, in a smaller-to-larger diameter order, and/or the intensity of vagus nerve stimulation, such as by changing the stimulation amplitude, pulse width, PPT, and/or delay. Stimulation with blocking, as described herein, is typically applied during each cardiac cycle in burst of pulses 74, typically containing between about 1 and about 20 pulses, each of about 1-3 milliseconds duration, over a period of about 1-200 milliseconds. Advantageously, such short pulse durations generally do not substantially block or interfere with the natural efferent or afferent action potentials traveling along the vagus nerve. Additionally, the number of pulses and/or their duration is sometimes varied in order to facilitate achievement of precise graded slowing of the heart beat.

Alternatively or additionally, the techniques of smaller-to-larger diameter fiber recruitment are applied in conjunction with methods and apparatus described in one or more of the patents, patent applications, articles and books cited herein.

Reference is made to FIG. 6, which is a schematic illustration of a series of bursts 60, in accordance with an embodiment of the present invention. Control unit 32 is configured to drive electrode device 22 to apply stimulation, such as for reducing the risk of AF, as described herein, in the series of bursts 60, at least one of which bursts includes a plurality of pulses 62, such as at least three pulses 62. Control unit 32 configures:
(a) a pulse repetition interval (PRI) within each of multi-pulse bursts 60 (i.e., the time from the initiation of a pulse to the initiation of the following pulse within the same burst) to be on average at least 20 ms, such as at least 30 ms, e.g., at least 50 ms or at least 75 ms, and
(b) an interburst interval (II) (i.e., the time from the initiation of a burst to the initiation of the following burst) to be at least a multiple M times the burst duration D. Multiple M is typically at least 1.5 times the burst duration D, such as at least 2 times the burst duration, e.g., at least 3 or 4 times the burst duration. (Burst duration D is the time from the initiation of the first pulse within a burst to the conclusion of the last pulse within the burst.)

In other words, burst duration D is less than a percentage P of interburst interval II, such as less than 75%, e.g., less than 67%, 50%, or 33% of the interval. For some applications, the PRI varies within a given burst, in which case the control unit sets the PRI to be on average at least 20 ms, such as at least 30 ms, e.g., at least 50 ms or at least 75 ms. For other applications, the PRI does not vary within a given burst (it being understood that for these applications, the “average PRI” and the PRI “on average,” including as used in the claims, is equivalent to the PRI; in other words, the terms “average PRI” and the PRI “on average” include within their scope both (a) embodiments with a constant PRI within a given burst, and (b) embodiments with a PRI that varies within a given burst).

Typically, each burst 60 includes between two and 14 pulses 62, e.g., between two and six pulses, and the pulse duration (or average pulse duration) is between about 0.1 and about 4 ms, such as between about 100 microseconds and about 2.5 ms, e.g., about 1 ms. Typically, control unit 32 sets the interburst interval II to be less than 10 seconds. For some applications, control unit 32 is configured to set the interburst interval II to be between 400 ms and 1500 ms, such as between 750 ms and 1500 ms. Typically, control unit 32 sets an interburst gap G between a conclusion of each burst 60 and an initiation of the following burst 60 to have a duration greater than the PRI. For some applications, the duration of the interburst gap G is at least 1.5 times the PRI, such as at least 2 times the PRI, at least 3 times the PRI, or at least 4 times the PRI.

Although the control unit typically withholds applying current during the periods between bursts and between pulses, it is to be understood that the scope of the present invention includes applying a low level of current during such periods, such as less than 50% of the current applied during the “on” periods, e.g., less than 20% or less than 5%. Such a low level of current is hypothesized to have a different, significantly lower, or a minimal physiological effect on the subject. For some applications, control unit 32 is configured to apply an interburst current during at least a portion of interburst gap G, and to set the interburst current on average to be less than 50% (e.g., less than 20%) of the current applied on average during the burst immediately preceding the gap. For some applications, control unit 32 is configured to apply an interpulse current to the site during at least a portion of the time that the pulses of bursts 60 are not being applied, and to set the interpulse current on average to be less than 50% (e.g., less than 20%) of the current applied on average during bursts 60.

For some applications, the control unit is configured to synchronize the bursts with a feature of the cardiac cycle of the subject. For example, each of the bursts may commence after a delay after a detected R-wave, P-wave, or other feature of an ECG. For these applications, one burst is typically applied per heart beat, so that the interburst interval II equals the R-R interval, or a sum of one or more sequential R-R intervals of the subject. Alternatively, for some applications, the control unit is configured to synchronize the bursts with other physiological activity of the subject, such as respiration, muscle contractions, or spontaneous nerve activity.

In an embodiment of the present invention, the control unit sets the PRI to at least 75% of a maximum possible PRI for a given interburst interval II (such as the R-R interval of the subject), desired percentage P, and desired PPT. For some applications, the following equation is used to determine the maximum possible PRI:

PRI=II*P/(PPT−1) (Equation 1)

For example, if the II is 900 ms, percentage P is 33.3%, and the desired PPT is 4 pulses, the maximum possible PRI would be 900 ms*33.3%/(4−1)=100 ms, and the control unit would set the actual PRI to be at least 75 ms. For some applications, control unit 32 uses this equation to determine the PRI, such as in real time or periodically, while for other applications this equation is used to produce a look-up table which is stored in the control unit. For still other applications, this equation is used to configure the control unit. For some applications, multiple M is a constant, which is stored in control unit 32, while for other applications, control unit 32 adjusts M during operation, such as responsively to one or more sensed physiological values, or based on the time of day, for example. It is noted that Equation 1 assumes that the pulse width of the pulses does not contribute meaningfully to burst duration D. Modifications to Equation 1 to accommodate longer pulse widths will be evident to those skilled in the art.

For some applications, when using Equation 1, a maximum value is set for the PRI, such as between 175 and 225, e.g., about 200, and the PRI is not allowed to exceed this maximum value regardless of the result of Equation 1.

Reference is made to FIG. 7, which is a schematic illustration of a stimulation regimen, in accordance with an embodiment of the present invention. Control unit 32 is configured to apply the stimulation, such as for reducing the risk of AF, as described herein, during “on” periods 100 alternating with “off” periods 102, during which no stimulation is applied (each set of a single “on” period followed by a single “off” period is referred to hereinbelow as a “cycle” 104). Typically, each of “on” periods 100 has an “on” duration equal to at least 1 second (e.g., between 1 and 10 seconds), and each of “off” periods 102 has an “off” duration equal to at least 50% of the “on” duration, e.g., at least 100% or 200% of the “on” duration. Control unit 32 is further configured to apply such intermittent stimulation during stimulation periods 110 alternating with rest periods 112, during which no stimulation is applied. Each of rest periods 102 typically has a duration equal to at least the duration of one cycle 104, e.g., between one and 50 cycles, such as between two and four cycles, and each of stimulation periods 110 typically has a duration equal to at least 5 times the duration of one of rest periods 112, such as at least 10 times, e.g., at least 15 times. For example, each of stimulation periods 110 may have a duration of at least 30 cycles, e.g., at least 60 cycles or at least 120 cycles, and no greater than 2400 cycles, e.g., no greater than 1200 cycles. Alternatively, the duration of the stimulation and rest periods are expressed in units of time, and each of the rest periods has a duration of at least 30 seconds, e.g., such as at least one minute, at least two minutes, at least five minutes, or at least 25 minutes, and each of the stimulation periods has a duration of at least 10 minutes, e.g., at least 30 minutes, such as at least one hour, and less than 12 hours, e.g., less than six hours, such as less than two hours.

For some applications, low stimulation periods are used in place of “off” periods 102. During these low stimulation periods, the control unit sets the average current applied to be less than 50% of the average current applied during the “on” periods, such as less than 20% or less than 5%. Similarly, for some applications, the control unit is configured to apply a low level of current during the rest periods, rather than no current. For example, the control unit may set the average current applied during the rest periods to be less than 50% of the average current applied during the “on” periods, such as less than 20% or less than 5%. As used in the present application, including in the claims, the “average current” or “current applied on average” during a given period means the total charge applied during the period (which equals the integral of the current over the period, and may be measured, for example, in coulombs) divided by the duration of the period, such that the average current may be expressed in mA, for example.

For some applications, these rest period stimulation techniques are combined with the extended PRI techniques described hereinabove with reference to FIG. 6.

Reference is made to FIG. 8, which is a schematic illustration of a stimulation regimen, in accordance with an embodiment of the present invention. In this embodiment, control unit 32 is configured to apply stimulation, such as for reducing the risk of AF, as described herein, in a series of bursts 200, each of which includes one or more pulses 202 (pulses per trigger, or PPT). The control unit is configured to apply the stimulation intermittently during “on” periods 204 alternating with “off” periods 206, during which no stimulation is applied. Each “on” period 204 includes at least 3 bursts 200, such as at least 10 bursts 200, and typically has a duration of between 3 and 20 seconds. At the commencement of each “on” period 204, control unit 32 ramps up the PPT of successive bursts 200, and at the conclusion of each “on” period 204, the control unit ramps down the PPT of successive bursts 200. For example, the first four bursts of an “on” period 204 may have respective PPTs of 1, 2, 3, and 3, or 1, 2, 3, and 4, and the last four bursts of an “on” period 204 may have respective PPTs of 3, 3, 2, and 1, or 4, 3, 2, and 1.

Alternatively, rather than increase or decrease the PPT by 1 in successive bursts, control unit 32 increases or decreases the PPT more gradually, such as by 1 in less than every successive burst, e.g., the first bursts of an “on” period may have respective PPTs of 1, 1, 2, 2, 3, 3, and 4, and the last bursts of an “on” period may have respective PPTs of 4, 3, 3, 2, 2, 1, and 1. For some applications, to increase or decrease the PPT by less than 1 in successive bursts, the control unit increases or decreases the PPT by non-integer values, and achieves the non-integer portion of the increase or decrease by setting a parameter of one or more pulses other than PPT, such as pulse duration or amplitude. For example, the first bursts of an “on” period may have respective PPTs of 0.5, 1, 1.5, 2, 2.5, and 3, and the last bursts of an “on” period may have respective PPTs of 3, 2.5, 2, 1.5, 1, and 0.5. To achieve the decimal portion of these PPTs, the control unit may apply a pulse having a pulse duration equal to the decimal portion of these PPTs times the pulse duration of a full pulse. For example, if the pulse duration of a full pulse is 1 ms, a commencement ramp of 0.5, 1, and 1.5 PPT may be achieved by applying a first burst consisting of a single 0.5 ms pulse, a second burst consisting of a single 1 ms pulse, and a third burst consisting of a 1 ms pulse followed by a 0.5 ms pulse. Alternatively, to achieve the decimal portion of these PPTs, the control unit may apply a pulse having a full pulse duration but an amplitude equal to the decimal portion of these PPTs times the amplitude of a full pulse. For example, if the pulse duration and amplitude of a full pulse if 1 ms and 3 mA, respectively, a commencement ramp of 0.5, 1, and 1.5 PPT may be achieved by apply a first burst consisting of a single 1 ms pulse having an amplitude of 1.5 mA, a second burst consisting of a single 1 ms, 3 mA pulse, and a third burst consisting of a 1 ms, 3 mA followed by a 1 ms pulse having an amplitude of 1.5 mA.

For some applications, control unit 32 is configured to synchronize the bursts with a feature of the cardiac cycle of the subject. For example, each of the bursts may commence after a delay after a detected R-wave, P-wave, or other feature of an ECG. Alternatively, for some applications, the control unit is configured to synchronize the bursts with other physiological activity of the subject, such as respiration, muscle contractions, or spontaneous nerve activity. For some applications, such ramping is applied only at the commencement of each “on” period 204, or only at the conclusion of each “on” period 204, rather than during both transitional periods.

For some applications, such ramping techniques are combined with the extended PRI techniques described hereinabove with reference to FIG. 6, and/or with the rest period techniques described hereinabove with reference to FIG. 7.

Reference is now made to FIG. 9, which is a graph showing in vivo experimental results measured in accordance with an embodiment of the present invention. A SABAR white rat, weighing 350 g, was anesthetized with Phenobarbital; no other medications were administered. Vagal stimulation was applied using a silver chloride hook electrode immersed in oil placed over the right vagus nerve.

The graph of FIG. 9 shows change in heart rate vs. baseline heart rate, as measured over a 300 second period. During the entire period of the experiment, vagal stimulation was applied in 500 microsecond pulses having an amplitude of 4 mA, at a frequency of 8 Hz. The stimulation was not synchronized with the cardiac cycle of the animal. Beginning at 0 seconds, and concluding at about 12 seconds, 0.8 mg per kg body weight of atropine was administered by intravenous injection to the tail vein.

During the approximately 12 seconds of atropine administration, prior to the atropine taking effect, vagal stimulation is seen demonstrating its expected heart-rate lowering effect, which is attributable to the parasympathetic effect of such stimulation. However, beginning at approximately 13 seconds, with the onset of the effectiveness of the atropine, the heart rate suddenly increased to a level that varied between about 0 and about 20 beats per minute greater than baseline heart rate. This increase is attributed to the fact that vagal stimulation generally has both a parasympathetic and adrenergic effect. Under normal circumstances, the parasympathetic effect dominates the adrenergic effect. However, when the parasympathetic effect is blocked, such as by atropine, the adrenergic effect is expressed, resulting in increased heart rate, among other effects. Beginning at about 180 seconds, as the atropine-induced parasympathetic blockade faded, the parasympathetic effect of stimulation again began to dominate, resulting in a reduced heart rate.

It is believed by the inventors that these experimental results at least in part explain the effectiveness of the minimal heart rate reduction stimulation described hereinabove. During stimulation with such parameters, the heart-rate-lowering effects of vagal stimulation are nearly offset by the adrenergic effects of the vagal stimulation. Nevertheless, the parasympathetic nervous system is still activated, resulting in the beneficial effects of such stimulation described hereinabove.

FIG. 10 is a graph showing in vivo experimental results measured in accordance with an embodiment of the present invention. A male dog, weighing 25 kg, was initially anesthetized with propafol; anesthesia was maintained with inhaled gas isoflurane. The dog was mechanically ventilated. The right vagus nerve was stimulated using a tripolar cuff electrode in an anode-cathode-anode configuration, with the anodes shorted to each other, similar to the shorted anode configuration described hereinabove with reference to FIG. 2A. The cuff electrode was immersed in normal saline solution.

The graph of FIG. 10 shows heart rate reduction vs. baseline (with reduction expressed by positive values) responsive to vagal stimulation applied after different delays from the R-wave. Baseline heart rate was calculated based on the average interval between beats prior to beginning stimulation. For each data point, the heart rate was calculated as the time interval between the second and third beat after application of the stimulation. The reduction in heart rate caused by the stimulation is shown on the y-axis. As is seen in the graph, longer delays from the R-wave generally resulted in less heart rate reduction. Delays of at least 200 milliseconds resulted in substantially no reduction in heart rate. It is believed by the inventors that these data support the timing parameters of the minimal heart rate reduction stimulation described hereinabove. It is hypothesized by the inventors that for each of the delays shown, total acetylcholine release is substantially the same. In support of this hypothesis, it is noted that acetylcholine is released in efferent fibers in response to the applied vagal stimulation, but is expected to be largely (or entirely) unaffected in these fibers by the precise timing of the cardiac cycle, because these fibers do not receive input from the heart. Because acetylcholine release is an indication of the level of parasympathetic stimulation, this hypothesis as well as the experimental results indicate that vagal stimulation, with delays chosen in accordance with this embodiment, has little or no effect on heart rate, while maintaining substantially the same effect on the parasympathetic nervous system.

In an embodiment of the present invention, a calibration period is provided to determine a delay for each patient that generally corresponds to, for example, the 200 ms delay shown in the figure, and this determined delay is applied to allow vagal stimulation with minimal heart rate reduction in the patient.

FIG. 11 is a chart showing in vivo experimental results in accordance with an embodiment of the present invention. A SABAR white rat, weighing 350 g, was anesthetized with Phenobarbital. Vagal stimulation was applied using a silver chloride hook electrode immersed in oil placed over the right vagus nerve. Vagal stimulation was applied with an amplitude of 1.5 milliamps. Medications, as described below, were administered intravenously through the tail vein.

The chart of FIG. 11 shows heart rate reductions vs. baseline heart rates (with the reductions expressed by positive values) responsive to vagal stimulation applied alone (bars 1), vagal stimulation applied after administration of 1 mg of the beta-blocker metoprolol (bars 2), and vagal stimulation applied after administration of 0.2 mg of adrenaline (bars 3). (For determining the metoprolol and adrenaline reductions, the respective baselines were measured after the medications had taken effect.) The left bar in each pair of bars shows results when vagal stimulation was synchronized with the cardiac cycle, and the right bar shows results with unsynchronized stimulation. As is seen, both the beta-blocker and adrenaline cause vagal stimulation to achieve a greater heart-rate-lowering effect at the same level of stimulation.

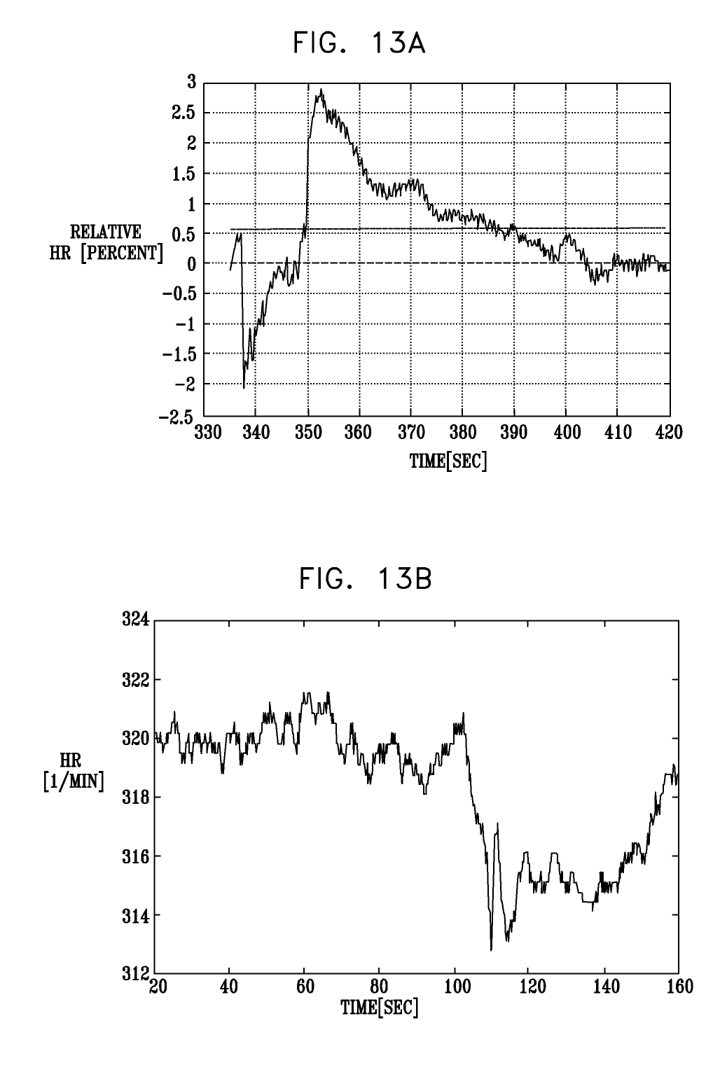

FIGS. 12A and 12B are graphs showing an analysis of the experimental results of the experiment described hereinabove with reference to FIG. 10, in accordance with an embodiment of the present invention. Both graphs show heart rate reduction vs. baseline (with reduction expressed by positive values). However, in FIG. 12A increased reduction was achieved by increasing the amplitude of the applied signal, while in FIG. 12B increased reduction was achieved by increasing the number of pulses per trigger (PPT), i.e., the number of pulses in a pulse train applied once per cardiac cycle. The pulses of the experiment shown in FIG. 12B were applied after a constant delay of 60 ms after each R-wave, synchronized with the cardiac cycle.

Although similar fine control of heart rate reduction was achieved using modulation of both parameters, the animal experienced severe side effects, including breathing difficulties (gasping, belching, hoarseness, and wheezing), when signal amplitude was modulated (FIG. 12A) and the heart rate reduction reached about 40 beats per minute. Substantially no side effects were observed when PPT was modulated. These data suggest that heart rate reduction can be achieved with fewer side effects by varying PPT rather than signal amplitude.

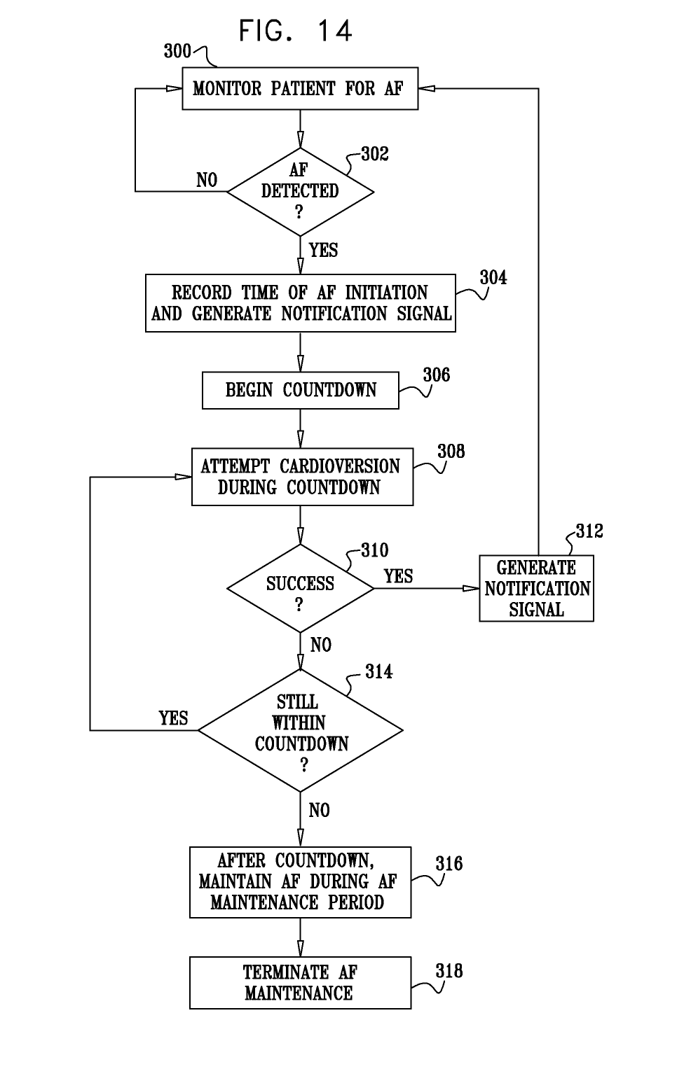

FIGS. 13A and 13B are graphs showing in vivo experimental results in accordance with an embodiment of the present invention. These graphs respectively reflect two different sets of parameters used to achieve vagal stimulation with minimal heart-rate-lowering effects. A SABAR white rat, weighing 350 g, was anesthetized with Phenobarbital; no other medications were administered. Vagal stimulation was applied using a silver chloride hook electrode immersed in oil placed over the right vagus nerve. The heart rate in FIG. 13A is expressed as a percent change from a baseline average heart rate.

The data shown in the graph of FIG. 13A were obtained using the following stimulation parameters: (a) an “on” time of 12.5 seconds (10 triggers), and an “off” time of 110 seconds, (b) 1 pulse per trigger, (c) a stimulation frequency of 0.8 Hz, (d) an amplitude of 2 milliamps, and (e) a pulse width of 500 microseconds. Stimulation was applied between about 336 and about 348.5 seconds. As seen in the graph, the stimulation initially reduced the heart rate (until about 350 seconds). However, upon cessation of stimulation at 348.5 seconds, heart rate increased with rebound strength for about 40 seconds (until about 390 seconds). As a result, the average heart rate caused by stimulation was not substantially different from the average heart rate without stimulation. This lack of substantial difference is illustrated by the two horizontal lines of the graph. The upper line represents the average heart rate during stimulation and the 40 seconds following stimulation (i.e., between 330 and 390 seconds), while the lower line represents the average heart rate excluding these periods (i.e., the average heart rate between 300 and 330 seconds, and between 390 and 420 seconds.

The data shown in the graph of FIG. 13B were obtained using the following stimulation parameters: (a) 1 pulse per trigger, (b) an amplitude of 0.1 mA, and (c) a pulse duration of 500 microseconds. Stimulation was applied at two stimulation time points, the first at 35 seconds and the second at 100 seconds. The stimulation applied at the first point consisted of 4 triggers (i.e., cardiac cycles), while the stimulation applied at the second point consisted of 12 triggers. As is shown on the graph, the stimulation applied at the first point had essentially no heart-rate-lowering effect, while the stimulation applied at the second point substantially lowered the heart rate. These results demonstrate that, mutatis mutandis, the heart-rate-lowering effect of vagal stimulation depends in part upon the length (i.e., number of triggers) of the stimulation. By using a brief stimulation period, vagal stimulation can be achieved while having a minimal or no heart-rate-lowering effect.

In an embodiment of the present invention, control unit 32 drives electrode device 22 to apply signals to vagus nerve 26, and configures the signals to maintain pre-existing AF, i.e., to prevent the return to normal sinus rhythm (NSR). Typically, stimulation is applied in bursts (i.e., a series of pulses), and typical signal parameters include a pulse amplitude of between about 2 and about 5 milliamps, such as about 3 milliamps, a pulse duration of between about 1 and about 3 milliseconds, such as about 2 milliseconds, a PPT of between about 1 and about 8 pulses per trigger, such as about 6 pulses per trigger, and a pulse repetition interval of between about 5 and about 90 milliseconds, such as about 70 milliseconds. Alternatively, the pulse duration is between about 0.5 and about 3 milliseconds, and/or the PPT is between about 1 and about 100 pulses per trigger. For some applications, a constant ventricular response is maintained, such as by using techniques described in the above-cited U.S. patent application Ser. No. 10/205,475, or by using other techniques known in the art. For some applications, if NSR returns despite vagal stimulation, the intensity of vagal stimulation is increased for a short period, in order to induce a return to AF. For example, the period may have a duration of about one minute, and the more intense stimulation may have an amplitude of 6 milliamps and a PPT of 6 pulses per trigger. Alternatively or additionally, vagal stimulation is applied, and/or the intensity of vagal stimulation is increased, upon detection of a complex in the subject's cardiac rhythm other than NSR. Further alternatively or additionally, stimulation is not synchronized with features of the cardiac cycle. In this case, example signal parameter include an amplitude of about 3 milliamps, a pulse width of about 1 millisecond, and a frequency of about 5 Hz.

Alternatively or additionally, in order to achieve AF maintenance, control unit 32 drives stimulator 34 to electrically stimulate cardiac tissue of patient 30, such as the fat pads or atrial tissue 37. Typically, the atria are rapidly electrically paced during such stimulation. Typically, the stimulation is applied at a frequency of at least about 3 Hz with an amplitude greater than the diastolic threshold.

Electrical techniques for initiating and maintaining AF in animals for experimental purposes are known in the art (see, for example, the following articles, all of which are incorporated herein by reference: (a) Friedrichs GS, “Experimental models of atrial fibrillation/flutter,” J Pharmacological and Toxicological Methods 43:117-123 (2000); (b) Morillo C A et al., “Chronic rapid atrial pacing. Structural, functional, and electrophysiological characteristics of a new model of sustained atrial fibrillation,” Circulation 91:1588-1595 (1995), and the above-cited article by Wijffels et al., entitled, “Atrial fibrillation begets atrial fibrillation”). For example, in the article entitled, “Atrial fibrillation begets atrial fibrillation,” Wijffels et al. describe a technique for initiating and maintaining AF in goats. In this technique, a set of recording electrodes and a set of stimulating electrodes are applied to both atria. An atrial cardiogram is continuously analyzed in order to distinguish between sinus rhythm and AF. When sinus rhythm is detected, a one-second burst of biphasic stimuli (having an interval of 20 ms, i.e., a frequency of 50 Hz, and four times diastolic threshold) is delivered using one or more of the stimulating electrodes. In an embodiment, this technique, with appropriate modifications for therapeutic application to human patients, is used to maintain AF in human patients suffering from AF for therapeutic purposes, as described herein. Other optional modifications of this technique include, but are not limited to:
using other techniques for detection of AF and NSR, such as those described hereinabove;
using other parameters for the applied stimuli. Typically, the stimulation is applied at a frequency of at least about 3 Hz; and/or
applying all or a portion of the stimulating electrodes to other cardiac tissue, such as the fat pads.

Other AF initiation/maintenance techniques known in the art (including those described in the above-mentioned Friedrichs GS and Morillo et al.), optionally with the modifications described immediately hereinabove, may also be used to maintain AF to treat AF in human patients.

In an embodiment of the present invention, AF maintenance is achieved by performing vagal stimulation in conjunction with cardiac tissue stimulation. Techniques for such dual stimulation may be used that are described in the above-cited articles by Friedrichs GS and Hayashi H et al., “Different effects of class Ic and III antiarrhythmic drugs on vagotonic atrial fibrillation in the canine heart,” Journal of Cardiovascular Pharmacology 31:101-107 (1998), which is incorporated herein by reference.

In another embodiment of the present invention, AF is maintained using surgical techniques, such as creating an electrical blockade in the atrium. Examples of such surgical techniques used in animal models are described in the above-referenced article by Friedrichs, and can be readily adapted by those skilled in the art for use with the therapeutic AF maintenance techniques described herein. Alternatively or additionally, AF is maintained using chemical/pharmacological agents known in the art, such as those described by Friedrichs in animal models, with appropriate modifications for treating AF in human patients.

In an embodiment of the present invention, AF is maintained using techniques described in the following articles, which are incorporated herein by reference: (a) Preston et al., entitled, “Permanent rapid atrial pacing to control supraventricular tachycardia,” Pacing Clin Electrophysiol, 2(3):331-334 (May 1979), and Moreira et al., entitled, “Chronic rapid atrial pacing to maintain atrial fibrillation: Use to permit control of ventricular rate in order to treat tachycardia induced cardiomyopathy,” Pacing Clin Electrophysiol, 12(5):761-775 (May 1989).

In an embodiment of the present invention, AF is maintained long-term, e.g., longer than about three weeks. Such AF maintenance generally reduces the frequency of recurring transitions between AF and NSR, which transitions are common in patients with AF, particularly in patients with chronic episodic AF. Such repeated transitions are generally undesirable because: (a) they often cause discomfort for the patient, (b) they may increase the risk of thromboembolic events, and (c) they often make prescribing an appropriate drug regimen difficult. Drug regimens that are beneficial for the patient when in AF are often inappropriate when the patient is in NSR, and vice versa. For example, beta blockers may help provide rate control for a patient when in AF, but may be harmful for the same patient when suffering bradycardia when in NSR. Knowledge that the patient will generally remain in AF typically helps a physician prescribe a more appropriate and/or lower-dosage drug regimen, in association with this embodiment. In addition, such AF maintenance may be beneficial for stabilizing a patient, such as a patient for whom cardioversion is not successful. For example, for many patients, electrical cardioversion alone is unsuccessful in maintaining NSR long-term (Fuster et al., Fuster V and Ryden L E et al., “ACC/AHA/ESC Practice Guidelines-—Executive Summary,” J Am Coll Cardiol 38(4):1231-65 (2001), and Fuster V and Ryden L E et al., “ACC/AHA/ESC Practice Guidelines—Full Text,” J Am Coll Cardiol 38(4):1266i-12661xx (2001), which are incorporated herein by reference, write that after undergoing cardioversion, “ . . . only 23% of the patients remained in sinus rhythm after 1 year and 16% after 2 years . . . ”).

In another embodiment of the present invention, AF is maintained short-term, typically between about one day and about three weeks. Such maintenance is generally beneficial during a period in which conventional anticoagulation drug therapy is applied to the patient prior to attempting electrical or pharmacological cardioversion. (Such a period may be desirable when an initial diagnosis of AF occurs more than 48 hours after initiation of AF, or an unknown amount of time after initiation of AF.) Cardioversion is generally not attempted during this period because of the particularly elevated risk of thromboembolic events before the anticoagulation therapy is effective. AF maintenance during this period to prevent naturally-occurring cardioversion, i.e., spontaneous reversion to NSR, is believed by the inventors to reduce the risk of thromboembolic events, such as stroke. Prior to attempting electrical or pharmacological cardioversion, the physician directs apparatus 20 to terminate AF maintenance.

In an embodiment of the present invention, control unit 32 drives electrode device 22 to apply signals to vagus nerve 24, and configures the signals so as to increase atrial motion. Such increased atrial motion typically causes mixing, such as by swirling or agitation of the blood in the atrium, which in turn is believed by the inventors to reduce the likelihood of coagulation and resultant thromboembolic events, including stroke (including in subjects having NSR). In an embodiment, control unit 32 modulates the vagal stimulation as follows:
during a “high” stimulation period, typically having a duration of between about 100 ms and about 1000 ms, the control unit configures the vagal stimulation so as to cause a reduction in the force of contraction of atrial cells; and
during a “low” stimulation period, typically having a duration of between about 200 ms and about 15 seconds, the control unit configures the vagal stimulation so as to cause the atrial cells to contract with “rebound” strength (although, because of the AF, the atrial cells typically remain unsynchronized during this rebound contraction).
   The resulting fluctuation in atrial contractility and pressure serves to mix the blood in the atria. For example, (a) the “high” period may have the following parameters: a duration of about 100 ms, a stimulation amplitude of about 5 milliamps, a pulse duration of about 1 ms, and a frequency of about 30 Hz; and (b) the “low” period may have the following parameters: a duration of about 12 seconds, and a stimulation amplitude of 0 milliamps (i.e., no stimulation during the “low” period). In this example, about 3 pulses are applied during a 100-ms period that occurs every 12 seconds.

In an embodiment, the control unit synchronizes the “high” and “low” periods with one or more sensed physiological variables, such as characteristics of the cardiac cycle or respiratory cycle. For example, the control unit may (a) initiate the “high” stimulation period within about 50 milliseconds after the occurrence of a QRS-complex, or within about 500 milliseconds after the beginning of an expiration, or (b) synchronize the “low” stimulation period with diastole, i.e., when the ventricle is open, in order to maximize blood flow from the atria.

Typically, the control unit configures the stimulation to cycle continuously between “high” and “low” stimulation when applying the treatment. The parameters of the modulation may include one or more of the following:
frequency—the stimulation is applied at a higher frequency during the “high” stimulation period than during the “low” stimulation period. For example, the “high” frequency may be about 20 Hz, while the “low” frequency may be about 1 Hz;
amplitude—the stimulation is applied with a higher amplitude during the “high” stimulation period than during the “low” stimulation period. For example, the “high” amplitude may be about 6 milliamps, while the “low” amplitude may be about 2 milliamps;
on/off—the stimulation is applied only during the “high” stimulation period;
induce/block—the stimulation is configured to induce action potentials in the vagus nerve during the “high” stimulation period, and to block action potentials in the vagus nerve during the “low” stimulation period;
pulse width—the stimulation is applied with a greater pulse width during the “high” stimulation period than during the “low” stimulation period. For example, the “high” pulse width may be about 1 ms, while the “low” pulse width may be about 0.2 ms; and/or
pulses per trigger (PPT)—the stimulation is applied at a higher PPT during the “high” stimulation period than during the “low” stimulation period. For example, the “high” PPT may be about 3 pulses per trigger, while the “low” PPT may be about 1 pulse per trigger.

Alternatively or additionally, control unit 32 increases atrial motion by electrical stimulation of cardiac tissue, such as atrial tissue or fat pads. Stimulation of left atrial tissue is typically achieved either by directly placing an electrode at or above the left auricle, or by stimulating the interatrial septum, the vena cava (e.g., in the area of the Ligament of Marshall), or the coronary sinus. Controllable parameters of such stimulation typically include frequency, amplitude, and/or on/off. For some applications, vagal and/or cardiac tissue stimulation is configured to improve blood flow out of the left atrial auricle. For example, electrical stimulation may be applied to the left atrial auricle for a short period during diastole at a frequency of at least about 3 Hz and at an amplitude greater than diastolic threshold.

For some applications, atrial motion is increased using the techniques described herein upon the termination of AF, for example, to prevent or treat electro-mechanical-dissociation (EMD), in which cardiac electrical activity is not coupled with appropriate mechanical contraction. Alternatively, atrial motion is increased using the techniques described herein in a patient who has not suffered from AF.

In an embodiment of the present invention, control unit 32 drives electrode device 22 to apply signals to vagus nerve 24, and configures the signals so as to restore NSR, i.e., to induce cardioversion. According to a first approach for restoring NSR, the configuration includes repeatedly changing parameters of the stimulation. The parameters changed may include one or more of the following:
intensity of stimulation (amplitude and/or frequency)—the strength of the stimulation is switched between stronger and weaker intensities;
on/off—the stimulation is configured to switch between applying stimulation and not applying stimulation, and/or a duration of an “on” period and/or an “off” period of the stimulation is varied;
pulse width of the stimulation; and/or
induce/block—the stimulation is configured to switch between inducing action potentials in the vagus nerve and blocking action potentials in the vagus nerve.

Typically, control unit 32 cycles between application of the different parameters at a rate of between about the duration of one heart beat and about 30 seconds. For some applications, the control unit performs the switching according to a predetermined pattern. For other applications, the control unit performs the switching randomly, with a typical interval between changes of between about 500 milliseconds and about 30 seconds.

Such switching of the stimulation is believed by the inventors to cause fluctuations in the atrial effective refractory period (AERP), thereby breaking reentry cycles and restoring synchronization and NSR. The inventors hypothesize that although the effect of vagal stimulation on the atria is generally heterogeneous in nature (not all areas of the atria receive the same stimulus), rapid switching of the stimulation, i.e., the application of heterogeneous stimuli, causes an overall atrial response that is more homogenous. The inventors further hypothesize that such atrial cell synchronization is due in part to: (a) more frequent activation of atrial cells because of the reduced refractory period caused by the vagal stimulation, and/or (b) the breaking of re-entry circuits during the brief periods when weak, blocking, or no vagal stimulation is applied.

According to a second approach for restoring NSR, control unit 32:
during a first period, typically having a duration between about 500 milliseconds and about 30 seconds, (a) paces the heart using conventional pacing techniques, such as by driving conventional pacemaker 42 to apply pacing signals to the heart, e.g., to the right atrium, right ventricle, or both ventricles, and, simultaneously, (b) configures the signals applied to the vagus nerve to provide generally constant vagal stimulation, i.e., without varying parameters of the stimulation, with a high intensity. Pacing of the heart is generally necessary because such high-intensity vagal stimulation would otherwise severely slow the heart rate; and
during a second period, suddenly ceases vagal stimulation. Such sudden cessation generally destabilizes the atrial cells, resulting in a return to NSR. The destabilization may be thought of as analogous to that achieved by conventional electrical cardioversion. The pacing is also generally terminated during the second period, typically simultaneously with, or up to about 30 seconds after, cessation of vagal stimulation. Alternatively, the pacing is terminated upon restoration of atrial activity.

The control unit may be configured to repeat this stimulation/pacing-sudden cessation cycle, if necessary to restore NSR.

A third approach is typically appropriate for treating AF principally caused by heightened adrenergic tone. When atrial fibrillation is induced by adrenergic tone, vagal stimulation generally reduces the net adrenergic effect by slowing the heart rate and by antagonizing the adrenergic system. According to this third approach, control unit 32 drives electrode device 22 to apply signals to vagus nerve 24, and configures the signals to apply substantially constant vagal stimulation, i.e., without varying parameters of the stimulation, so as to restore NSR. In this approach, the control unit typically does not use feedback in order to vary the parameters of stimulation. Parameters typically appropriate for such stimulation include: (a) application of a single pulse or a single burst of pulses each heart beat, (b) a pulse width of between about 0.5 ms and about 1.5 ms, and (c) a PPT of between about 1 and about 10. The amplitude of the applied signal is typically dependant upon the specific electrode device used for the treatment.

For all three of these approaches, the control unit may be configured to apply the cardioversion treatment: (a) upon detection of AF, (b) upon receiving an operator command, such as from a health care worker, or (c) at some other time. For some applications, the control unit applies the treatment at a certain time of day and/or when a patient motion signal received from accelerometer 39 indicates that the patient is at rest.

In an embodiment of the present invention, apparatus 20 is adapted to be used during conventional electrical atrial defibrillation. Control unit 32 drives electrode device 22 to apply stimulating signals to vagus nerve 24, and configures the stimulating signals to cause severe bradycardia and a decreased level of alertness during the defibrillation. Such severe bradycardia generally causes the patient to partially lose consciousness and thereby experience less pain during the defibrillation. Apparatus 20 thus can be thought of as a vagus nerve facilitated tranquilizer. Parameters for such stimulation are typically similar to those appropriate for heart rate reduction, however, with increased PPT. For example, such parameters may include a pulse width of between about 1 and about 3 milliseconds, such as about 2 milliseconds, an amplitude of between about 4 and about 8 milliamps, such as about 6 milliamps, and a PPT of between about 6 and about 10 pulses per trigger, such as about 8 pulses per trigger. Alternatively or additionally, parameters disclosed in the above-referenced U.S. patent application Ser. No. 10/205,475 are used. For some applications, apparatus 20 comprises conventional pacemaker 42, which is used to pace the heart in the event of excessive bradycardia caused by the vagal stimulation.

For some tranquilizing applications, control unit 32 additionally applies inhibiting signals to the vagus nerve, and configures the inhibiting signals to block vagal pain afferents, thereby further reducing pain experienced by the patient during the defibrillation. Techniques for selectively blocking pain sensations may be used that are described in (a) U.S. patent application Ser. No. 09/824,682, filed Apr. 4, 2001, now U.S. Pat. No. 6,600,954, (b) PCT Patent Application PCT/IL02/00068, filed Jan. 23, 2002, which published as PCT Publication WO 03/018113, and/or (c) U.S. patent application Ser. No. 09/944,913, filed Aug. 31, 2001, now U.S. Pat. No. 6,684,105, all of which are assigned to the assignee of the present patent application and are incorporated herein by reference.

FIG. 14 is a flow chart that schematically illustrates a method for determining and applying an appropriate AF treatment based on a countdown, in accordance with an embodiment of the present invention. In this embodiment, apparatus 20 additionally comprises a timer 43, which optionally is integrated in software of control unit 32 (FIG. 1). Alternatively, the functions of timer 43 may be implemented in circuitry of control unit 32. At an AF monitoring step 300, apparatus 20 monitors patient 30 for indications of AF, such as by using one or more of the AF detection techniques described hereinabove. So long as AF is not detected at an AF check step 302, the method returns to step 300. On the other hand, if AF is detected, control unit 32 records the time of initiation of the AF and optionally generates a notification signal, at a recording and notification step 304.

The control unit is typically adapted to report the recorded time of AF initiation and/or countdown time upon interrogation by a physician. If the patient seeks medical care after generation of the notification signal in step 304, the physician typically considers the recorded AF initiation time when determining the appropriate therapy. If the physician opts to attempt conventional cardioversion, the physician may reset the apparatus to resume monitoring for AF at step 300. Alternatively, the physician may opt to allow the device to continue its therapeutic course at step 306, as follows.

The control unit activates timer 43 to begin a countdown, at a countdown step 306. The countdown typically has a duration from the detection of AF of between about 24 and 54 hours, such as 48 hours. During the countdown, apparatus 20 typically attempts to restore NSR, using the cardioversion techniques and apparatus described herein, or other methods and apparatus known in the art, such as ICD 41. After attempting to restore NSR, at a success check step 310, the apparatus determines whether NSR has been successfully restored and maintained, such as by using one or more of the AF detection techniques described hereinabove. If NSR has been restored, the apparatus typically generates a notification signal to the patient and/or healthcare worker, at a notification generation step 312. The apparatus then resumes monitoring the patient for subsequent AF, at step 300.

On the other hand, if NSR has not been restored, then the apparatus checks whether the countdown has been completed, at a countdown check step 316. If the countdown has not been completed, the apparatus again attempts cardioversion, at step 308. For some applications, the apparatus is configured to pause between cardioversion attempts, and/or to make only a certain number of cardioversion attempts, typically based on programmed parameters and/or physiological parameters measured in real time. If, on the other hand, the countdown has concluded, the apparatus attempts to maintain AF, typically using AF maintenance techniques described herein, at an AF maintenance step 316. By minimizing or preventing undesired spontaneous transitions into NSR, the apparatus may reduce the risk of thromboembolic events, such as stroke. AF maintenance typically continues until a physician intervenes by signaling the apparatus to terminate maintenance, at an AF maintenance termination step 318.

For some applications, apparatus 20 is used with this countdown method in order to implement a set of clinical guidelines for treatment of AF. For example, the above-cited ACC/AHA/ESC practice guidelines for AF suggest that immediate cardioversion be attempted when AF has been present for less than 48 hours, but that the patient receive anticoagulation therapy for three to four weeks before cardioversion is attempted if the AF has been present for more than 48 hours. Such an anticoagulation period is also recommended when the duration of AF is unknown, for example, because the patient may have been asymptomatic for a period of time after initiation of AF. The use of this countdown method generally eliminates this unknown, thereby sometimes allowing beneficial cardioversion to be performed immediately rather than after three to four weeks of an anticoagulation drug regimen.

In an embodiment of the present invention, means are employed for avoiding bradycardia, which may be induced in response to application of some of the techniques described herein. Such means include, but are not limited to:
Applying stimulation only when the heart rate of the subject is greater than a minimum threshold, e.g., 60 beats per minute;
In the event that the heart rate drops below a threshold rate, e.g., 60 beats per minute, the heart is paced using conventional pacing techniques, such as by driving conventional pacemaker 42 to apply pacing signals to the heart, e.g., to the right atrium, right ventricle, or both ventricles, in order to keep the heart rate at or above the threshold value; and
Monitoring heart rate after applying stimulation. Upon detection that heart rate has fallen below a threshold rate, e.g., 60 beats per minute, during the following application of stimulation one or more parameters of the stimulation are adjusted so as to reduce the strength of the stimulation. For some applications, this technique is applied periodically or continuously while applying stimulation.

In an embodiment of the present invention (e.g., when the heart rate regulation algorithm described hereinabove is not implemented), to apply the closed-loop system, the target heart rate is expressed as a ventricular R-R interval (shown as the interval between R₁ and R₂ in FIG. 4). The actual R-R interval is measured in real time and compared with the target R-R interval. The difference between the two intervals is defined as a control error. Control unit 32 calculates the change in stimulation necessary to move the actual R-R towards the target R-R, and drives electrode device 22 to apply the new calculated stimulation. Intermittently, e.g., every 1, 10, or 100 beats, measured R-R intervals or average R-R intervals are evaluated, and stimulation of the vagus nerve is modified accordingly.

In an embodiment, apparatus 20 is further configured to apply stimulation responsive to pre-set time parameters, such as intermittently, constantly, or based on the time of day.

Alternatively or additionally, one or more of the techniques of smaller-to-larger diameter fiber recruitment, selective fiber population stimulation and blocking, and varying the intensity of vagus nerve stimulation by changing the stimulation amplitude, pulse width, PPT, and/or delay, are applied in conjunction with methods and apparatus described in one or more of the patents, patent applications, articles and books cited herein.

In an embodiment of the present invention, control unit 32 comprises or is coupled to an implanted device for monitoring and correcting the heart rate, such as an implantable cardioverter defibrillator (ICD) or a pacemaker (e.g., a bi-ventricular or standard pacemaker). For example, the implanted device may be incorporated into a control loop executed by control unit 32, in order to increase the heart rate when the heart rate for any reason is too low.

In an embodiment of the present invention, a method for increasing vagal tone comprises applying signals to vagus nerve 24, and configuring the signals to stimulate the vagus nerve, thereby delivering parasympathetic nerve stimulation to heart 28, while at the same time minimizing the heart-rate-lowering effects of the stimulation. Such treatment generally results in the beneficial effects of vagal stimulation that are not necessarily dependent on the heart-rate reduction effects of such stimulation. (See, for example, the above-cited article by Vanoli E et al.)

In an embodiment of the present invention, in order to increase vagal tone while at the same time minimizing or preventing the heart-rate-lowering effects of the stimulation, control unit 32 applies the signals to the vagus nerve as a burst of pulses during each cardiac cycle, with one or more of the following parameters:
Timing of the stimulation: delivery of the burst of pulses begins after a variable delay following each P-wave, the length of the delay equal to between about two-thirds and about 90% of the length of the patient's cardiac cycle. Such a delay is typically calculated on a real-time basis by continuously measuring the length of the patient's cardiac cycle.
Pulse duration: each pulse typically has a duration of between about 200 microseconds and about 2.5 milliseconds for some applications, or, for other applications, between about 2.5 milliseconds and about 5 milliseconds.
Pulse amplitude: the pulses are typically applied with an amplitude of between about 0.5 and about 5 milliamps, e.g., about 1 milliamp.
Pulse repetition interval: the pulses within the burst of pulses typically have a pulse repetition interval (the time from the initiation of a pulse to the initiation of the following pulse) of between about 2 and about 10 milliseconds, e.g., about 2.5 milliseconds.
Pulse period: the burst of pulses typically has a total duration of between about 0.2 and about 40 milliseconds, e.g., about 1 millisecond.
Pulses per trigger (PPT): the burst of pulses typically contains between about 1 and about 10 pulses, e.g., about 2 pulses.
Vagus nerve: the left vagus nerve is typically stimulated in order to minimize the heart-rate-lowering effects of vagal stimulation.
Duty cycle: stimulation is typically applied only once every several heartbeats (or once per heartbeat, when a stronger effect is desired).
On/off status: for some applications, stimulation is always “on”, i.e., constantly applied (in which case, parameters closer to the lower ends of the ranges above are typically used). For other applications, on/off cycles vary between a few seconds to several dozens of seconds, e.g., “on” for about 36 seconds, “off” for about 120 seconds, “on” for about 3 seconds, “off” for about 9 seconds.

For example, vagal stimulation may be applied to a patient having a heart rate of 60 BPM, with the intention of minimally reducing the patient's heart rate. The burst of pulses may be delivered beginning about 750 milliseconds after each R-wave of the patient. The stimulation may be applied with one pulse per trigger (PPT), and having an amplitude of 1 milliamp. The stimulation may be cycled between “on” and “off” periods, with each “on” period having a duration of about two seconds, i.e., two heart beats, and each “off” period having a duration of about 4 seconds.

Alternatively or additionally, the implanted device comprises a pacemaker, as described hereinabove with reference to FIG. 1, and control unit 32 drives the pacemaker to pace heart 28, so as to prevent any heart-rate lowering effects of such vagal stimulation. Typically, the control unit paces the heart at a rate that is similar to the rate when the device is in “off” mode. Control unit 32 then applies signals to vagus nerve 24, typically using the typical stimulation parameters described in the above-referenced U.S. patent application Ser. No. 10/866,601. This vagal stimulation generally does not lower the heart rate, because of the pacemaker pacing. For some applications, control unit 32 applies signals to vagus nerve 24, and senses the heart rate after applying the signals. The control unit drives the pacemaker to pace the heart if the sensed heart rate falls below a threshold heart rate. The threshold heart rate is typically equal to a heart rate of the patient prior to commencing the vagal stimulation, for example, as sensed by control unit 32. The control unit thus typically maintains the heart rate at a rate above a bradycardia threshold rate, unlike conventional pacemakers which are typically configured to pace the heart only when the rate falls below a bradycardia threshold rate. Upon termination of vagal stimulation, control unit 32 typically drives the pacemaker to continue pacing the heart for a period typically having a duration between about 0 and about 30 seconds, such as about 5 seconds.

In an embodiment of the present invention, apparatus 20 is adapted to be used prior to, during, and/or following a clinical procedure. Control unit 32 drives electrode device 22 to apply vagal stimulation, and typically configures the stimulation to reduce a potential immune-mediated response to the procedure. Such a reduction generally promotes healing after the procedure. (See Borovikova L V et al. cited hereinabove, which describe an anti-inflammatory cholinergic pathway that may mediate this reduction in immune-related response.) When the procedure is heart-related, the vagal stimulation additionally typically reduces mechanical stress by lowering heart rate and pressures, reduces heart rate, and/or improves coronary blood flow.

For some applications, the vagal stimulation commences after the conclusion of the procedure. For some applications, the vagal stimulation commences prior to the commencement of the procedure. Alternatively, the stimulation commences during the procedure. Further alternatively, the stimulation is applied before and after the procedure, but not during the procedure.

For some applications, the clinical procedure is selected from one of the following:
coronary artery bypass graft (CABG) surgery. In addition to the benefits of vagal stimulation described above, vagal tone was shown by Cumming J E et al. (cited hereinabove) to be effective in reducing the likelihood of postoperative atrial fibrillation (AF), increasing the likelihood that the graft will stay in place, reducing the likelihood of graft failure (e.g., via stenosis), improving healing from the surgery, and/or reducing pain associated with the surgery. It is hypothesized by the inventors that such a reduction in the likelihood of postoperative AF is due, at least in part, to the mechanical stress reduction and rhythmic vagal activity promoted by vagal stimulation. For some applications, the vagal stimulation is applied for between 1 and 7 days after the CABG surgery, intermittently or continuously.
valve replacement surgery. In addition to the benefits of vagal stimulation described above, vagal stimulation generally reduces the likelihood of postoperative AF, promotes healing of the heart, and reduces the likelihood of other conductance abnormalities.
heart transplantation. In addition to the benefits of vagal stimulation described above, vagal stimulation generally reduces the likelihood of rejection of the transplanted heart. For some applications, vagal stimulation is applied on a short-term basis, e.g., for less than about 7 days before and/or 7 days after the heart transplantation. Alternatively, vagal stimulation is applied long-term, e.g., for more than about 2 weeks before and/or 2 weeks after the procedure.
other organ transplantation, such as kidney, liver, skin grafting, and bone marrow transplantation. In addition to the benefits of vagal stimulation described above, vagal stimulation generally reduces the likelihood of rejection of the transplanted organ.
percutaneous transluminal coronary angioplasty (PTCA) and/or stenting procedures. In addition to the benefits of vagal stimulation described above, vagal stimulation generally reduces the likelihood of restenosis, which is believed to be at least in part immune-mediated. In addition, vagal stimulation induces coronary dilation, which generally reduces the likelihood of restenosis.
carotid endarterectomy. In addition to the benefits of vagal stimulation described above, vagal stimulation generally reduces the likelihood of restenosis, which is believed to be at least in part immune-mediated.
other bypass surgery. In addition to the benefits of vagal stimulation described above, vagal stimulation generally reduces the likelihood of restenosis in the grafted bypass (natural or artificial).
abdominal surgery. In addition to the benefits of vagal stimulation described above, vagal stimulation generally reduces the likelihood of narrowing of parts of the GI tract (a complication that often occurs after GI surgery, especially when anastomosis of GI components is performed).

In an embodiment of the present invention, control unit 32 drives electrode device 22 to apply vagal stimulation, and configures the stimulation to reduce hyperactivity or activity of brain cells, in order to treat conditions such as stroke and Attention Deficit Hyperactivity Disorder (ADHD). In one application, secondary stroke damage to cells in areas adjacent to the hypoxic area may be reduced by reducing the cell activity in these areas. In another application, vagal stimulation is configured to help reduce hyperactivity and improve concentration of a subject suffering from ADHD.

In an embodiment of the present invention, control unit 32 drives electrode device 22 to apply vagal stimulation, and configures the stimulation to treat one of the following conditions by reducing immune system hyperactivation associated with the condition:
vasculitis, e.g., Wegener granulomatosis, temporal arteritis, Takayasu arteritis, and/or polyarteritis nodosa;
systemic sclerosis;
systemic lupus erythematosus;
flare of Crohn's disease;
flare of ulcerative colitis;
autoimmune hepatitis;
glomerulonephritis;
arthritis, e.g., reactive or rheumatoid;
pancreatitis;
thyroiditis;
idiopathic thrombocytopenic purpura (ITP);
thrombotic thrombocytopenic purpura (TTP);
multi-organ failure associated with sepsis (especially gram negative sepsis);
anaphylactic shock;
Acute Respiratory Distress Syndrome (ARDS);
asthma;
an allergy—vagal stimulation is applied to attenuate allergic reactions of subjects suffering from acquired sensitizations to drugs or allergens, or from intense allergies. For some applications, apparatus 20 is configured to be an on-demand therapeutic adjuvant, e.g., to reduce the need for drug therapy; or
multiple sclerosis.

In an embodiment of the present invention, control unit 32 drives electrode device 22 to apply vagal stimulation, and configures the stimulation to treat a habitual behavior or a condition associated with a habitual behavior. The inventors hypothesize that vagal stimulation is effective for treating such behavior because the stimulation interferes with acquired habits or routines of the central nervous system (CNS). For some application, control unit 32 drives the electrode device to apply the stimulation at non-constant intervals, such as at random, quasi-random, or seemingly random intervals (e.g., generated using a random number generator or using a preselected set or pattern of varying intervals). The use of such variable intervals breaks cycles of the CNS responsible for such habitual behaviors. The use of non-constant intervals typically reduces the likelihood of the CNS cycle becoming synchronized with the stimulation, i.e., reduces the likelihood of accommodation.

Such habitual behaviors or behavior-related conditions include, but are not limited to:
anorexia, such as anorexia nervosa;
smoking;
drug addiction;
obsessive compulsive disorders;
intractable hiccups
sleep apnea;
Tourette syndrome; and
hiccups.

In an embodiment of the present invention, control unit 32 drives electrode device 22 to apply vagal stimulation that shifts the balance of the autonomic nervous system towards the parasympathetic side thereof, so as to modify the allocation of body resources among different organs and functions. Such vagal stimulation antagonizes the sympathetic system and augments the parasympathetic system, and may be applied in order to treat one or more of the following conditions:
hyperlipidemia—vagal stimulation is applied to promote lipid metabolism and absorption by the liver, and antagonizes the carbohydrate-based sympathetically-derived metabolism;
insulin resistance (e.g., type II diabetes)—the sympathetic system generally drives muscle tissue to increase its sensitivity to insulin. Vagal stimulation is applied to augment the parasympathetic system, thereby reducing the short-term sensitivity of muscle tissue to insulin. As a result, the long-term insulin sensitivity of muscle tissue increases;
chronic renal failure—vagal stimulation is applied to increase renal blood flow and glomerular filtration rate (GFR) by reducing blood flow to skeletal muscle (which blood flow is augmented by the sympathetic system), thereby allowing more blood to reach the kidneys, at lower pressures. For some applications, the vagal stimulation is applied while the patient sleeps, or is physically inactive, during which times the need for blood flow to skeletal muscle is reduced. Alternatively or additionally, vagal stimulation increases the GFR by acting on the kidney vascular bed;
chronic hepatic failure—vagal stimulation is applied to increase blood flow through the portal vein by reducing blood flow to skeletal muscle, thereby increasing blood flow through the liver. As a result, a compromised liver is able to perform additional work, and the condition of the patient improves. For some applications, the vagal stimulation is applied while the patient sleeps, or is physically inactive, during which times the need for blood flow to skeletal muscle is reduced;
insomnia—vagal stimulation is applied to shift the autonomic balance towards the parasympathetic system, allowing the mind and body to relax. Vagal stimulation promotes activities such as digestion, relaxation, and sleep;
muscle fatigue (such as associated with heart failure)—vagal stimulation is applied to reduce blood flow and energy consumption of skeletal muscles, thus allowing for muscle rest and recovery (similar to the manner in which beta blockers assist failing hearts);
muscle hypertonia—vagal stimulation is applied to reduce the tension in skeletal muscles, and/or to reduce the symptoms of hypertonia, such as hypertonia associated with upper motor neuron lesions;
sexual dysfunction—vagal stimulation is applied to increase the sensitivity of the sexual organs by increasing parasympathetic input, thereby promoting improved sexual function and/or pleasure;
anemia due to reduced production of red blood cells—vagal stimulation is applied to promote increased medullar red blood cell production and/or extramedullary red blood cell production. In unpublished data obtained from chronically vagal stimulated dogs, the inventors have shown increased extramedullary red blood cell production in response to chronic vagal stimulation; or
reduced peripheral blood flow—in contrast to the sympathetic system that augments blood flow to skeletal muscle, vagal stimulation reduces blood flow to skeletal muscle, thus augmenting the flow in peripheral blood vessels. In addition, parasympathetic stimulation has a direct effect of vasodilatation on peripheral blood vessels, further augmenting peripheral blood flow.

In an embodiment of the present invention, vagal stimulation is applied to treat stroke of a subject, such as by causing vasodilation. For some applications, such vagal stimulation is applied responsively to one or more sensed physiological parameters.

In an embodiment of the present invention, vagal stimulation is applied to treat a condition of a subject by regulating cell division of the subject. For some applications, the stimulation is configured to increase cell division to treat conditions including, but not limited to:
anemia;
a neurodegenerative disease;
liver cirrhosis;
an immune deficiency;
a skin burn or abrasion;
a muscle degenerative disorder;
cardiac failure; and
a reproductive system disorder.

For some applications, the stimulation is configured to decrease cell division to treat conditions including, but not limited to:
a neoplastic disorder;
a hematologic malignancy; and
polycythemia vera.

It has been suggested that cell cycle regulation is one of the humoral functions regulated by the vagus nerve. Preliminary data from animal experiments conducted by the inventors suggest that the vagus nerve regulates cell division. Such data include the incidence of splenomegaly in vagally-stimulated laboratory animals, and histological data from harvested cardiac tissue showing reduced levels of fibroblast growth among vagally-stimulated laboratory animals.

For some applications, when performing the vagal stimulation techniques described herein, vagal stimulation is applied for several hours, several days, several weeks, or longer. For some applications in which the vagal stimulation is applied on a short-term basis, a stimulating electrode is positioned in a manner that enables the expulsion of the electrode at the conclusion of the vagal stimulation treatment period. For some applications, the stimulating electrode is placed using a meltable or dissolvable suture or other element, which, when melted or dissolved at the completion of the treatment period, enables the electrode to be removed.

In an embodiment of the present invention, all or a portion of the electrode assembly, including conductive elements, is adapted to be dissolvable. When the dissolvable portion of the electrode assembly dissolves, the electrode assembly comes loose from the nervous tissue (e.g., the nerve), and the non-dissolvable portion of the electrode assembly, if any, can be removed. Appropriate dissolvable materials include polyglycolic acid (PGA) or poly(L-lactide) acid (PLL). For some applications, the portion of the electrode assembly that is within about 2 cm of the nervous tissue (e.g., the nerve) comprises entirely non-metal components, all or a portion of which are dissolvable. For some applications, the electrode assembly comprises electrode leads comprising metal wires, which are used to conduct the current through the body until a distance of about 2 cm from the nervous tissue (e.g., the nerve). For some applications, for conducting the current within about 2 cm of the nervous tissue (e.g., the nerve), the electrode assembly comprises electrode leads which comprise tubes (which are typically dissolvable) that contain an electrically conductive biologically-compatible liquid, such as saline solution. For some applications, in order to determine whether the dissolvable portion of the electrode assembly has dissolved sufficiently to enable safe removal of the remainder of the electrode assembly, the impedance of the assembly is measured.

In an embodiment of the present invention, apparatus 20 comprises an external stimulator, such as when a short period of activation is required. After completion of treatment, the external stimulator is disconnected from the subject, leaving only the electrodes implanted in the subject. For some applications, all or a portion of the electrodes dissolve, as described above, and/or all or a portion of the electrodes are removed from the subject. For some applications, apparatus 20 additionally comprises an external sensing element, such as an electrocardiogram (ECG) monitor, an electroencephalogram (EEG) monitor, a pulse oximeter, an ultrasound system, an MRI imaging system, a capnograph, a temperature sensor, a blood glucose monitor, a blood lipid monitor, a blood lactic acid monitor, or a blood urea, creatinine, or ammonia level monitor. For some applications, the external stimulator is adapted to be placed together with attached electrical leads in a sterile bag attached to the body at the site of insertion, which generally reduces the likelihood of infection.

In an embodiment of the present invention, apparatus 20 comprises an implantable stimulator comprising an internal battery. Alternatively or additionally, the implantable stimulator is powered with electromagnetically induced current, using an inducer external to the body. Further alternatively, apparatus 20 comprises one or more implantable electrodes that are activated by an external stimulator via magnetic induction.

In an embodiment of the present invention, apparatus 20 comprises a mechanical vibrator adapted to be placed external to the body, and to apply carotid massage in order to increase parasympathetic tone.

In an embodiment of the present invention, apparatus 20 comprises at least one electrode that is adapted to be positioned using vascular catheterization. For example, techniques described in one or more of the following articles may be used:
Vago H et al., “Parasympathetic cardiac nerve stimulation with implanted coronary sinus lead,” J Cardiovasc Elect 15:588-590 (2004)
Kara J et al., “Identification and characterization of atrioventricular parasympathetic innervation in humans,” Cardiovasc Elect 13:735-739 (2002)
Kara J. et al., “Characterization of sinoatrial parasympathetic innervation in humans,” J Cardiovasc Elect 10:1060-1065 (1999)

In an embodiment of the present invention, control unit 32 is configured to apply the vagal stimulation described hereinabove using one or more of the following techniques:
Control unit 32 configures the stimulation to be applied constantly, with a stimulation frequency between about 0.1 Hz and about 100 Hz, e.g., between about 0.1 Hz and about 5 Hz, or between about 5 Hz and about 100 Hz.
Control unit 32 synchronizes the stimulation with the cardiac cycle of subject 30, such as by using techniques described hereinabove and/or in one or more of the applications incorporated herein by reference.
Control unit 32 configures the stimulation using the minimal-heart-rate-lowering parameters described hereinabove.
Control unit 32 applies the stimulation only when the heart rate is above a threshold value, which is typically less than the average heart rate of subject 30, or less than the average heart rate of a typical subject.
Control unit 32 applies the stimulation intermittently, such as by using techniques described hereinabove and/or in one or more of the applications incorporated herein by reference.
Control unit 32 is configured to provide manual control of one or more of the stimulation parameters.

For some applications, techniques described herein are used to apply controlled stimulation to one or more of the following: the lacrimal nerve, the salivary nerve, the vagus nerve, the pelvic splanchnic nerve, or one or more sympathetic or parasympathetic autonomic nerves. Such controlled stimulation may be applied to such nerves directly, or indirectly, such as by stimulating an adjacent blood vessel or space. Such controlled stimulation may be used, for example, to regulate or treat a condition of the lung, heart, stomach, pancreas, small intestine, liver, spleen, kidney, bladder, rectum, large intestine, reproductive organs, or adrenal gland.

Although some embodiments of the present invention are described herein with respect to applying an electrical current to tissue of a subject, this is to be understood in the specification and in the claims as including creating a voltage drop between two or more electrodes.

In some embodiments of the present invention, techniques described herein for preventing and/or treating AF are used to prevent and/or treat atrial flutter, atrial premature beats (APBs), or other atrial arrhythmia.

Although embodiments of the present invention described hereinabove with reference to FIGS. 2A, 2C, 3 and 4 are described with reference to the vagus nerve, the electrode devices of these embodiments may also be applied to other nerves or nervous tissue for some applications, such as to the parasympathetic sites listed hereinabove.

The scope of the present invention includes embodiments described the references cited hereinabove in the Background of the Invention, and in the following applications, which are assigned to the assignee of the present application and are incorporated herein by reference. In an embodiment, techniques and apparatus described in one or more of the following applications are combined with techniques and apparatus described herein:
U.S. patent application Ser. No. 10/205,474, filed Jul. 24, 2002, entitled, “Electrode assembly for nerve control,” which published as US Patent Application Publication 2003/0050677
U.S. Provisional Patent Application 60/383,157 to Ayal et al., filed May 23, 2002, entitled, “Inverse recruitment for autonomic nerve systems”
U.S. patent application Ser. No. 10/205,475, filed Jul. 24, 2002, entitled, “Selective nerve fiber stimulation for treating heart conditions,” which published as US Patent Application Publication 2003/0045909
PCT Patent Application PCT/IL02/00068, filed Jan. 23, 2002, entitled, “Treatment of disorders by unidirectional nerve stimulation,” which published as PCT Publication WO 03/018113, and U.S. patent application Ser. No. 10/488,334, filed Feb. 27, 2004, in the US National Phase thereof, which issued as U.S. Pat. No. 7,734,355
U.S. patent application Ser. No. 09/944,913, filed Aug. 31, 2001, entitled, “Treatment of disorders by unidirectional nerve stimulation,” which issued as U.S. Pat. No. 6,684,105
U.S. patent application Ser. No. 10/461,696, filed Jun. 13, 2003, entitled, “Vagal stimulation for anti-embolic therapy,” which published as US Patent Application Publication 2004/0254612
PCT Patent Application PCT/IL03/00430, filed May 23, 2003, entitled, “Electrode assembly for nerve control,” which published as PCT Publication WO 03/099373
PCT Patent Application PCT/IL03/00431, filed May 23, 2003, entitled, “Selective nerve fiber stimulation for treating heart conditions,” which published as PCT Publication WO 03/099377
U.S. patent application Ser. No. 10/719,659, filed Nov. 20, 2003, entitled, “Selective nerve fiber stimulation for treating heart conditions,” which published as US Patent Application Publication 2004/0193231
PCT Patent Application PCT/IL04/00440, filed May 23, 2004, entitled, “Selective nerve fiber stimulation for treating heart conditions,” which published as PCT Publication WO 04/103455
PCT Patent Application PCT/IL04/000496, filed Jun. 10, 2004, entitled, “Vagal stimulation for anti-embolic therapy,” which published as PCT Publication WO 04/110550
U.S. patent application Ser. No. 10/866,601, filed Jun. 10, 2004, entitled, “Applications of vagal stimulation,” which published as US Patent Application Publication 2005/0065553
PCT Patent Application PCT/IL04/000495, filed Jun. 10, 2004, entitled, “Applications of vagal stimulation,” which published as PCT Publication WO 04/110549
U.S. patent application Ser. No. 11/022,011, filed Dec. 22, 2004, entitled, “Construction of electrode assembly for nerve control,” which published as US Patent Application Publication 2006/0136024
U.S. patent application Ser. No. 11/062,324, filed Feb. 18, 2005, entitled, “Techniques for applying, calibrating, and controlling nerve fiber stimulation,” which published as US Patent Application Publication 2005/0197675
U.S. patent application Ser. No. 11/064,446, filed Feb. 22, 2005, entitled, “Techniques for applying, configuring, and coordinating nerve fiber stimulation,” which published as US Patent Application Publication 2005/0267542
U.S. patent application Ser. No. 11/280,884, filed Nov. 15, 2005, entitled, “Techniques for nerve stimulation,” which published as US Patent Application Publication 2006/0106441
U.S. patent application Ser. No. 11/340,156, filed Jan. 25, 2006, entitled, “Method to enhance progenitor or genetically-modified cell therapy,” which published as US Patent Application Publication 2006/0167501
U.S. patent application Ser. No. 11/359,266, filed Feb. 21, 2006, entitled, “Parasympathetic pacing therapy during and following a medical procedure, clinical trauma or pathology,” which published as US Patent Application Publication 2006/0206155
U.S. patent application Ser. No. 10/745,514, filed Dec. 29, 2003, entitled, “Nerve-branch-specific action-potential activation, inhibition, and monitoring,” which published as US Patent Application Publication 2005/0149154
U.S. patent application Ser. No. 11/234,877, filed Sep. 22, 2005, entitled, “Selective nerve fiber stimulation,” which published as US Patent Application Publication 2006/0100668

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method comprising:
identifying that a subject is at risk of suffering from atrial fibrillation (AF); and
responsively to the identifying, reducing a risk of an occurrence of an episode of the AF by:
coupling an electrode device to a site of a subject containing parasympathetic nervous tissue,
driving, by a control unit, the electrode device to apply an electrical current to the site not responsively to any physiological parameters sensed by any device directly or indirectly coupled to the control unit, and
configuring the current to stimulate autonomic nervous tissue in the site.

2. The method according to claim 1, wherein the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and wherein applying the current comprises applying the current to the selected site.

3. The method according to claim 1, wherein the site includes the vagus nerve, and wherein applying the current comprises applying the current to the vagus nerve.

4. Apparatus comprising:
an electrode device, configured to be coupled to a site of the subject at risk of suffering from atrial fibrillation (AF), the site containing parasympathetic nervous tissue; and
a control unit, configured to reduce a risk of an occurrence of an episode of the AF by:
driving the electrode device to apply an electrical current to the site not responsively to any physiological parameters sensed by any device directly or indirectly coupled to the control unit, and
configuring the current to stimulate the nervous tissue in the site.

5. The apparatus according to claim 4, wherein the site is selected from the group consisting of: a vagus nerve, an epicardial fat pad, a sinoatrial (SA) node fat pad, a pulmonary vein, a carotid artery, a carotid sinus, a coronary sinus, a vena cava vein, a jugular vein, an azygos vein, an innominate vein, and a subclavian vein, and wherein the electrode device is configured to be coupled to the selected site.

6. The apparatus according to claim 4, wherein the site includes the vagus nerve, and wherein the electrode device is configured to be coupled to the vagus nerve.

7. A method for treating a subject, comprising:
applying a current to a site of the subject in respective bursts of pulses in each of a plurality of cardiac cycles of the subject, the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and
configuring an electrical parameter of the current so as to minimize an effect of the applying of the current on a heart rate of the subject, by applying each of the bursts after a delay following a P-wave of the subject, the delay having a duration equal to between about two-thirds and about 90% of a duration of a cardiac cycle of the subject.

8. The method according to claim 7, wherein applying the current to the site of the subject comprises applying the current to the site of a subject who suffers from a condition selected from the list consisting of: an autoimmune disease, an autoimmune inflammatory disease, multiple sclerosis, encephalitis, myelitis, immune-mediated neuropathy, myositis, dermatomyositis, polymyositis, inclusion body myositis, inflammatory demyelinating polyradiculoneuropathy, Guillain Barre syndrome, myasthenia gravis, inflammation of the nervous system, inflammatory bowel disease, Crohn's disease, ulcerative colitis, SLE (systemic lupus erythematosus), rheumatoid arthritis, vasculitis, polyarteritis nodosa, Sjogren syndrome, mixed connective tissue disease, glomerulonephritis, thyroid autoimmune disease, sepsis, meningitis, a bacterial infection, a viral infection, a fungal infection, sarcoidosis, hepatitis, and portal vein hypertension.

9. The method according to claim 7, wherein applying the current comprises configuring the pulses within each of the bursts to have a pulse repetition interval of between 2 and 10 milliseconds.

10. The method according to claim 7, wherein applying the current comprises applying the bursts less than every heartbeat of the subject.

11. Apparatus for treating a subject, comprising:
an electrode device, adapted to be coupled to a site of the subject selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject; and
a control unit, adapted to:
drive the electrode device to apply an electrical current to the site in respective bursts of pulses in each of a plurality of cardiac cycles of the subject, and configure an electrical parameter of the current so as to minimize an effect of the applying of the current on a heart rate of the subject, by applying each of the bursts after a variable delay following a P-wave of the subject, the delay having a duration equal to between about two-thirds and about 90% of a duration of a cardiac cycle of the subject.

12. The apparatus according to claim 11, wherein the control unit is adapted to configure the pulses within each of the bursts to have a pulse repetition interval of between 2 and 10 milliseconds.

13. The apparatus according to claim 11, wherein the control unit is adapted to apply the bursts less than every heartbeat of the subject.

14. Treatment apparatus, comprising:
an electrode device, adapted to be coupled to a site of a subject suffering from atrial fibrillation (AF), the site selected from the list consisting of: a vagus nerve of the subject, an epicardial fat pad of the subject, a pulmonary vein of the subject, a carotid artery of the subject, a carotid sinus of the subject, a vena cava vein of the subject, and an internal jugular vein of the subject;
a pacing device, adapted to be applied to a heart of the subject; and
a control unit, adapted to:
during a first period, drive the pacing device to pace the heart, and drive the electrode device to apply an electrical current to the site, and
during a second period following the first period, withhold the electrode device from applying the electrical current to the site.

15. Apparatus according to claim 14, wherein the control unit is adapted to configure a parameter of at least one of the periods to be such as to restore normal sinus rhythm (NSR) of the subject within 2 hours after initiation of the second period.

16. Apparatus according to claim 15, wherein the site is a vagus nerve, and wherein the electrode device is adapted to be applied to the vagus nerve.

17. Apparatus according to claim 14, wherein the control unit is adapted to withhold the pacing device from pacing the heart during at least a portion of the second period.

18. Apparatus according to claim 14, wherein the control unit is adapted to configure the first period to have a duration of between about 500 milliseconds and about 30 seconds.

19. Apparatus according to claim 14, wherein the control unit is adapted to drive the electrode device to apply the electrical current substantially without changing the parameter during the first period, and with an amplitude greater than about 6 milliamps.

20. Apparatus according to claim 14, further comprising a sensor, adapted to detect an occurrence of the AF and generate a sensor signal indicative thereof, and wherein the control unit is adapted to receive the sensor signal, and to drive the pacing device and drive the electrode device to apply the electrical current responsive to the sensor signal.

21. Apparatus according to claim 14, further comprising a sensor, adapted to detect an occurrence of the AF and generate a sensor signal indicative thereof, and wherein the control unit is adapted to receive the sensor signal, and to withhold the electrode device from applying the electrical current responsive to the sensor signal.

22. Apparatus comprising an electrode assembly adapted to be coupled to nervous tissue of a subject, the electrode assembly comprising one or more conductive elements, wherein at least a portion of the electrode assembly is adapted to be dissolvable after the electrode assembly has been coupled to the tissue.

23. The apparatus according to claim 22, wherein the nervous tissue includes a nerve of the subject, and wherein the electrode assembly is adapted to be coupled to the nerve.

24. The apparatus according to claim 22, wherein the electrode assembly is adapted to come loose from the tissue upon dissolving of the dissolvable at least a portion thereof.

25. A method comprising:
providing an electrode assembly including one or more conductive elements, at least a portion of which electrode assembly is configured to be dissolvable after the electrode assembly has been coupled to nervous tissue of a subject; and
coupling the electrode assembly to the nervous tissue.

26. Apparatus for treating a subject, comprising:
an electrode device, configured to be coupled to a parasympathetic site of the subject selected from the group consisting: of a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject; and
a control unit, configured to:
drive the electrode device to apply a current to the site,
receive a sensed physiological value of the subject selected from the group consisting of: a temperature of the subject, a blood glucose level of the subject, a blood lipid level of the subject, a blood lactic acid level of the subject, a blood CO2 level of the subject, a blood O2 level of the subject, a blood urea level of the subject, a blood creatinine level of the subject, and a blood ammonia level of the subject, and set at least one parameter of the applied current responsively to the sensed physiological value.

27. A method for treating a subject, comprising:
applying a current to a parasympathetic site of the subject selected from the group consisting: of a vagus nerve of the subject, an epicardial fat pad of the subject, a pulmonary vein of the subject, a carotid artery of the subject, a carotid sinus of the subject, a coronary sinus of the subject, a vena cava vein of the subject, a jugular vein of the subject, a right ventricle of the subject, a parasympathetic ganglion of the subject, and a parasympathetic nerve of the subject;
receiving a sensed physiological value of the subject selected from the group consisting of: a temperature of the subject, a blood glucose level of the subject, a blood lipid level of the subject, a blood lactic acid level of the subject, a blood CO2 level of the subject, a blood O2 level of the subject, a blood urea level of the subject, a blood creatinine level of the subject, and a blood ammonia level of the subject; and
setting at least one parameter of the applied current responsively to the sensed physiological value.
